(19) United States
(12) Patent Application Publication
(10) Pub. No.: US 2025/0034568 A1
(43) Pub. Date: Jan. 30, 2025

(54) CONSTRUCTS COMPRISING TANDEM MICRORNA-ADAPTED SHORT HAIRPIN RNA (SHMIR) FOR INCREASING FETAL HEMOGLOBIN

(71) Applicants: THE CHILDREN'S MEDICAL CENTER CORPORATION, Boston, MA (US); DANA-FARBER CANCER INSTITUTE, Boston, MA (US)

(72) Inventors: David A. WILLIAMS, Dover, MA (US); Daniel E. BAUER, Cambridge, MA (US); Christian BRENDEL, Boston, MA (US)

(73) Assignees: THE CHILDREN'S MEDICAL CENTER CORPORATION, Boston, MA (US); DANA-FARBER CANCER INSTITUTE, Boston, MA (US)

(21) Appl. No.: 18/707,354

(22) PCT Filed: Oct. 14, 2022
(86) PCT No.: PCT/US2022/046743

§ 371 Date: May 3, 2024

Related U.S. Application Data
(60) Provisional application No. 63/275,095, filed on Nov. 3, 2021.
(60) Provisional application No. 63/318,931, filed on Mar. 11, 2022.

(51) Int. Cl. C12N 15/113; A61P 7/00; A61P 7/06; C12N 15/86

(52) U.S. Cl. CPC C12N 15/113; A61P 7/00; A61P 7/06; C12N 15/86; C12N 2310/141; C12N 2310/531; C12N 2740/15043

(57) ABSTRACT

The technologies as described herein relate to compositions and methods for the treatment of hemoglobinopathies by increasing fetal hemoglobin in a subject.

### CROSS-REFERENCE TO RELATED APPLICATIONS

This application is a 35 U.S.C. § 371 National Phase Entry Application of International Application No. PCT/US2022/046743 filed Oct. 14, 2022, which designates the U.S., and which claims benefit under 35 U.S.C. § 119(e) of the U.S. Provisional Application No. 63/275,095, filed Nov. 3, 2021 and U.S. Provisional Application No. 63/318,931, filed Mar. 11, 2022, the contents of which are incorporated herein by reference in their entireties.

### SEQUENCE LISTING

The instant application contains a Sequence Listing which has been submitted electronically in XML format and is hereby incorporated by reference in its entirety. Said XML copy, created on Dec. 6, 2022, is named 701039-099670_SL.xml and is 73,728 bytes in size

### TECHNICAL FIELD

Embodiments disclosed herein relate to compositions and methods for the treatment of hemoglobinopathies by increasing fetal hemoglobin in a subject.

### BACKGROUND

Hemoglobinopathies, including sickle cell disease/anemia (SCD) and thalassemia (THAL), are the most prevalent inherited monogenic disorders in humans. Approximately 5% of the world's population carries a globin gene mutation. The World Health Organization estimates that each year about 300,000 infants are born with major hemoglobin disorders. Due to historic and/or recent migration, increasing numbers of patient populations can now be found in developed countries, and public health implications of SCD are significant (Kauf et al., American Journal of Hematology. 2009; 84:323-327; Amendah et al., American Journal of Preventive Medicine. 2010; 38:S550-556). In the United States, the median survival of patients having a hemoglobinopathy was estimated in 1994 to be 42 years for men and 48 years for women (Platt et al., New England Journal of Medicine. 1994; 330:1639-1644).

At a molecular level, SCD was the first disease to be linked to a molecular alteration (Pauling et al., Science. 1949; 110:543-548). A single nucleotide mutation results in a glutamic acid to valine substitution at position 6 of the β-globin protein. This modification results in the polymerization of the molecule in deoxygenated conditions, and subsequent “sickling” of the erythrocyte ultimately leading to anemia by hemolysis and acute and chronic vaso-occlusive and ischemic complications affecting multiple organs, including kidney, brain, lung, and others). Although preventive measures (including the chemoprophylactic agent hydroxyurea) have led to moderate reduction in the burden of selected patient groups, at present, the only available curative therapy for SCD is allogeneic hematopoietic stem cell transplantation (HSCT) (Hsieh et al., New England Journal of Medicine. 2009; 361:2309-2317; Hsieh et al., Blood; Electronic pre-publication Jun. 31, 2011). HSCT has unfortunately been associated in the SCD and THAL setting with significant mortality and morbidity, which is due in part to pre-HSCT transfusion-related iron overload, graft-versus-host disease, and high doses of chemotherapy/radiation required for pre-transplant conditioning of the host, among others. Thus, it is important to develop alternative therapeutics to treat sickle cell anemia.

BCL11A promotes the transitional switch from the expression of fetal hemoglobin genes to the expression of adult hemoglobin genes during fetal development. Suppression of BCL11A reverses this switch and induces a significantly higher expression of the fetal hemoglobin genes post fetal development. The higher amount of fetal hemoglobin expressed ameliorates the sickling phenotype of red cells and thus the symptoms associated with various β-hemoglobinopathies.

### SUMMARY

The methods and compositions provided herein are based, in part, on the discovery that administering a construct comprising at least two tandem microRNA-adapted short hairpin RNAs (also referred to herein as a “double-shmiR”) targeting BCL11A and ZNF-410 was more effective in the treatment of a hemoglobinopathy than treatment with a single microRNA-adapted short hairpin RNA (shmiR) targeting BCL11A alone.

Provided herein, in one aspect is a nucleic acid vector encoding a first and at least a second microRNA-adapted short hairpin RNA (shmiR) in tandem.

In one embodiment of this aspect and all other aspects provided herein, the first shmiR comprises a first short hairpin mRNA (shRNA) embedded in a framework of a first miRNA sequence, and the at least second shmiR comprises a second shRNA embedded in a framework of a second miRNA sequence.

In another embodiment of this aspect and all other aspects provided herein, the first shmiR comprises a first segment from the first miRNA, a first BCL11a segment, a loop segment of the first miRNA sequence, a second BCL11a segment, and a second segment from the first miRNA arranged in tandem in a 5′ to 3′ direction, wherein the loop segment is between and directly linked to the first and second BCL11a segments, and wherein the sequence of the second BCL11A segment is complementary to the sequence of the first BCL11a segment, and wherein the sequence of the first segment of the first miRNA is complementary to the sequence of the second segment of the first miRNA, and wherein (i) the first and second segments of the first miRNA and (ii) the first and second BCL11a segments base pair to form a hairpin loop, with the loop segment of the first miRNA forming the loop portion of the hairpin loop thus formed.

In another embodiment of this aspect and all other aspects provided herein, the second shmiR comprises a first segment from the at least second miRNA, a first ZNF410 or ZBTB7A segment, a loop segment of the at least second miRNA, a second, complementary ZNF410 or ZBTB7A segment, and a second segment of the at least second miRNA arranged in tandem in a 5′ to 3′ direction, wherein the loop segment is between and directly linked to the first and second ZNF410 or ZBTB7A segments, and wherein the sequence of the first segment of the at least second miRNA is complementary to the sequence of the second segment of the at least second miRNA, wherein the sequence of the first segment of ZNF410 or ZBTB7a is complementary to the second segment of ZNF410 or ZBTB7a and, wherein (i) the first and third segments of the at least second miRNA and (ii) the first and second ZNF410 or ZBTB7 segments base pair to form a hairpin loop, with the loop segment of the at least second miRNA forming the loop portion of the hairpin loop thus formed.

In another embodiment of this aspect and all other aspects provided herein, the first miRNA sequence comprises miR223.

In another embodiment of this aspect and all other aspects provided herein, the at least second miRNA sequence comprises miR144.

In another embodiment of this aspect and all other aspects provided herein, the first miRNA sequence comprises miR223 and the second miRNA sequence comprises miR144.

In another embodiment of this aspect and all other aspects provided herein, the first shRNA comprises a BCL11a targeting sequence.

In another embodiment of this aspect and all other aspects provided herein, the second shRNA comprises a ZNF410 targeting sequence or a ZBTB7A targeting sequence.

In another embodiment of this aspect and all other aspects provided herein, the first and second BCL11a, ZNF410 or ZBTB7A segments are each 18-25 nucleotides long.

In another embodiment of this aspect and all other aspects provided herein, the first segment of the miR223 miRNA comprises the sequence of:

(SEQ ID NO: 1)
GATCTCACTTCCCCACAGAAGCTCTTGGCCTGGCCTCCTGCAGT
GCCACGCT.

In another embodiment of this aspect and all other aspects provided herein, the loop segment comprises a segment of miR223. In one embodiment of this aspect and all other aspects provided herein, the miR223 loop segment comprises the sequence of:

(SEQ ID NO: 2)
CTCCATGTGGTAGAG.

In another embodiment of this aspect and all other aspects provided herein, the second segment of the miR223 miRNA comprises the sequence of:

(SEQ ID NO: 3)
AGTGCGGCACATGCTTACCAGCTCTAGGCCAGGGCAGATGGGAT
ATGACGAATGGACTGCCAGCTGGATACAAGGATGCTCACC.

In another embodiment of this aspect and all other aspects provided herein, the first BCL11A sequence comprises the sequence of: GCGCGATCGAGTGTTGAATAA (SEQ ID No: 4) and the second BCL11A sequence comprises the sequence of: TTATTCAACACTCGATCGCGC (SEQ ID NO: 5), wherein the first and second BCL11A sequence are complementary.

In another embodiment of this aspect and all other aspects provided herein, the BCL11 A sequence in an miR223 framework comprises the sequence of:

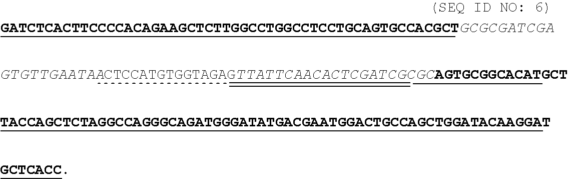

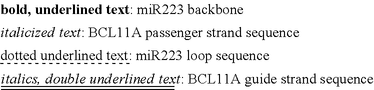

In another embodiment of this aspect and all other aspects provided herein, the first segment of the miR144 miRNA comprises the sequence of:

(SEQ ID NO: 7)
CGCTTTTCAAGCCATGCTTCCTGTGCCCCCAGTGGGGCCCTGGCTG.

In another embodiment of this aspect and all other aspects provided herein, the loop segment comprises a segment of miR144. In one embodiment of this aspect and all other aspects provided herein, the miR144 loop segment comprises the sequence of AGTTTGCGATGAGACAC (SEQ ID NO: 8).

In another embodiment of this aspect and all other aspects provided herein, the second segment of the miR144 miRNA comprises the sequence of:

(SEQ ID NO: 9)
AGTCCGGGCACCCCCAGCTCTGGAGCCTGACAAGGAGGACAGGAGAGAT.

In another embodiment of this aspect and all other aspects provided herein, the first ZNF410 sequence comprises the sequence of: GCTGAGCACTTAGTGTTTGTA (SEQ ID No: 10) and the second ZNF410 sequence comprises the sequence of: TACAAACACTAAGTGCTCAGC (SEQ ID NO: 11), wherein the first and second ZNF410 sequence are complementary.

In another embodiment of this aspect and all other aspects provided herein, the ZNF410 sequence in an miR144 framework comprises the sequence of:

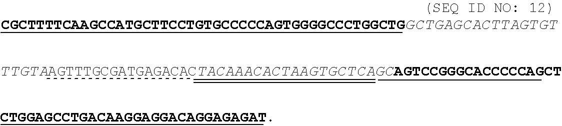

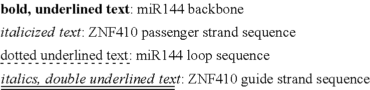

In another embodiment of this aspect and all other aspects provided herein, the first ZBTB7A sequence comprises the sequence of: ACGGGTACTTTTCATTCGCGC (SEQ ID No: 13) and the second ZBTB7A sequence comprises the sequence of: GCGCGAATGAAAAGTACCCGT (SEQ ID NO: 14), wherein the first and second ZBTB7A sequence are complementary.

In another embodiment of this aspect and all other aspects provided herein, the ZBTB7A sequence in an miR144 framework comprises the sequence of:

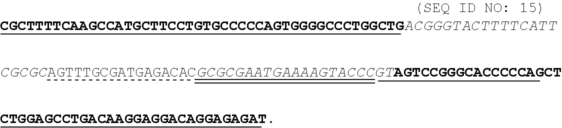

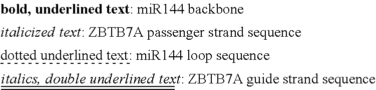

In another embodiment of this aspect and all other aspects provided herein, the shmiR that targets BCL11A comprises the sequence of SEQ ID NO: 16.

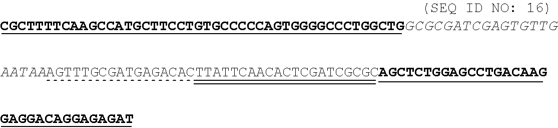

Within the sequence of SEQ ID NO: 16, the bold, underlined text is an miRNA 144 backbone sequence, the italics text is a passenger strand sequence, the [symbol] text is a miRNA 144 loop sequence (SEQ ID NO: 8), and the double underlined text is a guide strand sequence.

In another embodiment of this aspect and all other aspects provided herein, the first and second BCL11a, ZNF410 or ZBTB7A segments are complementary.

In another embodiment of this aspect and all other aspects provided herein, the first shmiR and the second shmiR do not undergo homologous recombination when introduced into a cell.

Another aspect provided herein relates to an RNA transcript expressed from the nucleic acid vector as described herein.

Also provided herein, in another aspect, is an RNA transcript comprising a first and at least a second microRNA-adapted short hairpin RNA (shmiR).

In one embodiment of this aspect and all other aspects provided herein, the first shmiR comprises a first short hairpin mRNA (shRNA) embedded in a framework of a first miRNA sequence, and the at least second shmiR comprises a second shRNA embedded in a framework of a second miRNA sequence.

In another embodiment of this aspect and all other aspects provided herein, the first shmiR comprises a first segment from the first miRNA, a first BCL11a segment, a loop segment of the first miRNA sequence, a second BCL11a segment, and a second segment from the first miRNA arranged in tandem in a 5′ to 3′ direction, wherein the loop segment is between and directly linked to the first and second BCL11a segments, and wherein the sequence of the second BCL11A segment is complementary to the sequence of the first BCL11a segment, and wherein the sequence of the first segment of the first miRNA is complementary to the sequence of the second segment of the first miRNA, and wherein (i) the first and second segments of the first miRNA and (ii) the first and second BCL11a segments base pair to form a hairpin loop, with the loop segment of the first miRNA forming the loop portion of the hairpin loop thus formed.

In another embodiment of this aspect and all other aspects provided herein, the second shmiR comprises a first segment from the at least second miRNA, a first ZNF410 or ZBTB7A segment, a loop segment of the at least second miRNA, a second, complementary ZNF410 or ZBTB7A segment, and a second segment of the at least second miRNA arranged in tandem in a 5′ to 3′ direction, wherein the loop segment is between and directly linked to the first and second ZNF410 or ZBTB7A segments, and wherein the sequence of the first segment of the at least second miRNA is complementary to the sequence of the second segment of the at least second miRNA, wherein the sequence of the first segment of ZNF410 or ZBTB7a is complementary to the second segment of ZNF410 or ZBTB7a and, wherein (i) the first and third segments of the at least second miRNA and (ii) the first and second ZNF410 or ZBTB7 segments base pair to form a hairpin loop, with the loop segment of the at least second miRNA forming the loop portion of the hairpin loop thus formed.

In another embodiment of this aspect and all other aspects provided herein, the first miRNA sequence comprises miR223.

In another embodiment of this aspect and all other aspects provided herein, the at least second miRNA sequence comprises miR144.

In another embodiment of this aspect and all other aspects provided herein, the first miRNA sequence comprises miR223 and the second miRNA sequence comprises miR144.

In another embodiment of this aspect and all other aspects provided herein, the first shRNA comprises a BCL11a targeting sequence.

In another embodiment of this aspect and all other aspects provided herein, the second shRNA comprises a ZNF410 targeting sequence or a ZBTB7A targeting sequence.

In another embodiment of this aspect and all other aspects provided herein, the first and second BCL11a, ZNF410 or ZBTB7A segments are each 18-25 nucleotides long.

In another embodiment of this aspect and all other aspects provided herein, the first segment of the miR223 miRNA comprises the sequence of

(SEQ ID NO: 1)
GATCTCACTTCCCCACAGAAGCTCTTGGCCTGGCCTCCTGCAGTGCCA
CGCT.

In another embodiment of this aspect and all other aspects provided herein, the loop segment comprises a segment of miR223. In one embodiment of this aspect and all other aspects provided herein, the miR223 loop segment comprises the sequence of:

(SEQ ID NO: 2)
CTCCATGTGGTAGAG.

In another embodiment of this aspect and all other aspects provided herein, the second segment of the miR223 miRNA comprises the sequence of:

(SEQ ID NO: 3)
AGTGCGGCACATGCTTACCAGCTCTAGGCCAGGGCAGATGGGATA
TGACGAATGGACTGCCAGCTGGATACAAGGATGCTCACC.

In another embodiment of this aspect and all other aspects provided herein, the first BCL11A sequence comprises the sequence of: GCGCGATCGAGTGTTGAATAA (SEQ ID No: 4) and the second BCL11A sequence comprises the sequence of: TTATTCAACACTCGATCGCGC (SEQ ID NO: 5), wherein the first and second BCL11A sequence are complementary.

In another embodiment of this aspect and all other aspects provided herein, the BCL11 A sequence in an miR223 framework comprises the sequence of:

(SEQ ID NO: 6)
GATCTCACTTCCCCACAGAAGCTCTTGGCCTGGCCTCCTGCAGTGCCACGCTGCGCGATCGA

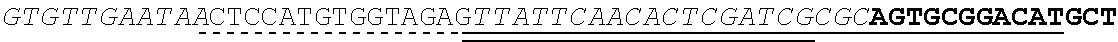

TACCAGCTCTAGGCCAGGGCAGATGGGATATGACGAATGGACTGCCAGCTGGATACAAGGAT
GCTCACC.
bold, underlined text: miR223 backbone
italicized text: BCL11A passenger strand sequence

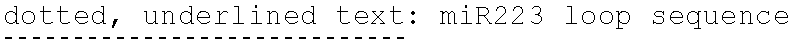

italics, double underlined text: BCL11A guide strand sequence

In another embodiment of this aspect and all other aspects provided herein, the first segment of the miR144 miRNA comprises the sequence of:

(SEQ ID NO: 7)
CGCTTTTCAAGCCATGCTTCCTGTGCCCCCAGTGGGGCCCTGGCTG.

In another embodiment of this aspect and all other aspects provided herein, the loop segment comprises a segment of miR144. In one embodiment of this aspect and all other aspects provided herein, the miR144 loop segment comprises the sequence of AGTTTGCGATGAGACAC (SEQ ID NO: 8).

In another embodiment of this aspect and all other aspects provided herein, the second segment of the miR144 miRNA comprises the sequence of:

(SEQ ID NO: 9)
AGTCCGGGCACCCCCAGCTCTGGAGCCTGACAAGGAGGACAGGAGAGAT.

In another embodiment of this aspect and all other aspects provided herein, the first ZNF410 sequence comprises the sequence of: GCTGAGCACTTAGTGTTTGTA (SEQ ID No: 10) and the second ZNF410 sequence comprises the sequence of: TACAAACACTAAGTGCTCAGC (SEQ ID NO: 11), wherein the first and second ZNF410 sequence are complementary.

In another embodiment of this aspect and all other aspects provided herein, the ZNF410 sequence in an miR144 framework comprises the sequence of:

(SEQ ID NO: 12)
CGCTTTTCAAGCCATGCTTCCTGTGCCCCCAGTGGGGCCCTGGCTGGCTGAGCACTTAGTGT

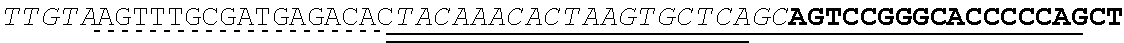

CTGGAGCCTGACAAGGAGGACAGGAGAGAT.
bold, underlined text: miR144 backbone
italicized text: ZNF410 passenger strand sequence

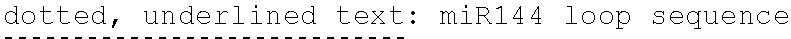

italics, double underlined text: ZNF410 guide strand sequence

In another embodiment of this aspect and all other aspects provided herein, the first ZBTB7A sequence comprises the sequence of: ACGGGTACTTTTCATTCGCGC (SEQ ID No: 13) and the second ZBTB7A sequence comprises the sequence of: GCGCGAATGAAAAGTACCCGT (SEQ ID NO: 14), wherein the first and second ZBTB7A sequence are complementary.

In another embodiment of this aspect and all other aspects provided herein, the ZBTB7A sequence in an miR144 framework comprises the sequence of:

(SEQ ID NO: 15)
CGCTTTTCAAGCCATGCTTCCTGTGCCCCCAGTGGGGCCCTGGCTGACGGGTACTTTTCATT

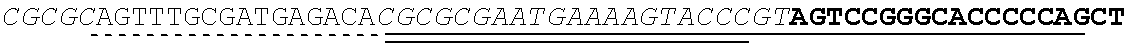

CTGGAGCCTGACAAGGAGGACAGGAGAGAT.
bold, underlined text: miR144 backbone
italicized text: ZBTB7A passenger strand sequence

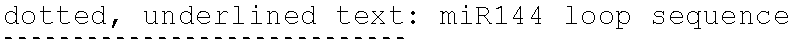

italics, double underlined text: ZBTB7A guide strand sequence

In another embodiment of this aspect and all other aspects provided herein, the shmiR that targets BCL11A comprises the sequence of SEQ ID NO: 16.

(SEQ ID NO: 16)
CGCTTTTCAAGCCATGCTTCCTGTGCCCCCAGTGGGGCCCTGGC

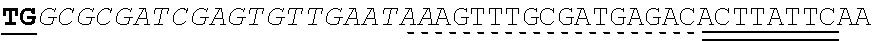

CACTCGATCGCGCAGTCCGGGCACCCCCAGCTCTGGAGCCTGACA
AGGAGGACAGGAGAGAT

Within the sequence of SEQ ID NO: 16, the bold, underlined text is an miRNA 144 backbone sequence, the italics text is a passenger strand sequence, the [symbol] text is a miRNA 144 loop sequence (SEQ ID NO: 8), and the double underlined text is a guide strand sequence.

Another aspect provided herein relates to a method for treating or reducing at least one symptom of a hemoglobinopathy, the method comprising: administering a nucleic acid vector or an RNA transcript as described herein to a subject in need thereof, thereby treating or reducing at least one symptom of the hemoglobinopathy.

In one embodiment of this aspect and all other aspects provided herein, the hemoglobinopathy is sickle cell anemia.

In another embodiment of this aspect and all other aspects provided herein, the at least one symptom of the hemoglobinopathy comprises the presence or number of sickle cells in a blood sample obtained from the subject.

In another embodiment of this aspect and all other aspects provided herein, the percentage of fetal hemoglobin in a blood sample obtained from the subject is increased following treatment of the subject with the vector.

Another aspect provided herein relates to a method for increasing the amount or percentage of fetal hemoglobin in a subject, the method comprising administering a nucleic acid vector or an RNA transcript as described herein to a cell, thereby increasing the amount or percentage of fetal hemoglobin in the cell.

In one embodiment of this aspect and all other aspects provided herein, the amount or percentage of fetal hemoglobin in a subject is increased by at least 5%.

Another aspect provided herein relates to a cell comprising a nucleic acid vector as described herein.

Also provided herein, in another aspect, is a cell expressing or comprising an RNA transcript as described herein.

In some embodiments, the shmiR constructs can be as described in US2021/0085707 or US2017/0218372, the contents of each of which are incorporated herein by reference in their entirety.

### BRIEF DESCRIPTION OF THE DRAWINGS

FIGS. 1A-1C. Outline of double shmiR vector gene therapy experiment. (FIG. 1A) The shmiR target BCL11A is in miR223 scaffold, while shmiR target ZNF410 is in miR144 scaffold that is highly expressed in erythroid cells. The two shmiRs are expressed under control of the β-globin promoter and regulatory elements derived from hypersensitive sites 2 and 3 (HS2 and HS3) of the human β-globin locus control region (LCR). (FIG. 1B) Outline of in vitro CD34+ transduction and erythroid differentiation. Healthy or patient CD34+ were transduced by lentivirus then differentiated in a three phase erythroid culture. Transduction efficiency and target gene expression were analyzed at day 11, and hemoglobin expression and disease phenotypes were analyzed at day 18. (FIG. 1C) Outline of in vivo transplant experiment in SCD mouse model. Lineage-depleted (Lin (−)) bone marrow (BM) cells derived from homozygous βS/βS mice were transduced by lentivirus with double shmiR. One million transduced cells were delivered by retro-orbital injection after recipient mice (Pep B6 [CD45.2 congenic]) were lethally irradiated. Peripheral blood was collected at weeks 4 (W4), 8 (W8), 12 (W12) and 16 (W16) to measure engraftment and RBC indices. BM cells were collected at 16 (W16) to measure engraftment, transduction efficiency, and expression.

FIGS. 2A-2D. Efficient knockdown of BCL11A and ZNF410 by double shmiR vector leads to high γ-globin and HbF induction in erythroid cells differentiated in vitro from transduced human CD34+ HSPCs. (FIG. 2A) BCL11A and ZNF410 mRNA expression as measured by RT-qPCR with GAPDH as control on day 11 of differentiation. (FIG. 2B) BCL11A and ZNF410 protein expression as measured by western blot with GAPDH as a loading control. (FIG. 2C) Induction of γ-globin mRNA as determined by RT-qPCR on day 18 of differentiation. (FIG. 2D) Hemoglobin F of cell lysates as measured by HPLC on day 18 of differentiation.

FIGS. 3A-3D. Efficient knockdown of BCL11A and ZNF410 by double shmiR vector in erythrocytes differentiated in vitro from transduced SCD patient CD34+ HSPCs. (FIGS. 2A-2D. (FIG. 3A) BCL11A and ZNF410 mRNA expression was measured by RT-qPCR with GAPDH as control on day 11 of differentiation. (FIG. 3B) Hemoglobin F of cell lysates was measured by HPLC on day 18 of differentiation. Phase-contrast microscope image of representative sample of Hoechst 33342-negative sorted enucleated erythroid progeny 30 min after sodium MBS treatment of erythrocytes differentiated from untransduced or transduced SCD CD34+ HSPCs; white arrows indicate sickle forms, scale bar, 50 μm. (FIG. 3C) Micropgraphs showing the effects of treatment on sickling of cells. (FIG. 3D) Quantification of sickled cells from untransduced and transduced enucleated erythroid cells at 30 min after MBS treatment.

FIGS. 4A-4D. Efficient knockdown of BCL11A and ZNF410 by double shmiR vector in erythrocytes differentiated in vitro from β-thalassemia patient CD34+ HSPCs. (FIG. 4A) BCL11A and ZNF410 mRNA expression was measured by RT-qPCR with GAPDH as control on day 11 of differentiation. (FIG. 4B) Induction of globin mRNA was determined on day 18 of differentiation by RT-qPCR. (FIG. 4C) Hemoglobin of cell lysates was measured by HPLC on day 18 of differentiation. (FIG. 4D) Cell size by relative forward scatter intensity of enucleated erythroid cells, normalized to healthy donor.

FIGS. 5A-5F. Analysis of in vivo hematologic parameters of Berkeley-SCD mouse model. Mice were bled at weeks 4, 16, and 20 and peripheral blood (PB) was analyzed. (FIG. 5A) Red blood cell (RBC) count, (FIG. 5B) reticulocyte counts. (FIG. 5C) Percentage of sickled red blood cells in PB after MBS treatment were quantitated. (FIG. 5D) Representative FACS plots of peripheral blood cells stained for the erythroid differentiation markers CD71 and Ter119 at week 16. Whole bone marrow (BM) and spleen was taken from each mouse at week 16 and analyzed. (FIG. 5E) Percentages γ-globin mRNA expression of erythroid cells in BM determined by RT-qPCR. (FIG. 5F) Spleen weights at week 16.

FIGS. 6A-6I. Efficient knockdown of BCL11A and ZNF410 by double shmiR vector leads to high γ-globin and HbF induction in erythroid cells differentiated in vitro from transduced human CD34+ HSPCs. (FIG. 6A) Schematic of virus transduction and erythroid differentiation of human CD34+ HSPCs. (FIG. 6B) BCL11A and ZNF410 mRNA expression as measured by RT-qPCR with GAPDH as control on day 11 of differentiation. (FIG. 6C) BCL11A and ZNF410 protein expression as measured by western blot with GAPDH as a loading control. (FIG. 6D) Induction of γ-globin mRNA as determined by RT-qPCR on day 18 of differentiation. (FIG. 6E) Hemoglobin F of cell lysates as measured by HPLC on day 18 of differentiation. (FIG. 6F) Correlation of γ-globin determined by RT-qPCR versus HbF by HPLC. Black dots represent samples transduced with different shmiR vectors. The Pearson correlation coefficient (r²) is shown. (FIG. 6G) Differentiation status of erythroid cells after 18 days in culture using CD71 and CD235a. (FIG. 611) Summary of the data shown in G normalized to the percentage CD71-CD235a+ population. (FIG. 6I) Enucleation of in vitro differentiated erythroid cells. Data represent mean±SD, n=3. ns, not significant, *P<0.05.

FIGS. 7A-7G. Efficient knockdown of BCL11A and ZNF410 by double shmiR vector in erythrocytes differentiated in vitro from transduced SCD patient CD34+ HSPCs. (FIG. 7A) BCL11A and ZNF410 mRNA expression was measured by RT-qPCR with GAPDH as control on day 11 of differentiation. Data represent mean±SD, n=3. (FIG. 7B) Induction of γ-globin mRNA was determined on day 18 of differentiation by RT-qPCR. Data represent mean±SD, n=3, *P<0.05. (FIG. 7C) Hemoglobin F of cell lysates was measured by HPLC on day 18 of differentiation. Data represent mean±SD, n=3. *P<0.05. (FIG. 7D) Cell proliferation of shmiR transduced cells during erythroid differentiation. Data represent mean±SD, n=3. (FIG. 7E) Phase-contrast microscope image of representative sample of Hoechst 33342-negative sorted enucleated erythroid progeny 30 min after sodium MBS treatment from erythrocytes differentiated from nontransduced or transduced SCD CD34+ HSPCs; arrows indicate sickle forms, scale bar, 50 μm. (FIG. 7F) Quantification of sickled cells from nontransduced and transduced enucleated erythroid cells at 30 min after MBS treatment. Data are plotted as mean, symbols indicate different SCD patients, and each data point represents an independent replicate. **P<0.01. (FIG. 7G) Correlation of HbF expression assessed by HPLC versus numbers of sickled cells. Black dots represent samples transduced with different shmiR vectors or nontransduced. Correlation coefficient (r²) is shown for all data.

FIGS. 8A-8D. Efficient knockdown of BCL11A and ZNF410 by double shmiR vector in erythrocytes differentiated in vitro from β-thalassemia patient CD34+ HSPCs. (FIG. 8A) BCL11A and ZNF410 mRNA expression was measured by RT-qPCR with GAPDH as control on day 11 of differentiation. (FIG. 8B) Induction of globin mRNA was determined on day 18 of differentiation by RT-qPCR. (FIG. 8C) Hemoglobin of cell lysates was measured by HPLC on day 18 of differentiation. (FIG. 8D) Cell size by relative forward scatter intensity of enucleated erythroid cells, normalized to healthy donor. Data represent mean±SD, n=3, **P<0.01; *P<0.05.

FIGS. 9A-9H. Hematopoietic reconstitution of transduced human CD34+ HSPCs in immunodeficient NBSGW mice. Animals were euthanized 16 weeks after transplantation, and bone marrow was collected and sorted into various subpopulations by flow cytometry. (FIG. 9A) Human chimerism in the bone marrow of transplanted animals as determined by expression of hCD45. Data represent mean±SD, each dot represents one animal. (FIG. 9B) Lineage distribution of human CD45 positive bone marrow (BM) cells, data represent mean±SD. (FIG. 9C) Vector copy number (VCN) determined in BM and multilineage cells, data represent mean±SD, each dot represents one animal. Gene expression analysis by RT-qPCR in human cells from BM of engrafted mice. BCL11A and ZNF410 expression normalized to GAPDH in human B cells (FIG. 9D) or human erythroid cells (FIG. 9E) sorted from BM of engrafted mice, and γ-globin expression (FIG. 9F) by RT-qPCR in human erythroid cells sorted from BM. Data represent mean±SD. Each data point represents an individual mouse. ns, not significant, ***P<0.001; *P<0.05. (FIG. 9G) γ-globin shown in relation to VCN for SS BCL11A and DS BCL11A/ZNF410. (FIG. 9H) HbF by HPLC analysis of CD34+ cells isolated from the bone marrow and subjected to in vitro erythroid differentiation. Data represent mean±SD. Each data point represents an individual mouse. ns, not significant, ***P<0.001; **P<0.01 *P<0.05.

FIGS. 10A-10H. Correction of in vivo hematologic parameters in Berkeley-SCD mouse model. Mice were bled at weeks 4, 16, and 20 and peripheral blood (PB) was analyzed. (FIG. 10A) PB engraftment in transplanted mice. (FIG. 10B) Red blood cell (RBC) count, (FIG. 10C) percentage of hematocrit (HCT), (FIG. 10D) hemoglobin (HGB) level, (FIG. 10E) reticulocyte counts. (FIG. 10F) Percentage of sickled red blood cells in PB after MBS treatment were quantitated. (FIG. 10G) Representative FACS plots of peripheral blood cells stained for the erythroid differentiation markers CD71 and Ter119 at week 16. (FIG. 10H) Summary of the percentages of CD71+Ter119+high erythroid precursor cell population. Data represent mean±SD. Symbols indicate mice transplanted with different shmiR vectors or nontransduced cells, each data point represents an individual mouse. ns, not significant, ***P<0.001; **P<0.01 *P<0.05.

FIGS. 11A-11E. HbF expression in vivo in BM cells from Berkeley-SCD mouse model. Whole BM was taken from each mouse at week 16 and analyzed. (FIG. 11A) BM engraftment in transplanted mice. (FIG. 11B) Vector copy number (VCN) in BM was determined. (FIG. 11C) Percentages γ-globin mRNA expression of erythroid cells in BM determined by RT-qPCR. (FIG. 11D) Correlation of γ-globin expression assessed by RT-qPCR in erythroid cells of BM versus sickled cell in PB after MBS treatment, correlation coefficient (r²) is shown for all data. (FIG. 11E) Spleen weights at week 16. Data represent mean±SD. Each data point represents an individual mouse. ns, not significant, ***P<0.001; **P<0.01 *P<0.05

FIGS. 12A-12F. Identification of efficient ZNF410 shmiR for HbF induction. (FIG. 12A) Schematic representation of LV-SFFV-miR144 ZNF410 lentiviral vectors with shRNA targeting ZNF410. (FIG. 12B) Schema of virus transduction and erythroid differentiation of human CD34+ HSPCs. (FIG. 12C) Comparison of knockdown efficiency of ZNF410 shmiRs (labeled as 1, 1M, 2, 2M, 3 and 3M). ZNF410 expression at day 11 by RT-qPCR analysis of erythrocytes differentiated from ZNF410 shmiRs-transduced CD34+ HSPCs. Data represent mean±SD, n=3. (FIG. 12D) Correlation between the degree of knockdown of ZNF410 and induction of γ-globin in erythroid cells differentiated in vitro from transduced hCD34+ cells. Black dots represent samples transduced with different ZNF410 shmiR vectors, the Pearson correlation coefficient (r²) is shown. (FIG. 12E) Induction of γ-globin mRNA by RT-qPCR determined on day 18 of differentiation. Data represent mean±SD, n=3. (FIG. 12F) Hemoglobin F in cell lysates measured by HPLC on day 18 of differentiation. Data represent mean±SD, n=3.

FIGS. 13A-13C. Lentiviral vectors used for transduction. (FIG. 13A) LV-LCR-miR144 ZNF410 vector (SS ZNF410), top; (FIG. 13B) LV-LCR-miR223 BCL11A vector (SS BCL11A), middle; and (FIG. 13C) double shmiR vector LV-LCR-miR223 BCL11A-miR144 ZNF410 (DS BCL11A/ZNF410), bottom.

FIG. 14. Gene marking of in vitro differentiated cells after transduction of human CD34+ HSPCs with shmiR vector as percent fluorescent+ cells. Data represent mean±SD, n=3, each data point represents an independent replicate.

FIG. 15. HPLC chromatograms of cell lysates obtained after 18 days of erythroid differentiation of transduced human CD34+ HSPCs. The arrow indicates the HbF peaks and the percentage of HbF of total hemoglobin is shown below the chromatogram.

FIG. 16. Gene marking of SCD patient shmiR vector transduced CD34+ HSPCs differentiated in vitro as a percentage of Venus+ cells. Data represent mean±SD, n=3, each data point represents an independent replicate.

FIGS. 17A-17B. Effect of shmiR expression on in vitro erythroid differentiation and enucleation of SCD patient transduced CD34+ HSPCs. (FIG. 17A) Percentage of CD71-CD235a+ erythroid cells after 18 days in culture. (FIG. 17B) Enucleation of differentiated erythroid cells. Each data point represents an individual sample. Data represent mean±SD, n=3.

FIG. 18. HPLC chromatograms of cell lysates obtained on 18 days of erythroid differentiation of transduced SCD patient CD34+ HSPCs. The arrow indicates the HbF peaks and the percentage of HbF of total hemoglobin is shown below the chromatogram.

FIG. 19. Gene marking of shmiR vector transduced β-thalassemia patient CD34+ HSPCs differentiated in vitro as a percentage of Venus+ cells. Data represent mean±SD, n=3, each data point represents an independent replicate.

FIGS. 20A-20B. Effect of shmiR vector expression on in vitro erythroid differentiation and enucleation of transduced β-thalassemia patient CD34+ HSPCs. (FIG. 20A) Percentage of CD71-CD235a+ erythroid cells after 18 days in culture. (FIG. 20B) Enucleation of erythroid cells. Data represent mean±SD, n=3.

FIGS. 21A-21F. Peripheral blood (PB) of transplanted mice NBSGW mice analyzed at weeks 4, 8, 12 and 16. (FIG. 21A) Human chimerism in the PB of transplanted animals. Peripheral blood hematological parameters of transplanted animals were analyzed, including (FIG. 21B) white blood cell (WBC), (FIG. 21C) red blood cell (RBC), (FIG. 21D) platelet count (PLT), (FIG. 21E) hematocrit (HCT), (FIG. 21F) hemoglobin (HGB). Data represent mean±SD, each data point represents an individual mouse. ns, not significant.

FIGS. 22A-22B. Gene expression analysis by RT-qPCR in human cells from BM of engrafted mice. BCL11A and ZNF410 expression normalized to GAPDH in human myeloid cells (FIG. 22A) and human CD34+ cells (FIG. 22B) sorted from BM of engrafted mice. Data represent mean±SD. ns, not significant.

FIG. 23. HbF shown in relation to VCN for SS BCL11A and DS BCL11A/ZNF410. Each data point represents an individual mouse.

FIGS. 24A-24B. Efficient knockdown of Zfp410 by SS Zfp410 shmiR vector leads to high Hbb-γ induction in erythroid differentiated MEL cells in vitro. Zfp410 (FIG. 24A) and Hbb-γ (FIG. 24B) mRNA expression as measured by RT-qPCR with Gapdh as control. Data represent mean±SD.

FIG. 25. VCN of gene modified SCD Lin⁻ BM cells in vitro before transplant by CFU. Data represent mean±SD.

FIG. 26. γ-globin of erythroid cells shown in relation to VCN for SS BCL11A and DS BCL11A/Zfp410. Each data point represents an individual mouse.

FIG. 27 Schematic representation of multiple shmiR and double shmiR sequences.

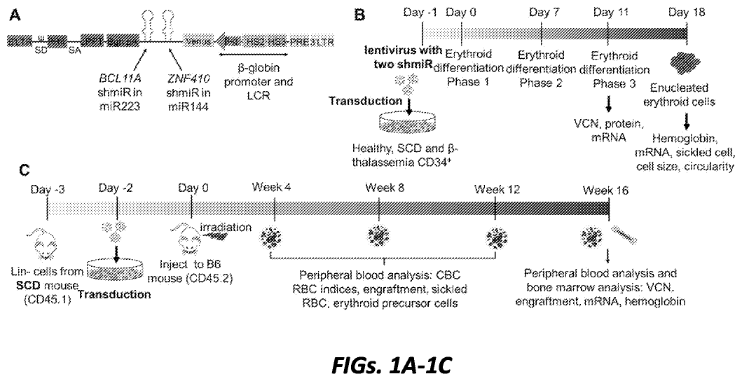

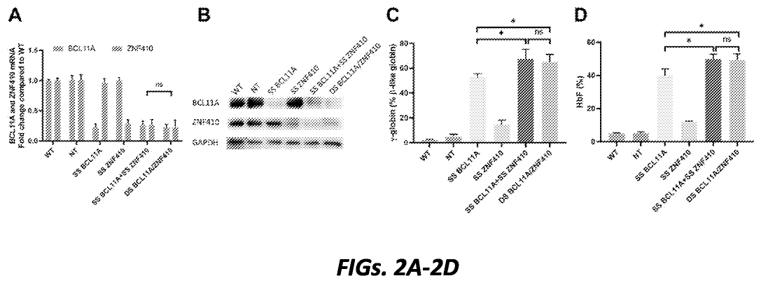

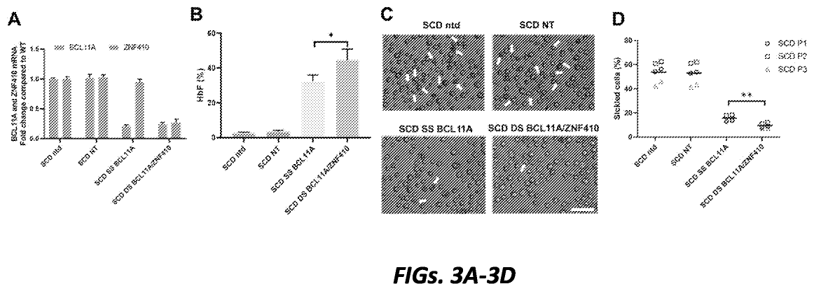

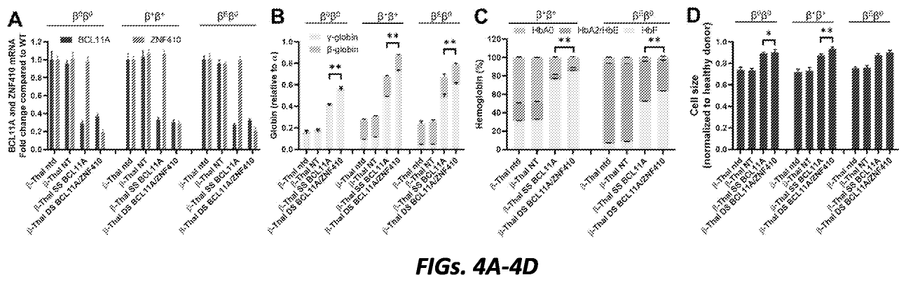

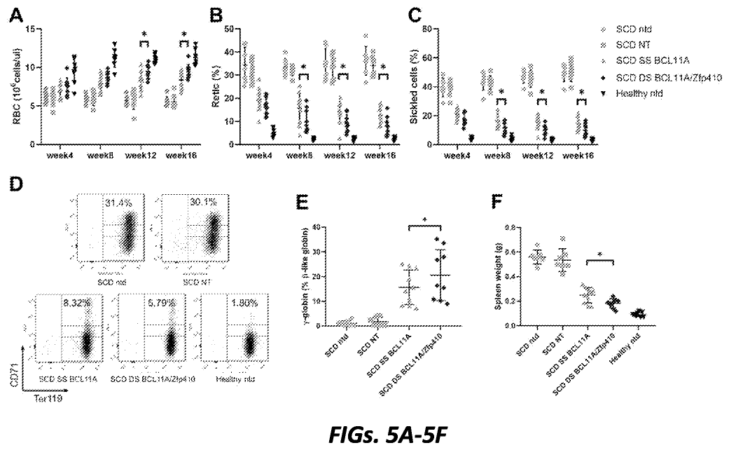

### DETAILED DESCRIPTION

The disclosure described herein is based, in part, on development of a construct comprising at least two tandem microRNA-adapted short hairpin RNAs, which can be expressed in the progeny of hematopoietic stem cells (HSC) as a therapeutic treatment for a hemoglobin disorder. The nucleic acids encoding each of the short hairpin RNAs are placed within a different miRNA nucleic acid framework in the construct in order to permit simultaneous delivery of the two short hairpin RNAs in miRNA frameworks (shmiRs); the miRNA frameworks are designed such that the tandem microRNA-adapted short hairpin RNA-encoding sequences are different so as not to permit or undergo homologous recombination in the construct, thereby ensuring the integrity of the construct and the treatment. In some embodiments, the short hairpin RNAs each comprise a region that binds to and targets a different gene to induce the expression of fetal hemoglobin (e.g., γ-hemoglobin). Such tandem constructs can be used in the treatment of hemoglobinopathies, including sickle cell disease (SCD) and thalassemia (THAL), by induction of γ-globin via inhibition of, for example, the BCL11A gene product and either a ZNF410 or ZBTB7A gene product.

### Definitions

As used herein, the terms “shRNA embedded miRNA,” and “shmiR” are used interchangeably and refer to an shRNA whose sense and antisense strands are embedded into an miRNA scaffold, which retains the miRNA flanking regions and loop. For example, in one embodiment, the skilled artisan can design a short hairpin RNA expressed from an miR-223 primary transcript or an miR144 primary transcript. This design adds a Drosha processing site to the shRNA construct and has been shown to greatly increase knockdown efficiency (Pusch et al., 2004). In particular embodiments, the hairpin stem of a shmiR can comprise 21-nt of dsRNA and a 15-nt loop from a human miRNA. Adding the miRNA loop and flanking sequences on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins. In preferred embodiments, a shmiR comprises a 21-nt guide strand, wherein about 17-nt correspond to an antisense RNA that binds a target mRNA and about 4-nt correspond to GC-rich sequences, e.g., GCGC, that improve 3′-end thermodynamic stability in the RNA duplex and promotes preferential RISC loading of the intended guide strand. In one embodiment, the polynucleotide encodes a shmiR.

As used herein, the term “tandem shmiR” refers to an RNA transcript comprising at least two shmiRs in tandem, wherein each of the shmiRs comprises the framework of a different miRNA (i.e., a first and second miRNA). In one embodiment, the first and second shmiR do not undergo homologous recombination. When there are two tandem shmiRs in a construct, the construct is then called a “double shmiR.” A “triple shmiR” comprises three tandem shmiRs and so forth.

As used herein, the term “tandem shmiR construct” refers to a nucleic acid sequence, such as an expression cassette, comprising or encoding a tandem shmiR RNA transcript. Such expression cassettes can be incorporated into a vector for delivery to a cell or subject.

As used herein, the terms “shRNA” or “short hairpin RNA” refer to double-stranded structure that is formed by a single self-complementary RNA strand. The shRNA is processed intracellularly to generate a short interfering RNA (siRNA).

As used herein, the terms “miRNA” or “microRNA” refer to small non-coding RNAs of 20-22 nucleotides, typically excised from 70 nucleotide foldback RNA precursor structures known as pre-miRNAs. miRNAs negatively regulate their targets in one of two ways depending on the degree of complementarity between the miRNA and the target. First, miRNAs that bind with perfect or nearly perfect complementarity to protein-coding mRNA sequences induce the RNA-mediated interference (RNAi) pathway. miRNAs that exert their regulatory effects by binding to imperfect complementary sites within the 3′ untranslated regions (UTRs) of their mRNA targets, repress target-gene expression post-transcriptionally, apparently at the level of translation, through a RISC complex that is similar to, or possibly identical with, the one that is used for the RNAi pathway. Consistent with translational control, miRNAs that use this mechanism reduce the protein levels of their target genes, but the mRNA levels of these genes are only minimally affected.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

A “nucleic acid,” as described herein, can be RNA or DNA, and can be single or double stranded, and can be selected, for example, from a group including: nucleic acid encoding a protein of interest, oligonucleotides, nucleic acid analogues, for example peptide-nucleic acid (PNA), pseudo-complementary PNA (pc-PNA), and locked nucleic acid (LNA). Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, microRNAi (miRNA), and antisense oligonucleotides.

The term “nucleic acid cassette” or “expression cassette” as used herein refers to genetic sequences within an expression vector which can express an RNA transcript. In one embodiment, the nucleic acid cassette comprises a polynucleotide(s)-of-interest. In another embodiment, the nucleic acid cassette contains one or more expression control sequences, e.g., a promoter, enhancer, poly(A) sequence, and a polynucleotide(s)-of-interest. The nucleic acid cassette is positionally and sequentially oriented within the vector such that the nucleic acid in the cassette can be transcribed into RNA. Preferably, the cassette has its 3′ and 5′ ends adapted for ready insertion into a vector, e.g., it has restriction endonuclease sites at each end. In a preferred embodiment, the nucleic acid cassette one or more expression control sequences operably linked to a polynucleotide encoding a therapeutic RNA, e.g., a shmiR, that can be used to treat, prevent, or ameliorate a genetic disorder. The cassette can be removed and inserted into a plasmid or viral vector as a single unit.

The term “vector” is used herein to refer to a nucleic acid molecule that comprises a nucleic acid sequence encoding an RNA transcript, such as a double or tandem shmiR. A vector can include sequences that direct autonomous replication in a cell, or can include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., lentiviral vectors.

The term “lentiviral vector” refers to a retroviral vector or plasmid containing structural and functional genetic elements, or portions thereof, including LTRs that are primarily derived from a lentivirus. The terms “lentiviral vector” and “lentiviral expression vector” may be used to refer to lentiviral transfer plasmids and/or infectious lentiviral particles in particular embodiments. Where reference is made herein to elements such as cloning sites, promoters, regulatory elements, heterologous nucleic acids, etc., it is to be understood that the sequences of these elements are present in RNA form in the lentiviral particles contemplated herein and are present in DNA form in the DNA plasmids contemplated herein.

As used herein, “treating” or “reducing a risk of developing a hemoglobinopathy” in a subject refers to the reduction or amelioration of at least one symptom of a given hemoglobinopathy. In one embodiment, “treating” is used to refer to the reduction in severity or progression of, a hemoglobinopathy in a subject. In another aspect, the methods can also be used to reduce a risk of developing a hemoglobinopathy in a subject, delaying the onset of symptoms of a hemoglobinopathy in a subject (e.g., pain), or increasing the longevity of a subject having a hemoglobinopathy. In one aspect, the methods can include selecting a subject on the basis that they have, or are at risk of developing, a hemoglobinopathy, but do not yet have a hemoglobinopathy, or a subject with an underlying hemoglobinopathy. Selection of a subject can include the presence of visible symptoms of a hemoglobinopathy or via a blood test, genetic testing, or clinical recordings. If the results of the test(s) indicate that the subject has a hemoglobinopathy, the methods also include administering the compositions described herein, thereby treating, or reducing the risk of developing, a hemoglobinopathy in the subject. For example, a subject that has been diagnosed with SCD can comprise a genotype HbSS, HbS/β0 thalassemia, HbSD, or HbSO, and/or HbF<10% by electrophoresis.

As used herein, the term “hemoglobinopathy” refers to a condition involving the presence of an abnormal hemoglobin molecule in the blood. Examples of hemoglobinopathies include, but are not limited to, SCD and THAL. Also included are hemoglobinopathies in which a combination of abnormal hemoglobins is present in the blood (e.g., sickle cell/Hb-C disease). An example of such a disease includes, but is not limited to, SCD and THAL. SCD and THAL and their symptoms are well-known in the art and are described in further detail below. Subjects can be diagnosed as having a hemoglobinopathy by a health care provider, medical caregiver, physician, nurse, family member, or acquaintance, who recognizes, appreciates, acknowledges, determines, concludes, opines, or decides that the subject has a hemoglobinopathy.

The term “sickle cell disease” or “SCD” is defined herein to include any symptomatic anemic condition which results from sickling of red blood cells. Manifestations or symptoms of SCD can include: anemia; pain; and/or organ dysfunction, such as renal failure, retinopathy, acute-chest syndrome, ischemia, priapism, and stroke. As used herein the term “SCD” refers to a variety of clinical problems attendant upon SCD, especially in those subjects who are homozygotes for the sickle cell substitution in HbS. Additional symptoms of SCD include a delay of growth and development, an increased tendency to develop serious infections (particularly due to pneumococcus), and a marked impairment of splenic function preventing effective clearance of circulating bacteria, with recurrent infarcts and eventual destruction of splenic tissue. Also included in the term “SCD” are acute episodes of musculoskeletal pain, which affect primarily the lumbar spine, abdomen, and femoral shaft, and which are similar in mechanism and in severity. In adults, such attacks commonly manifest as mild or moderate bouts of short duration every few weeks or months interspersed with agonizing attacks lasting 5 to 7 days that strike on average about once a year. Among events known to trigger such crises are acidosis, hypoxia, and dehydration, all of which potentiate intracellular polymerization of HbS (J. H. Jandl, Blood: Textbook of Hematology, 2nd Ed., Little, Brown and Company, Boston, 1996, pages 544-545).

As used herein, a “thalassemia” or “THAL” refers to a hereditary disorder characterized by defective production of hemoglobin. In one embodiment, the term encompasses hereditary anemias that occur due to mutations affecting the synthesis of hemoglobins. In other embodiments, the term includes any symptomatic anemia resulting from thalassemic conditions such as severe or β-thalassemia, thalassemia major, thalassemia intermedia, and α-thalassemias such as hemoglobin H disease. β-thalassemias are caused by a mutation in the β-globin chain, and can occur in a major or minor form. In the major form of β-thalassemia, children are normal at birth, but develop anemia during the first year of life. The mild form of β-thalassemia produces small red blood cells. Alpha-thalassemias are caused by deletion of a gene or genes from the globin chain.

By the phrase “risk of developing disease” is meant the relative probability that a subject will develop a hemoglobinopathy in the future as compared to a control subject or population (e.g., a healthy subject or population). For example, an individual carrying the genetic mutation associated with SCD, an A to T mutation of the β-globin gene, and whether the individual in heterozygous or homozygous for that mutation increases that individual's risk.

A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches.

A “therapeutically effective amount” refers to an amount effective, at dosages and for periods of time necessary, to achieve the desired therapeutic result, such as treatment or prevention of a hemoglobinopathic condition or reduction of at least one symptom of a hemoglobinopathy to be treated. A therapeutically effective amount of a plasmid or vector (e.g., lentiviral vector) encoding tandem shmiRs can vary according to factors such as the disease state, age, sex, and weight of the individual, and the ability of the shmiRs to elicit a desired response in the individual. Dosage regimens can be adjusted to provide the optimum therapeutic response. A therapeutically effective amount is also one in which any toxic or detrimental effects of the vector are outweighed by the therapeutically beneficial effects. For example, the potential toxicity of lentiviral vectors for use with the constructs described herein can be assayed using cell-based assays or art recognized animal models and a therapeutically effective modulator can be selected which does not exhibit significant toxicity. In a preferred embodiment, a therapeutically effective amount of a nucleic acid construct encoding tandem shmiRs is sufficient to treat a hemoglobinopathy.

As used herein, the term “increasing fetal hemoglobin levels” in a cell or subject indicates that HbF is at least 5% higher in cell populations or in a biological sample obtained from a subject treated with a tandem shmiR construct, than in a comparable, control population or subject, wherein no tandem shmiR construct is present. In some embodiments, the percentage of HbF expression in a tandem shmiR construct treated population or subject is at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher, or more than a control treated population of comparable size and culture conditions or as compared to the level of fetal hemoglobin in a given subject prior to treatment with the tandem shmiR construct. The term “control treated cell population” is used herein to describe a population of cells that has been treated with identical media, viral induction, nucleic acid sequences, temperature, confluency, flask size, pH, etc., with the exception of a tandem shmiR construct.

A “subject,” as used herein, includes any animal that exhibits a symptom of a disease, disorder, or condition that can be treated with the gene therapy vectors, cell-based therapeutics, and methods disclosed elsewhere herein. In preferred embodiments, a subject includes any animal that exhibits symptoms of a disease, disorder, or condition of the hematopoietic system, e.g., a hemoglobinopathy, that can be treated with the gene therapy vectors, cell-based therapeutics, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include animals that exhibit aberrant amounts (lower or higher amounts than a “normal” or “healthy” subject) of one or more physiological activities that can be modulated by gene therapy.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### Hemoglobin Isoforms

Normal adult hemoglobin comprises four globin proteins, two of which are alpha (α) proteins and two of which are beta (β) proteins. During mammalian fetal development, particularly in humans, the fetus produces fetal hemoglobin, which comprises two gamma (γ)-globin proteins instead of the two β-globin proteins. At some point during fetal development, a globin fetal switch occurs at which point erythrocytes in the switch from making predominantly γ-globin to making predominantly β-globin. The developmental switch from production of predominantly fetal hemoglobin or HbF (a2γ2) to production of adult hemoglobin or HbA (α2β2) begins at about 28 to 34 weeks of gestation and continues until 6-12 months post-natal. This switch results primarily from decreased transcription of the γ-globin genes and increased transcription of β-globin genes. On average, the blood of a normal adult contains only about 2% HbF, though residual HbF levels have a variance of over 20-fold in healthy adults (Atweh, Semin. Hematol. 38:367-73 (2001)).

The methods and compositions described herein can be used to induce the expression of fetal hemoglobin in adult subjects to overcome the deficiencies of adult hemoglobin isoforms in subjects having hemoglobinopathies, such as sickle cell disease.

### β-Hemoglobinopathies

β-hemoglobinopathies encompass a number of anemias of genetic origin in which there is a decreased production and/or increased destruction (hemolysis) of red blood cells (RBCs). These also include genetic defects that result in the production of abnormal hemoglobins (such as sickle hemoglobin) leading to abnormal polymerization of the sickle globin molecules with a propensity to damage the red cell membrane, lead to vessel occlusion and a concomitant impaired ability to maintain oxygen concentration. Some such disorders involve the failure to produce normal β-globin in sufficient amounts (e.g., β-thalassemias), while others involve the failure to produce normal β-globin entirely. This leads to an imbalance of alpha and beta chains, damage and premature destruction of the red blood cells.

The search for treatment aimed at reduction of sickle globin polymerization and globin chain imbalance in patients with β-hemoglobinopathies has focused on the pharmacologic manipulation of fetal HbF. The therapeutic potential of such approaches is demonstrated by observations that certain populations of adult patients with R chain abnormalities and higher than normal levels of HbF experience a milder clinical course of disease than patients with normal adult levels of HbF.

The tandem shmiRs described herein can be used to manipulate the levels of fetal hemoglobin to compensate for defective adult hemoglobin proteins in subjects with a hemoglobinopathy. The transcriptional repressor BCL11A has been successfully used as a therapeutic target for the treatment of β-hemoglobinopathies and as such, constructs comprising tandem shmiRs that target BCL11A are described herein. Also provided herein are constructs comprising tandem shmiRs that target ZNF410 and/or ZBTB7A.

BCL11A is a validated therapeutic target for reactivation of γ-globin gene and therefore HbF expression in the major hemoglobinopathies, sickle cell disease (SCD) and β-thalassemias. Down modulation or genetic deletion of BCL11A relieves γ-globin repression and inactivation of BCL11A in the erythroid lineage prevents SCD phenotype and organ toxicities in genetically engineered mice. The mouse embryonic Hbb-y gene is a functional homolog of the human γ-globin gene, and therefore serves as a convenient surrogate for assessment of the effect of BCL11A knockdown in murine erythroleukemia (MEL) cells.

### Hematopoietic Cells

The tandem shmiR constructs described herein can be used to induce expression of fetal hemoglobin in hematopoietic cells. In some embodiments, the tandem shmiR construct is administered in vivo, but treatment of isolated hematopoietic stem cells or mature cells ex vivo or in vitro for administration of therapeutic cells to a subject in need thereof is also contemplated herein.

Mature hematopoietic blood cells have a finite lifespan and must be continuously replaced throughout life. Blood cells are produced by the proliferation and differentiation of a very small population of pluripotent hematopoietic stem cells (HSCs) that also have the ability to replenish themselves by self-renewal. HSCs are multipotent, self-renewing progenitor cells that develop from mesodermal hemangioblast cells. HSCs are the blood cells that give rise to all the other blood cells, that includes all the differentiated blood cells from the erythroid, lymphoid and myeloid lineages. HSCs are located in the adult bone marrow, peripheral blood, and umbilical cord blood.

During differentiation, the progeny of HSCs progress through various intermediate maturational stages, generating multi-potential hematopoietic progenitor cells and lineage-committed hematopoietic progenitor cells, prior to reaching maturity. Bone marrow (BM) is the major site of hematopoiesis in humans and, under normal conditions, only small numbers of HSCs and hematopoietic progenitor cells can be found in the peripheral blood (PB). Treatment with cytokines (in particular granulocyte colony-stimulating factor; G-CSF), myelosuppressive drugs used in cancer treatment, and compounds that disrupt the interaction between hematopoietic cells and BM stromal cells can rapidly mobilize large numbers of stem and progenitor cells into the circulation.

As used herein, the term “hematopoietic progenitor cell” as the term is used herein, refers to cells of a hematopoietic stem cell lineage that give rise to all the blood cell types including the myeloid (monocytes and macrophages, neutrophils, basophils, eosinophils, erythrocytes, megakaryocytes/platelets, dendritic cells), and the lymphoid lineages (T-cells, B-cells, NK-cells). A “cell of the erythroid lineage” indicates that the cell being contacted is a cell that undergoes erythropoeisis such that upon final differentiation it forms an erythrocyte or red blood cell (RBC). Such cells belong to one of three lineages, erythroid, lymphoid, and myeloid, originating from bone marrow hematopoietic progenitor cells. Upon exposure to specific growth factors and other components of the hematopoietic microenvironment, hematopoietic progenitor cells can mature through a series of intermediate differentiation cellular types, all intermediates of the erythroid lineage, into RBCs. Thus, cells of the “erythroid lineage,” as the term is used herein, comprise hematopoietic progenitor cells, rubriblasts, prorubricytes, erythroblasts, metarubricytes, reticulocytes, and erythrocytes.

In some embodiments of any method described herein, the hematopoietic progenitor cell contacted with a construct comprising tandem shmiRs has at least one of the cell surface marker characteristic of hematopoietic progenitor cells: CD34+, CD59+, Thy1/CD90+, CD38^{1o/-}, and C-kit/CD117+. Preferably, the hematopoietic progenitor cells have several of these markers.

In some embodiments of any method described herein, the hematopoietic progenitor cells of the erythroid lineage contacted with a construct comprising tandem shmiRs have the cell surface marker characteristic of the erythroid lineage: CD71 and Ter119.

In some embodiments of any method described herein, the HSC contacted with a construct comprising tandem shmiRs has at least one of the cell surface marker characteristic of hematopoietic progenitor cells: CD34+, CD59+, Thy1/CD90+, CD38^{1o/-}, and C-kit/CD117+.

In one embodiment of any method described herein, the hematopoietic stem cell or hematopoietic progenitor cell for use with the methods and compositions described is first collected from peripheral blood, cord blood, chorionic villi, amniotic fluid, placental blood, or bone marrow. In another embodiment of any methods described herein, the embryonic stem cell, somatic stem cell, progenitor cell, or bone marrow cell is collected from peripheral blood, cord blood, chorionic villi, amniotic fluid, placental blood, or bone marrow. In other embodiments, the hematopoietic stem or progenitor cells are contacted by in vivo administration with a construct comprising tandem shmiRs.

Peripheral blood progenitor cells (PBPC) have become the preferred source of hematopoetic progenitor cells for allogeneic and autologous transplantation because of technical ease of collection and shorter time required for engraftment. Traditionally, granulocyte-colony stimulating factor (G-CSF) has been used to stimulate more PBPC and release of hematopoetic progenitor cells from the bone marrow. Although regimens using G-CSF usually succeed in collecting adequate numbers of PBPC from healthy donors, 5%-10% will mobilize stem cells poorly and may require multiple large volume apheresis or bone marrow harvesting. Thus, in some embodiments, the hematopoietic progenitor cells are isolated from peripheral blood progenitor cells prior to contacting them with a construct that comprises tandem shmiRs.

In some embodiments of any method described herein, the embryonic stem cell, somatic stem cell, progenitor cell, bone marrow cell, or hematopoietic progenitor cell, or HSC is selected for the CD34+ surface marker prior to the contacting.

Accordingly, in one embodiment of any method described herein, an isolated CD34+ embryonic stem cell, isolated CD34+ somatic stem cell, isolated CD34+ progenitor cell, isolated CD34+ bone marrow cell, isolated CD34+ hematopoietic progenitor cell, or isolated CD34+ HSC (or populations thereof) is contacted with the composition described herein or contacted with the virus or vector carrying a tandem shmiR as described herein.

In one embodiment of any methods described herein, the contacting is in vitro, ex vivo or in vivo. In one embodiment of any methods described herein, the contacting is repeated at least once. That is, after the initial first contacting of the embryonic stem cell, somatic stem cell, progenitor cell, bone marrow cell, or hematopoietic progenitor cell, or HSC with the composition described herein or contacting with the virus or vector carrying a nucleic acid molecule encoding tandem shmiRs, the cell is washed, and the washed cell is then contacted for a second time with the composition described herein or contacted with the virus or vector carrying a nucleic acid molecule comprising tandem shmiRs.

In other embodiments, contacting with a tandem shmiR construct can be repeated at least twice after the initial first contacting. In one embodiment of any methods described herein, after the contacting, the contacted embryonic stem cell, somatic stem cell, progenitor cell, bone marrow cell, or hematopoietic progenitor cell, or HSC can be cryopreserved prior to use, for example, for ex vivo expansion and/or implantation into a subject.

In one embodiment of any methods described herein, after the contacting, the contacted embryonic stem cell, somatic stem cell, progenitor cell, bone marrow cell, or hematopoietic progenitor cell, or HSC is culture expanded ex vivo prior to use, for example, for cryopreservation, and/or implantation/engraftment into a subject.

In one embodiment of any method described herein, after contacting the cells with a tandem shmiR construct, the contacted embryonic stem cell, somatic stem cell, progenitor cell, bone marrow cell, or hematopoietic progenitor cell, or HSC is differentiated in culture ex vivo prior to use, for example, cryopreservation, and/or implantation/engraftment into a subject.

miRNA Frameworks

The terms “microRNA” and “miRNA” as used herein refer to short, single-stranded RNA molecules approximately 21-23 nucleotides in length which are partially complementary to one or more mRNA molecules (target mRNAs). miRNAs can functionally down-regulate gene expression by inhibiting translation or by targeting the mRNA for degradation or deadenylation. MiRNAs base-pair with miRNA recognition elements (MREs) located on their mRNA targets, usually on the 3′-UTR, through a region called the ‘seed region’ which includes nucleotides 2-8 from the 5′-end of the miRNA. Matches between a miRNA and its target are generally asymmetrical. The complementarity of seven or more bases to the 5′-end miRNA has been found to be sufficient for regulation.

Physiologically, microRNAs play important roles in the regulation of target genes by binding to complementary regions of messenger transcripts to repress their translation or regulate degradation (Griffiths-Jones Nucleic Acids Research. 2006; 34, Database issue: D140-D144). Frequently, one miRNA can target multiple mRNAs and one mRNA can be regulated by multiple miRNAs targeting different regions of, for example, the 3′ UTR. Once bound to an mRNA, miRNA can modulate gene expression and protein production by affecting, e.g., mRNA translation and stability (Baek et al. Nature. 2008; 455:64; Selbach et al. Nature. 2008; 455:58; Ambros. Nature. 2004; 431: 350-355; Bartel. Cell. 2004; 116: 281-297; Cullen. Virus Research. 2004; 102: 3-9; He et al. Nat. Rev. Genet. 2004; 5: 522-531; and Ying et al. Gene. 2004; 342: 25-28).

The methods and compositions described herein utilize miRNA frameworks or regions to flank an shRNA directed against BCL11A, ZNF410 or ZBTB7A. The use of such miRNA frameworks permits exploitation of the microRNA-biogenesis pathway to generate shRNAs or siRNAs that target expression of the target gene (e.g., BCL11A, ZNF410 or ZBTB7A). Lentiviral transgenes are engineered to express shRNAs that mimic primary microRNAs (pri-miRNAs) and are sequentially processed by the endogenous Microprocessor and Dicer complexes to generate small interfering RNAs (siRNAs) or short hairpin RNAs (shRNAs) with sequence complementarity to the BCL11A, ZNF410 or ZBTB7A messenger RNA (mRNA).

As used herein, the term “miRNA framework regions” refers to nucleic acid sequences derived from an miRNA that can be placed upstream and/or downstream of the shRNA and/or in the loop region of an shRNA in the shmiR construct as that term is used herein. Such framework regions permit endogenous processing of the gene-targeting shRNAs or siRNAs by Microprocessor and Dicer complexes to permit the inhibition of gene expression of BCL11A, ZNF410 and/or ZBTB7A.

In one embodiment, the tandem shmiR construct comprises a first or second shmiR comprising an miR223 framework region upstream of the gene targeting sequence, downstream of the gene targeting sequence and/or within the loop region of the shmiR. In another embodiment, the tandem shmiR construct comprises a first or second shmiR comprising an miR144 framework region upstream of the gene targeting sequence, downstream of the gene targeting sequence and/or within the loop region of the shmiR. In one embodiment, the tandem shmiR construct comprises a first shmiR comprising an miR223 framework region upstream of the gene targeting sequence, downstream of the gene targeting sequence and/or within the loop region of the shmiR and a second shmiR comprising an miR144 framework region framework region upstream of the gene targeting sequence, downstream of the gene targeting sequence and/or within the loop region of the shmiR.

In one embodiment, the first segment of the miR223 miRNA comprises the sequence of: GATCTCACTTCCCCACAGAAGCTCTTGGCCTGGCCTCCTGCAGTGCCACGCT (SEQ ID NO: 1).

In one embodiment, the loop segment comprises a segment of miR223. In one embodiment of this aspect and all other aspects provided herein, the miR223 loop segment comprises the sequence of: CTCCATGTGGTAGAG (SEQ ID NO: 2).

In one embodiment, the second segment of the miR223 miRNA comprises the sequence

(SEQ ID NO: 3)
AGTGCGGCACATGCTTACCAGCTCTAGGCCAGGGCAGATGGGATAT
GACGAATGGACTGCCAGCTGGATACAAGGATGCTCACC.

It is also specifically contemplated that one or more of SEQ ID Nos: 1, 2, or 3 can be used in a single shmiR, for example, a shmiR can comprise SEQ ID NO. 1 and 2, SEQ ID NO: 1 and 3, SEQ ID NO: 2 and 3, or SEQ ID Nos: 1, 2, and 3, or any other combination thereof.

In one embodiment, the first segment of the miR144 miRNA comprises the sequence of: CGCTTTTCAAGCCATGCTTCCTGTGCCCCCAGTGGGGCCCTGGCTG (SEQ ID NO: 7).

In one embodiment, the loop segment comprises a segment of miR144. In one embodiment of this aspect and all other aspects provided herein, the miR144 loop segment comprises the sequence of AGTTTGCGATGAGACAC (SEQ ID NO: 8).

In one embodiment, the second segment of the miR144 miRNA comprises the sequence of: AGTCCGGGCACCCCCAGCTCTGGAGCCTGACAAGGAGGACAGGAGAGAT (SEQ ID NO: 9).

It is also specifically contemplated that one or more of SEQ ID Nos: 7, 8, or 9 can be used in a single shmiR, for example, a shmiR can comprise SEQ ID NO. 7 and 8, SEQ ID NO: 7 and 9, SEQ ID NO: 8 and 9, or SEQ ID Nos: 7, 8, and 9, or any other combination thereof.

It is specifically contemplated that different targeting and passenger strands specific for a given gene can be inserted into the above-recited miRNA frameworks to form a shmiR against a desired target gene. However, it is noted that when tandem shmiRs (e.g., 2, 3, or more) are part of a single construct, the miRNA frameworks for each shmiR should be different such that homologous recombination does not occur, thereby preventing proper function of each shmiR.

In one embodiment, the first BCL11A sequence comprises the sequence of: GCGCGATCGAGTGTTGAATAA (SEQ ID No: 4) and the second BCL11A sequence comprises the sequence of: TTATTCAACACTCGATCGCGC (SEQ ID NO: 5), wherein the first and second BCL11A sequences are complementary.

In one embodiment, the BCL11A sequence in an miR223 framework comprises the sequence of:

(SEQ ID NO: 6)
GATCTCACTTCCCCACAGAAGCTCTTGGCCTGGCCTCCTGCAGTGCCACGCTGCGCGATCGA

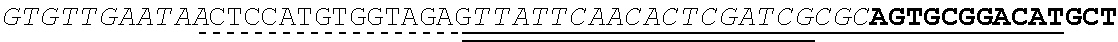

TACCAGCTCTAGGCCAGGGCAGATGGGATATGACGAATGGACTGCCAGCTGGATACAAGGAT
GCTCACC.
bold, underlined text: miR223 backbone
italicized text: BCL11A passenger strand sequence

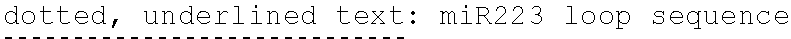

italics, double underlined text: BCL11A guide strand sequence

In one embodiment, the first ZNF410 sequence comprises the sequence of: GCTGAGCACTTAGTGTTTGTA (SEQ ID No: 10) and the second ZNF410 sequence comprises the sequence of: TACAAACACTAAGTGCTCAGC (SEQ ID NO: 11), wherein the first and second ZNF410 sequence are complementary.

In one embodiment, the ZNF410 sequence in an miR144 framework comprises the sequence of:

(SEQ ID NO: 12)
CGCTTTTCAAGCCATGCTTCCTGTGCCCCCAGTGGGGCCCTGGCTGGCTGAGCACTTAGTGT

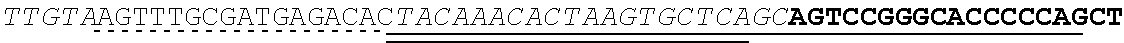

CTGGAGCCTGACAAGGAGGACAGGAGAGAT.
bold, underlined text: miR144 backbone
italicized text: ZNF410 passenger strand sequence

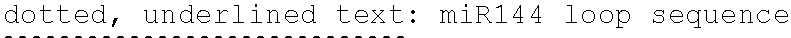

italics, double underlined text: ZNF410 guide strand sequence

In one embodiment, the first ZBTB7A sequence comprises the sequence of: ACGGGTACTTTTCATTCGCGC (SEQ ID No: 13) and the second ZBTB7A sequence comprises the sequence of: GCGCGAATGAAAAGTACCCGT (SEQ ID NO: 14), wherein the first and second ZBTB7A sequence are complementary.

In one embodiment, the ZBTB7A sequence in an miR144 framework comprises the sequence of:

(SEQ ID NO: 15)
CGCTTTTCAAGCCATGCTTCCTGTGCCCCCAGTGGGGCCCTGGCTGACGGGTACTTTTCATT

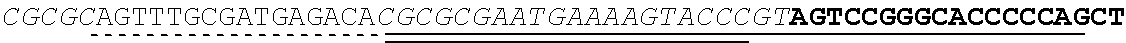

CTGGAGCCTGACAAGGAGGACAGGAGAGAT.
bold, underlined text: miR144 backbone
italicized text: ZBTB7A passenger strand sequence

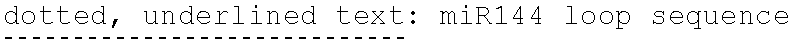

italics, double underlined text: ZBTB7A guide strand sequence

In one embodiment, the shmiR that targets BCL11A comprises the sequence of SEQ ID NO: 16.

(SEQ ID NO: 16)
CGCTTTTCAAGCCATGCTTCCTGTGCCCCCAGTGGGGCCCTGGC

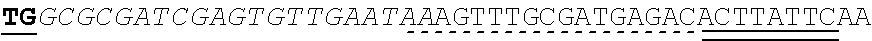

CACTCGATCGCGCAGTCCGGGCACCCCCAGCTCTGGAGCCTGACA
AGGAGGACAGGAGAGAT

Within the sequence of SEQ ID NO: 16, the bold, underlined text is an miRNA 144 backbone sequence, the italics text is a passenger strand sequence, the [symbol] text is a miRNA 144 loop sequence (SEQ ID NO: 8), and the double underlined text is a guide strand sequence.

### RNA Interference

The use of short hairpin RNAs within miRNA frameworks (shmiRs) can be used to inhibit the expression of selected target polypeptides to induce expression of fetal hemoglobin isoforms, such as BCL11A, ZNF410 and/or ZBTB7A, by mediating RNA interference. “RNA interference (RNAi)” is an evolutionarily conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. RNA interference is mediated herein using a construct that comprises tandem short hairpin RNAs that are embedded within an miRNA framework (i.e., a shmiR).

As used herein, the term “shRNA molecule” refers to a conventional stem-loop shRNA, which forms a precursor miRNA (pre-miRNA). When transcribed, a conventional shRNA forms a primary miRNA (pri-miRNA) or a structure very similar to a natural pri-miRNA. The pri-miRNA is subsequently processed by Drosha and its cofactors into pre-miRNA. In one embodiment, the shRNA portion of the shmiR are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. The tandem shmiRs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety). The target gene or sequence of the RNA interfering agent can be any cellular gene or genomic sequence, the inhibition of which can induce expression of fetal hemoglobin, e.g., the BCL11A sequence. A shmiR comprises a region that is substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target.

As used herein, “inhibition of target gene expression” includes any decrease in expression or protein activity or level of the target gene or protein encoded by the target gene (e.g., BCL11A, ZNF410 and/or ZBTB7A) as compared to a situation wherein no RNA interference has been induced (i.e., wherein the tandem shmiR construct is not introduced). The decrease is at least 10%, 20%, 30%, 40%, 50%, 60%, 70%, 80%, 90%, 95% or 99% or more as compared to the expression of a target gene or the activity or level of the protein encoded by a target gene which has not been targeted by one or more shmiRs from a tandem shmiR construct.

To ensure maximum safety and efficacy, each of the tandem shmiRs can be screened for potential off-target effects by, for example, expression profiling. Such methods are known to one skilled in the art and are described, for example, in Jackson et al. Nature Biotechnology 6:635-637, 2003. In addition to expression profiling, one can also screen the potential target sequences for similar sequences in the sequence databases to identify potential sequences which may have off-target effects. The shmiR sequences are chosen to maximize the uptake of the antisense (guide) strand of the shmiR or shRNA thereof into RISC and thereby maximize the ability of RISC to target BCL11A, ZNF410 and/or ZBTB7A mRNA for degradation. This can be accomplished by scanning for sequences that have the lowest free energy of binding at the 5′-terminus of the antisense strand. The lower free energy leads to an enhancement of the unwinding of the 5′-end of the antisense strand of the shRNA duplex, thereby ensuring that the antisense strand will be taken up by RISC and direct the sequence-specific cleavage of the human BCL11A, ZNF410 and/or ZBTB7A mRNA.

In some embodiments, the transcription of the shmiR can be driven by a polymerase (pol) III promoter. This class of promoters allows for utilization of appropriate regulatory elements for lineage or even cell-type specific expression. Alternatively, transcription of the shmiR can be driven by a pol II promoter.

In one embodiment, the construct comprising tandem shmiRs is delivered or administered in a pharmaceutically acceptable carrier. Additional carrier agents, such as liposomes, can be added to the pharmaceutically acceptable carrier. In another embodiment, the tandem shmiR construct comprises a vector encoding at least two tandem shmiRs in a pharmaceutically acceptable carrier. The shmiR or shRNA portion thereof is converted by the cells after transcription into siRNA capable of targeting, for example, BCL11A.

In one embodiment, the vector or expression construct comprises an erythroid promoter such as the bovine growth hormone (BGH) promoter or a modified promoter thereof.

In one embodiment, the vector is a regulatable vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif) can be used.

In one embodiment, the shmiR constructs used in the methods described herein are administered to and are taken up actively by cells in vivo following intravenous injection, e.g., hydrodynamic injection, without the use of a vector or in a naked plasmid form. Other delivery methods include delivery of the shmiR construct using a basic peptide by conjugating or mixing the construct with a basic peptide, e.g., a fragment of a TAT peptide, mixing with cationic lipids or formulating into particles. The shmiR construct can be delivered singly, or in combination with other RNA interference agents, e.g., additional miRNAs or siRNAs directed to other cellular genes. The shmiR construct can also be administered in combination with other pharmaceutical agents which are used to treat or prevent hemoglobinopathies.

Synthetic nucleic acid molecules, including the tandem shmiR construct, can be obtained using a number of techniques known to those of skill in the art. For example, the molecule can be chemically synthesized or produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197).

The targeted region of the shRNA molecule of the shmiR construct can be selected from a given target gene sequence, e.g., a BCL11A coding sequence, beginning from about 25 to 50 nucleotides, from about 50 to 75 nucleotides, or from about 75 to 100 nucleotides downstream of the start codon. Nucleotide sequences can contain 5′ or 3′ UTRs and regions nearby the start codon. One method of designing a shmiR or shRNA thereof involves scanning for a 23 nucleotide sequence motif and selecting hits with at least 25%, 30%, 35%, 40%, 45%, 50%, 55%, 60%, 65%, 70% or 75% G/C content. Alternatively, if no such sequence is found, the search can be extended using the motif NA(N21), where N can be any nucleotide. Analysis of sequence databases, including but not limited to the NCBI, BLAST, Derwent, and GenSeq as well as commercially available oligosynthesis companies such as OLIGOENGINE®, can also be used to select shRNA sequences against EST libraries to ensure that only one gene is targeted.

Lentiviral vectors that can be used to encode tandem shmiRs include, but are not limited to, vectors derived from human immunodeficiency virus (e.g., HIV-1, HIV-2), feline immunodeficiency virus (FIV), simian immunodeficiency virus (SIV), bovine immunodeficiency virus (BIV), equine infectious anemia virus (EIAV), and alpha retrovirus. These vectors can be constructed and engineered using art-recognized techniques to increase their safety for use in therapy and to include suitable expression elements and therapeutic genes, which encode shmiRs or shRNAs thereof for treating conditions including, but not limited to, hemoglobinopathies.

In consideration of the potential toxicity of lentiviruses, the vectors can be designed in different ways to increase their safety in gene therapy applications. For example, the vector can be made safer by separating the necessary lentiviral genes (e.g., gag and pol) onto separate vectors as described, for example, in U.S. Pat. No. 6,365,150, the contents of which are incorporated by reference herein. Thus, recombinant retrovirus can be constructed such that the retroviral coding sequence (gag, pol, env) is replaced by a gene of interest rendering the retrovirus replication defective. The replication defective retrovirus is then packaged into virions through the use of a helper virus or a packaging cell line, by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals.

In one embodiment, packaging cell lines are used to propagate vectors (e.g., lentiviral vectors) to increase the titer of the vector virus. The use of packaging cell lines is also considered a safe way to propagate the virus, as use of the system reduces the likelihood that recombination will occur to generate wild-type virus. In addition, to reduce toxicity to cells caused by expression of packaging proteins, packaging systems can be used in which the plasmids encoding the packaging functions of the virus are only transiently transfected by, for example, chemical means.

In another embodiment, the vector can be made safer by replacing certain lentiviral sequences with non-lentiviral sequences. Thus, lentiviral vectors can contain partial (e.g., split) gene lentiviral sequences and/or non-lentiviral sequences (e.g., sequences from other retroviruses) as long as its function (e.g., viral titer, infectivity, integration and ability to confer high levels and duration of therapeutic gene expression) are not substantially reduced. Elements which may be cloned into the viral vector include, but are not limited to, promoter, packaging signal, LTR(s), polypurine tracts, and a reverse response element (RRE). In one embodiment, the LTR region is modified by replacing the viral LTR promoter with a heterologous promoter. In one embodiment, the promoter of the 5′ LTR is replaced with a heterologous promoter. Examples of heterologous promoters which can be used include, but are not limited to, a spleen focus-forming virus (SFFV) promoter, a tetracycline-inducible (TET) promoter, a β-globin locus control region and a β-globin promoter (LCR), and a cytomegalovirus (CMV) promoter.

The promoter of the lentiviral vector can be one which is naturally (i.e., as it occurs with a cell in vivo) or non-naturally associated with the 5′ flanking region of a particular gene. Promoters can be derived from eukaryotic genomes, viral genomes, or synthetic sequences. Promoters can be selected to be non-specific (active in all tissues) (e.g., SFFV), tissue specific (e.g., (LCR), regulated by natural regulatory processes, regulated by exogenously applied drugs (e.g., TET), or regulated by specific physiological states such as those promoters which are activated during an acute phase response or those which are activated only in replicating cells. Non-limiting examples of promoters that can be used with a tandem shmiR construct include the spleen focus-forming virus promoter, a tetracycline-inducible promoter, a β-globin locus control region and a β-globin promoter (LCR), a cytomegalovirus (CMV) promoter, retroviral LTR promoter, cytomegalovirus immediate early promoter, SV40 promoter, and dihydrofolate reductase promoter. The promoter can also be selected from those shown to specifically express in the select cell types which can be found associated with conditions including, but not limited to, hemoglobinopathies. In one embodiment, the promoter is cell specific such that gene expression is restricted to red blood cells. Erythrocyte-specific expression is achieved by using the human β-globin promoter region and locus control region (LCR).

Skilled practitioners will recognize that selection of the promoter to express the polynucleotide of interest will depend on the vector, the nucleic acid cassette, the cell type to be targeted, and the desired biological effect. Skilled practitioners will also recognize that in the selection of a promoter, the parameters can include: achieving sufficiently high levels of gene expression to achieve a physiological effect; maintaining a critical level of gene expression; achieving temporal regulation of gene expression; achieving cell type specific expression; achieving pharmacological, endocrine, paracrine, or autocrine regulation of gene expression; and preventing inappropriate or undesirable levels of expression. Any given set of selection requirements will depend on the conditions but can be readily determined once the specific requirements are determined. In one embodiment, the promoter is cell-specific such that gene expression is restricted to red blood cells. As a non-limiting example, erythrocyte-specific expression is achieved by using the human β-globin promoter region and locus control region (LCR).

Standard techniques for the construction of expression vectors suitable for use with tandem shmiRs are well-known to those of ordinary skill in the art and can be found in such publications as Sambrook et al. (1989) Molecular Cloning: A Laboratory Manual, 2nd Ed. Cold Spring Harbor, N.Y. A variety of strategies are available for ligating fragments of DNA, the choice of which depends on the nature of the termini of the DNA fragments and which choices can be readily made by the skilled artisan.

Gene therapy vectors encoding shmiRs can be used to express a variety of therapeutic shRNAs in transformed erythroid cells. In one embodiment, one or more shmiRs encoded and expressed by the vector is derived from a gene that can be used to treat a hemoglobinopathy, such as BCL11A, ZNF410 and/or ZBTB7A.

Retroviral vectors, including lentiviral vectors, as described above or cells comprising the same, can be administered in vivo to subjects by any suitable route, as is well known in the art. The term “administration” refers to the route of introduction of a formulated vector into the body. For example, administration may be intravascular, intraarterial, intravenous, intramuscular, topical, oral, or by gene gun or hypospray instrumentation. Thus, administration can be direct to a target tissue or through systemic delivery. Administration can be direct injection into the bone marrow. Administration directly to the target tissue can involve needle injection, hypospray, electroporation, or the gene gun. See, e.g., WO 93/18759, which is incorporated by reference herein.

Alternatively, nucleic acid vectors encoding shmiRs can be administered ex vivo or in vitro to cells or tissues using standard transfection techniques well known in the art.

In one embodiment, the retroviral vectors for use in encoding tandem shmiRs can also be transduced into host cells, including embryonic stem cells, somatic stem cells, or progenitor cells. Examples of progenitor host cells which can be transduced by the retroviral vectors include precursors of erythrocytes and hematopoietic stem cells. In another embodiment, the host cell is an erythrocyte. Transduced host cells can be used as a method of achieving erythroid-specific expression of the gene of interest in the treatment of hemoglobinopathies.

In another embodiment of this aspect and all other aspects provided herein, the first segment of the miR223 miRNA comprises the sequence of

(SEQ ID NO: 1)
GATCTCACTTCCCCACAGAAGCTCTTGGCCTGGCCTCCTGCAGTGCCAC
GCT.

In another embodiment of this aspect and all other aspects provided herein, the loop segment comprises a segment of miR223. In one embodiment of this aspect and all other aspects provided herein, the miR223 loop segment comprises the sequence of:

(SEQ ID NO: 2)
CTCCATGTGGTAGAG.

In another embodiment of this aspect and all other aspects provided herein, the second segment of the miR223 miRNA comprises the sequence of:

(SEQ ID NO: 3)
AGTGCGGCACATGCTTACCAGCTCTAGGCCAGGGCAGATGGGATA
TGACGAATGGACTGCCAGCTGGATACAAGGATGCTCACC.

In another embodiment of this aspect and all other aspects provided herein, the first BCL11A sequence comprises the sequence of: GCGCGATCGAGTGTTGAATAA (SEQ ID No: 4) and the second BCL11A sequence comprises the sequence of: TTATTCAACACTCGATCGCGC (SEQ ID NO: 5), wherein the first and second BCL11A sequence are complementary.

In another embodiment of this aspect and all other aspects provided herein, the BCL11 A sequence in an miR223 framework comprises the sequence of:

(SEQ ID NO: 6)
GATCTCACTTCCCCACAGAAGCTCTTGGCCTGGCCTCCTGCAGTGCCACGCTGCGCGATCGA

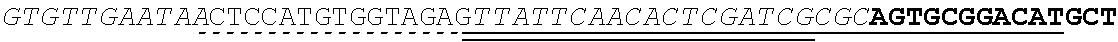

TACCAGCTCTAGGCCAGGGCAGATGGGATATGACGAATGGACTGCCAGCTGGATACAAGGAT
GCTCACC.
bold, underlined text: miR223 backbone
italicized text: BCL11A passenger strand sequence

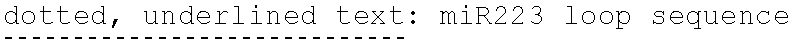

italics, double underlined text: BCL11A guide strand sequence

In another embodiment of this aspect and all other aspects provided herein, the first segment of the miR144 miRNA comprises the sequence of:

(SEQ ID NO: 7)
CGCTTTTCAAGCCATGCTTCCTGTGCCCCCAGTGGGGCCCTGGCTG.

In another embodiment of this aspect and all other aspects provided herein, the loop segment comprises a segment of miR144. In one embodiment of this aspect and all other aspects provided herein, the miR144 loop segment comprises the sequence of AGTTTGCGATGAGACAC (SEQ ID NO: 8).

In another embodiment of this aspect and all other aspects provided herein, the second segment of the miR144 miRNA comprises the sequence of:

(SEQ ID NO: 9)
AGTCCGGGCACCCCCAGCTCTGGAGCCTGACAAGGAGGACAGGAGAGAT.

In another embodiment of this aspect and all other aspects provided herein, the first ZNF410 sequence comprises the sequence of: GCTGAGCACTTAGTGTTTGTA (SEQ ID No: 10) and the second ZNF410 sequence comprises the sequence of: TACAAACACTAAGTGCTCAGC (SEQ ID NO: 11), wherein the first and second ZNF410 sequence are complementary.

In another embodiment of this aspect and all other aspects provided herein, the ZNF410 sequence in an miR144 framework comprises the sequence of:

(SEQ ID NO: 12)
CGCTTTTCAAGCCATGCTTCCTGTGCCCCCAGTGGGGCCCTGGCTGGCTGAGCACTTAGTGT

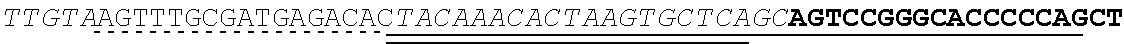

CTGGAGCCTGACAAGGAGGACAGGAGAGAT.
bold, underlined text: miR144 backbone
italicized text: ZNF410 passenger strand sequence

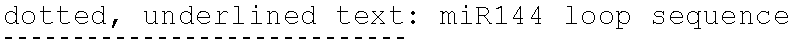

italics, double underlined text: ZNF410 guide strand sequence

In another embodiment of this aspect and all other aspects provided herein, the first ZBTB7A sequence comprises the sequence of: ACGGGTACTTTTCATTCGCGC (SEQ ID No: 13) and the second ZBTB7A sequence comprises the sequence of: GCGCGAATGAAAAGTACCCGT (SEQ ID NO: 14), wherein the first and second ZBTB7A sequence are complementary.

In another embodiment of this aspect and all other aspects provided herein, the ZBTB7A sequence in an miR144 framework comprises the sequence of:

(SEQ ID NO: 15)
CGCTTTTCAAGCCATGCTTCCTGTGCCCCCAGTGGGGCCCTGGCTGACGGGTACTTTTCATT

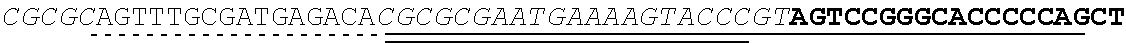

CTGGAGCCTGACAAGGAGGACAGGAGAGAT.
bold, underlined text: miR144 backbone
italicized text: ZBTB7A passenger strand sequence

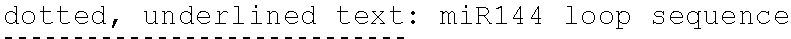

italics, double underlined text: ZBTB7A guide strand sequence

In another embodiment of this aspect and all other aspects provided herein, the shmiR that targets BCL11A comprises the sequence of SE ID NO: 16.

(SEQ ID NO: 16)
CGCTTTTCAAGCCATGCTTCCTGTGCCCCCAGTGGGGCCCTGGC

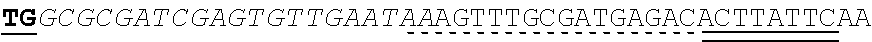

CACTCGATCGCGCAGTCCGGGCACCCCCAGCTCTGGAGCCTGACA
AGGAGGACAGGAGAGAT

Within the sequence of SEQ ID NO: 16, the bold, underlined text is an miRNA 144 backbone sequence, the italics text is a passenger strand sequence, the [symbol] text is a miRNA 144 loop sequence (SEQ ID NO: 8), and the double underlined text is a guide strand sequence.

In another embodiment of this aspect and all other aspects provided herein, the first and second BCL11a, ZNF410 or ZBTB7A segments are complementary.

In another embodiment of this aspect and all other aspects provided herein, the first shmiR and the at least second shmiR do not undergo homologous recombination when introduced into a cell.

In other embodiments, the BCL11A shmiR or shRNA thereof comprises the following sequences:

BCL11A miR1 oligos:
Sense
(SEQ ID NO: 17)
ACGCTCGCACAGAACACTCATGGATTctccatgtggtagagAATCC
ATGAGTGTTCTGTGCGAG
Anti-sense
(SEQ ID NO: 18)
CGCACTCGCACAGAACACTCATGGATTctctaccacatggagAATC
CATGAGTGTTCTGTGCGA
BCL11A miR2 oligos:
Sense
(SEQ ID NO: 19)
ACGCTCCAGAGGATGACGATTGTTTActccatgtggtagagTAAAC
AATCGTCATCCTCTGGag
Anti-sense
(SEQ ID NO: 20)
CGCActCCAGAGGATGACGATTGTTTActctaccacatggagTAAA
CAATCGTCATCCTCTGGa
BCL11A D12G5-2 shRNA:
Sense
(SEQ. ID. NO: 21)
ACGCTCGCACAGAACACTCATGGATTctccatgtggtagagAATCC
ATGAGTGTTCTGTGCGAG
Anti-sense
(SEQ. ID. NO: 22)
CGCACTCGCACAGAACACTCATGGATTctctaccacatggagAATC
CATGAGTGTTCTGTGCGA

### Pharmaceutical Compositions

The nucleic acids encoding tandem shmiRs as described herein can be formulated as a pharmaceutical composition (e.g., include at least one pharmaceutically acceptable carriers). In one embodiment, the composition includes a tandem shmiR construct (e.g., plasmid or vector encoding tandem shmiRs) in a therapeutically effective amount sufficient to treat or reduce the risk of developing (e.g. ameliorate the symptoms of) a hemoglobinopathy and a pharmaceutically acceptable carrier.

As used herein, the term “pharmaceutically acceptable,” and grammatical variations thereof, as they refer to compositions, carriers, diluents and reagents, are used interchangeably and represent that the materials are capable of administration to or upon a subject without the production of undesirable physiological effects such as nausea, dizziness, gastric upset, and the like. Each carrier must also be “acceptable” in the sense of being compatible with the other ingredients of the formulation. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. The pharmaceutical formulation comprises a tandem shmiR construct in combination with one or more pharmaceutically acceptable ingredients. The carrier can be in the form of a solid, semi-solid or liquid diluent, cream or a capsule. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic, and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. The phrase “pharmaceutically acceptable carrier or diluent” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body.

Sterile injectable solutions can be prepared by incorporating a tandem shmiR construct in a therapeutically effective amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Administration and Efficacy

As disclosed herein, provided herein are methods for increasing fetal hemoglobin levels in a subject. Accordingly, one aspect provides a method for increasing fetal hemoglobin levels in a subject in need thereof, the method comprising the step of contacting a hematopoietic progenitor cell or a HSC in vivo, in vitro or ex vivo with an effective amount of a tandem shmiR construct, optionally administering treated cells to a subject when treated in vitro or ex vivo, whereby HbF expression is increased, relative to expression prior to such contacting. In one embodiment, the tandem shmiR construct comprises an inhibitor of BCL11A (e.g., a shmiR or shRNA thereof that acts as an RNA interference agent).

Accordingly, in one embodiment, the subject has been diagnosed with a hemoglobinopathy. In a further embodiment, the hemoglobinopathy is a SCD. As used herein, SCD can be sickle cell anemia, sickle-hemoglobin C disease (HbSC), sickle beta-plus-thalassaemia (HbS/β+), or sickle beta-zero-thalassaemia (HbS/β0). In another preferred embodiment, the hemoglobinopathy is THAL.

In one embodiment, the methods and compositions described herein can reduce or ameliorate one or more symptoms associated with the disorder by increasing the amount of fetal hemoglobin in the individual. Symptoms typically associated with a hemoglobinopathy, include for example, anemia, tissue hypoxia, organ dysfunction, abnormal hematocrit values, ineffective erythropoiesis, abnormal reticulocyte (erythrocyte) count, abnormal iron load, the presence of ring sideroblasts, splenomegaly, hepatomegaly, impaired peripheral blood flow, dyspnea, increased hemolysis, jaundice, anemic pain crises, acute chest syndrome, splenic sequestration, priapism, stroke, hand-foot syndrome, and pain such as angina pectoris.

In one embodiment, the hematopoietic progenitor cell or HSC is contacted ex vivo or in vitro with a tandem shmiR construct, and the cell or its progeny is administered to the subject. In a further embodiment, the hematopoietic progenitor cell is a cell of the erythroid lineage.

In one embodiment, the hematopoietic progenitor cell or HSC is contacted with a composition comprising a tandem shmiR construct and a pharmaceutically acceptable carrier or diluent. In one embodiment, the composition is administered by injection, infusion, instillation, or ingestion. In one embodiment, the composition is administered by direct injection into the bone marrow.

In one embodiment of any one method described, the methods and compositions described herein are used to treat, prevent, or ameliorate a hemoglobinopathy selected from the group consisting of: hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease.

In various embodiments of any one method described, the tandem shmiRs are administered by direct injection to a cell, tissue, or organ of a subject in need of gene therapy, in vivo. In various other embodiments of any one method described, cells are transduced in vitro or ex vivo with tandem shmiR constructs (or vectors thereof), and optionally expanded ex vivo. The transduced cells are then administered to a subject in need of gene therapy.

In one embodiment of any one method described, the method further comprises selecting a subject in need of the gene therapy described. For example, a subject exhibiting symptoms or cytology of a hemoglobinopathy is selected from the group consisting of hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease. Alternatively, the subject carries a genetic mutation that is associated with a hemoglobinopathy, a genetic mutation described herein. For example, a subject diagnosis of SCD with genotype HbSS, HbS/(30 thalassemia, HbSD, or HbSO, and/or with HbF<10% by electrophoresis.

It is to be noted that dosage values can vary with the severity of the condition to be alleviated. It is to be further understood that for any particular subject, specific dosage regimens can be adjusted over time according to the individual need and the professional judgment of the person administering or supervising the administration of the compositions, and that dosage ranges set forth herein are exemplary only and are not intended to limit the scope or practice of the claimed composition. In one embodiment, the dosage ranges from 10³-10¹⁴ viral particles/50 kg weight. In other embodiments, the dosage ranges from 10³-10¹³ viral particles/50 kg weight, 10³-10¹² viral particles/50 kg weight, 10³-10¹¹ viral particles/50 kg weight, 10³-10¹⁰ viral particles/50 kg weight, 10³-10⁹ viral particles/50 kg weight, 10³-10⁸ viral particles/50 kg weight, 10³-10⁷ viral particles/50 kg weight, 10³-10⁶ viral particles/50 kg weight, 10³-10⁵ viral particles/50 kg weight, 10⁴-10¹³ viral particles/50 kg weight, 10⁵-10¹³ viral particles/50 kg weight, 10⁶-10¹³ viral particles/50 kg weight, 10⁷-10¹³ viral particles/50 kg weight, 10⁸-10¹³ viral particles/50 kg weight, 10⁹-10¹³ viral particles/50 kg weight, 10¹⁰-10¹³ viral particles/50 kg weight, 10¹¹-10¹³ viral particles/50 kg weight, 10¹²-10¹³ viral particles/50 kg weight, 10⁵-10¹⁰ viral particles/50 kg weight, 10³-10⁸ viral particles/50 kg weight 10⁶-10⁹ viral particles/50 kg weight, or any integer therebetween.

The amount of a tandem shmiR construct in the composition can vary according to factors such as the disease state, age, sex, and weight of the individual. Dosage regimens can be adjusted to provide the optimum therapeutic response. For example, a single bolus can be administered, several divided doses may be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals. However, for any given case, an appropriate “effective amount” can be determined by one of ordinary skill in the art using only routine experimentation.

In some embodiments, cells can be genetically modified to express the therapeutic tandem shmiR molecules, for use in the treatment of hemoglobinopathies. As used herein, the term “genetically engineered” or “genetically modified” refers to the addition, deletion, or modification of the genetic material in a cell. The terms, “genetically modified cells,” “modified cells,” and, “redirected cells,” are used interchangeably. In particular embodiments, cells transduced with vectors contemplated herein are genetically modified.

In various embodiments, the genetically modified cells contemplated herein are transduced in vitro or ex vivo with a construct encoding tandem shmiRs, and optionally expanded ex vivo. The transduced cells are then administered to a subject in need of gene therapy.

Cells suitable for transduction and administration in the gene therapy methods contemplated herein include, but are not limited to stem cells, progenitor cells, and differentiated cells. In certain embodiments, the transduced cells are embryonic stem cells, bone marrow stem cells, umbilical cord stem cells, placental stem cells, mesenchymal stem cells, hematopoietic stem cells, erythroid progenitor cells, and erythroid cells. In one embodiment, the transduced cell is a hematopoietic stem cell or differentiated progeny thereof.

Engineered cells for treatment of hemoglobinopathies can be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells expressing tandem shmiRs are allogeneic. An “isolated cell” refers to a cell that has been obtained from an in vivo tissue or organ and is substantially free of extracellular matrix.

Illustrative examples of genetically modified cells suitable for cell-based therapies contemplated herein include, but are not limited to: embryonic stem cells, bone marrow stem cells, umbilical cord stem cells, placental stem cells, mesenchymal stem cells, hematopoietic stem cells, hematopoietic progenitor cells, myeloid progenitors, erythroid progenitors, and other erythroid cells.

In some embodiments, cells suitable for cell-based therapies contemplated herein include, but are not limited to: hematopoietic stem or progenitor cells, proerythroblasts, basophilic erythroblasts, polychromatic erythroblasts, orthochromatic erythroblasts, polychromatic erythrocytes, and erythrocytes (RBCs), or any combination thereof.

In a particular embodiment, a method of preventing, ameliorating, or treating a hemoglobinopathy in a subject is provided. The method comprises administering a population of cells comprising hematopoietic cells transduced with a tandem shmiR construct.

In particular embodiments, a population of cells administered to a subject comprises hematopoietic stem or progenitor cells, proerythroblasts, basophilic erythroblasts, polychromatic erythroblasts, orthochromatic erythroblasts, polychromatic erythrocytes, and erythrocytes (RBCs), or any combination thereof, and any proportion of which may be genetically modified using a tandem shmiR construct is contemplated herein.

The genetically modified cells may be administered as part of a bone marrow or cord blood transplant in an individual that has or has not undergone bone marrow ablative therapy. In one embodiment, genetically modified cells contemplated herein are administered in a bone marrow transplant to an individual that has undergone chemoablative or radioablative bone marrow therapy.

In one embodiment, a dose of genetically modified cells is delivered to a subject intravenously. In one embodiment, genetically modified hematopoietic cells are intravenously administered to a subject.

In particular embodiments, patients receive a dose of genetically modified cells, e.g., hematopoietic stem cells, of about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single intravenous dose. In certain embodiments, patients receive a dose of genetically modified cells, e.g., hematopoietic stem cells, of at least 1×10⁵ cells/kg, at least 5×10⁵ cells/kg, at least 1×10⁶ cells/kg, at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, at least 1×10⁷ cells/kg, at least 5×10⁷ cells/kg, at least 1×10⁸ cells/kg, or more in one single intravenous dose.

In an additional embodiment, patients receive a dose of genetically modified cells, e.g., hematopoietic stem cells, of about 1×10⁵ cells/kg to about 1×10⁸ cells/kg, about 1×10⁶ cells/kg to about 1×10⁸ cells/kg, about 1×10⁶ cells/kg to about 9×10⁶ cells/kg, about 2×10⁶ cells/kg to about 8×10⁶ cells/kg, about 2×10⁶ cells/kg to about 8×10⁶ cells/kg, about 2×10⁶ cells/kg to about 5×10⁶ cells/kg, about 3×10⁶ cells/kg to about 5×10⁶ cells/kg, about 3×10⁶ cells/kg to about 4×10⁸ cells/kg, or any intervening dose of cells/kg.

In various embodiments, the methods comprise administering a population or dose of cells comprising about 5% transduced cells, about 10% transduced cells, about 15% transduced cells, about 20% transduced cells, about 25% transduced cells, about 30% transduced cells, about 35% transduced cells, about 40% transduced cells, about 45% transduced cells, or about 50% transduced cells, to a subject.

In one embodiment of any methods described, as used herein, “administered” refers to the placement of a tandem shmiR construct into a subject by a method or route which results in at least partial localization of the tandem shmiRs at a desired site. The tandem shmiR construct can be administered by any appropriate route which results in effective treatment in the subject, i.e., administration results in delivery to a desired location in the subject where at least a portion of the composition delivered, i.e., at least one shmiR or shRNA thereof, which inhibits e.g., BCL11A, is active in the desired site for a period of time. The period of time the inhibitor is active depends on the half-life in vivo after administration to a subject, and can be as short as a few hours, e.g., at least 1 hour, at least 2 hours, at least 3 hours, at least 4 hours, at least 5 hours, at least 6 hours, at least 8 hours, at least 10 hours, at least 12 hours, at least 18 hours, at least 24 hours, to a few days. Modes of administration include injection, infusion, instillation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

In one embodiment, the composition described herein, or the virus or vector carrying a nucleic acid molecule encoding tandem shmiRs is injected into the bone marrow.

In one embodiment, the hematopoietic progenitor cell or HSC from a subject needing treatment is contacted with a composition comprising a tandem shmiR construct as described herein. In other embodiments, the composition comprises a virus or vector carrying a tandem shmiR construct as described herein. The subject needing treatment is one diagnosed with a hemoglobinopathy such as SCD or THAL.

As an example of a method of treatment of a subject or reducing the risk of developing a hemoglobinopathy in a subject, the method comprises administering to the subject a composition comprising either a tandem shmiR construct or modified engineered cells treated with a tandem shmiR construct. In one embodiment, the method further comprises identifying a subject having a hemoglobinopathy or is at risk of developing a hemoglobinopathy. In another embodiment, the method further comprises selecting the identified subject having a hemoglobinopathy or is at risk of developing a hemoglobinopathy.

As another example of a method of treatment of a subject or reducing the risk of developing a hemoglobinopathy in a subject, the method comprises the following steps: mobilize the hematopoietic stem and hematopoietic progenitor cells in a subject; harvest and collect peripheral blood from the subject, positive selection of CD34+ cells from the peripheral blood, transduce or transfect the CD34+ selected cells in vitro with a tandem shmiR construct as described herein; wash the transduced CD34+ selected cells; and administer the cells into the subject. In one embodiment, the method further comprises identifying a subject having a hemoglobinopathy or at risk of developing a hemoglobinopathy. In one embodiment, the method further comprises selecting the subject having a hemoglobinopathy or at risk of developing a hemoglobinopathy. In another embodiment, the method further comprises expanding in culture the washed, transduced CD34+ selected cells in vitro prior to administering to the subject. In another embodiment, the method further comprises differentiating in culture the washed, transduced CD34+ selected cells in vitro prior to administering to the subject.

As another example of a method of treatment of a subject or reducing the risk of developing a hemoglobinopathy in a subject, the method comprises the following steps: mobilize the hematopoietic stem and hematopoietic progenitor cells in a donor subject; harvest and collect peripheral blood from the donor subject, positive selection of CD34+ cells from the peripheral blood, transduce or transfect the CD34+ selected cells in vitro with a tandem shmiR construct as described herein; wash the transduced CD34+ selected cells; and administer the cells into a recipient subject. In one embodiment, the method further comprises selecting a recipient subject having a hemoglobinopathy or is at risk of developing a hemoglobinopathy. In another embodiment, the method further comprises expanding in culture the washed, transduced CD34+ selected cells in vitro prior to administering to the recipient subject. In another embodiment, the method further comprises differentiating in culture the washed, transduced CD34+ selected cells in vitro prior to administering to the recipient subject.

As another example of a method of treatment of a subject or reducing the risk of developing a hemoglobinopathy in a subject, the method comprises the following steps: harvest and collect the blood from the bone marrow of a subject, positive selection of CD34+ cells from the bone marrow blood, transduce or transfect the CD34+ selected cells in vitro with a tandem shmiR construct as described herein; wash the transduced CD34+ selected cells; and administer the cells into the subject. In one embodiment, the method further comprises identifying a subject having a hemoglobinopathy or is at risk of developing a hemoglobinopathy. In one embodiment, the method further comprises selecting the subject having a hemoglobinopathy or is at risk of developing a hemoglobinopathy. In another embodiment, the method further comprises expanding in culture the washed, transduced CD34+ selected cells in vitro prior to administering to the subject. In another embodiment, the method further comprises differentiating in culture the washed, transduced CD34+ selected cells in vitro prior to administering to the subject.

As another example of a method of treatment of a subject or reducing the risk of developing a hemoglobinopathy in a subject, the method comprises the following steps: harvest and collect the blood from the bone marrow of a donor subject, positive selection of CD34+ cells from the bone marrow blood, transduce or transfect the CD34+ selected cells in vitro with a tandem shmiR construct as described herein; wash the transduced CD34+ selected cells; and administer the cells into a recipient subject. In one embodiment, the method further comprises identifying a recipient subject having a hemoglobinopathy or is at risk of developing a hemoglobinopathy. In one embodiment, the method further comprises selecting a recipient subject having a hemoglobinopathy or is at risk of developing a hemoglobinopathy. In another embodiment, the method further comprises expanding in culture the washed, transduced CD34+ selected cells in vitro prior to administering to the recipient subject. In another embodiment, the method further comprises differentiating in culture the washed, transduced CD34+ selected cells in vitro prior to administering to the recipient subject.

In one embodiment, the disclosure herein provides a modified engineered cell comprising a nucleic acid sequence encoding tandem shmiRs as described herein.

In one embodiment, the disclosure herein provides a modified engineered cell that has been transduced or transfected with a tandem shmiR construct as described herein. In one embodiment, the vector is a lentivirus.

In one embodiment, the disclosure herein provides a method of treatment of a subject or reducing the risk of developing a hemoglobinopathy in a subject, the method comprises administering a modified engineered cell that has been transduced or transfected with a tandem shmiR construct as described herein. In one embodiment, the vector is a lentivirus.

In one embodiment, the modified engineered cell is an embryonic stem cell, a somatic stem cell, a progenitor cell, a bone marrow cell, a hematopoietic stem cell, or a hematopoietic progenitor cell. In one embodiment, the modified engineered cell is a cell that has been differentiated from an embryonic stem cell, a somatic stem cell, a progenitor cell, a bone marrow cell, a hematopoietic stem cell, or a hematopoietic progenitor cell. In one embodiment, the modified engineered cell is a cell that has been differentiated into the erythroid lineage. In one embodiment, the modified engineered cell is a cell that has been differentiated into an erythrocyte. In one embodiment, the modified engineered cell is a CD34+ cell.

### Additional Embodiments Related to Methods of Treating, or Reducing Risk of Developing, a Hemoglobinopathy

Provided herein are improved compositions and methods for increasing fetal hemoglobin (HbF) production in a cell, by administering a tandem shmiR construct, for example, a construct that inhibits expression of BCL11A, ZNF410 or ZBTB7A. In some embodiments, the compositions and methods described herein can be used to increase fetal hemoglobin levels in a cell. In some embodiments, the cell is an embryonic stem cell, a somatic stem cell, a progenitor cell, a bone marrow cell, a hematopoietic stem cell, a hematopoietic progenitor cell or a progeny thereof.

Accordingly, one aspect described herein provides methods for increasing fetal hemoglobin levels expressed by a cell, comprising the steps of contacting an embryonic stem cell, a somatic stem cell, a progenitor cell, a bone marrow cell, a hematopoietic stem cell, or a hematopoietic with an effective amount of a composition comprising at least a virus or vector comprising a tandem shmiR construct as described herein, whereby the expression of e.g., BCL11A, ZNF410 and/or ZBTB7A is reduced and the fetal hemoglobin expression is increased in the cell, or its progeny, relative to the cell prior to such contacting. In one embodiment, the vector or virus expresses an RNA interference agent which is a BCL11A shmiR which inhibits BCL11A, thereby reducing the expression of BCL11A. In some embodiments, each of the shmiRs in the tandem shmiR construct targets BCL11A. In other embodiments, one the shmiRs in the tandem shmiR construct targets BCL11A while at least one other shmiR in the construct targets another gene the inhibition of which induces fetal hemoglobin (e.g., ZNF410 or ZBTB7A). In one embodiment, the tandem shmiR construct encodes a shmiR that targets BCL11A and a shmiR that targets ZNF410. In one embodiment, the tandem shmiR construct encodes a shmiR that targets BCL11A and a shmiR that targets ZBTB7A. In one embodiment, the tandem shmiR construct encodes a shmiR that targets BCL11A, a shmiR that targets ZNF410, and a shmiR that targets ZBTB7A.

In some embodiments of any of the methods described herein, the subject is suspected of having, is at risk of having, or has a hemoglobinopathy, e.g., SCD or THAL. It is well within the skills of an ordinary practitioner to recognize a subject that has, or is at risk of developing, a hemoglobinopathy.

The subjects can also be those undergoing any of a variety of additional therapy treatments. Thus, for example, subjects can be those being treated with oxygen, hydroxyurea, folic acid, or a blood transfusion.

The invention is further described in the following examples, which do not limit the scope of the invention described in the claims.

### EXAMPLES

β-hemoglobinopathies, including sickle-cell disease (SCD) and β-thalassemia (β-thal), are caused by mutations in the hemoglobin β subunit gene (HBB) that affect the production or structure of adult hemoglobin. Induction of HbF has been a long-term goal for the treatment of β-hemoglobinopathies, and continued production or reactivation of HbF in adults effectively reduces many of the most serious complications of the disease phenotype, especially in the SCD [1,2]. BCL11A is a promising target for the treatment of (β-hemoglobinopathy due to its critical role in HbF silencing.

Clinical trials aimed at downregulating BCL11A shows immense promise in elevating HbF and resolving disease complications. However, achieving the high-efficiency gene transfer and robust γ-globin production required to correct the SCD phenotype (>20%) and β-thal remains a challenge. More powerful and versatile vectors are required, to achieve therapeutic levels of HbF production in the most severe genotypes. The zinc-finger protein (ZNF)410 is a novel HbF repressor. Its chromatin occupancy is concentrated only at CHD4, which encodes the NuRD nucleosome remodeler; NuRD itself is required for HbF repression, and is independent of BCL11A. Here, the inventors combine BCL11A knockdown via a microRNA embedded shRNA (shmiR) and ZNF410 shmiR into a single erythroid-specific lentivirus vector to express two shmiR simultaneously, and show more efficient HbF induction than the current BCL11A single shmiR vector.

### Example 1: Development of a Double shmiR Lentivirus Effectively Targeting Both BCL11A and ZNF410 for Enhanced Induction of Fetal Hemoglobin to Treat β-Hemoglobinopathies

Beta (β)-hemoglobinopathies, including sickle-cell disease (SCD) and (β-thalassemia (β-thal), are inherited blood disorders that have serious health effects and shorten lifespans for millions of people around the world.¹⁻³ Both diseases are caused by mutations in the hemoglobin 3 gene (HBB) that affect the production or structure of adult hemoglobin. Mutant β-sickle globin (β^{S}) is associated with intracellular hemoglobin polymerization in the deoxygenated state leading to sickle-shaped and stiffened red blood cells (RBCs) that block capillaries and have a short circulating half-life.⁴⁻⁶ This leads to life-long morbidity, ischemic end organ damage and a shortened lifespan in many individuals. β-thal is caused by more than 200 different β-globin gene mutations that reduce or eliminate the production of β-globin chains, and lead to ineffective erythropoiesis, intramedullary apoptosis of erythroid precursors, and chronic hemolytic anemia. Patients with β-thal major require blood transfusions and suffer from complications such as severe anemia, chronic hemolysis with medullary expansion, hepatosplenomegaly, iron overload, heart disease and endocrine disorders.⁷⁻⁹

The only curative therapy currently available for β-hemoglobinopathies is allogeneic hematopoietic stem cell transplantation (HSCT). However, most affected individuals lack a well-matched, disease unaffected donor.¹⁰ Current treatments for SCD in developed countries include the use of hydroxyurea (HU) to induce fetal hemoglobin, which has potent anti-polymerization properties.^{11,12} HU is well tolerated and effective in many patients, but some individuals do not respond with fetal hemoglobin elevation.^{12,13} Induction of HbF has been a long-term goal for the treatment of β-hemoglobinopathies, and continued production or reactivation of HbF in adults effectively reduces many of the most serious complications of the disease phenotype, especially in SCD.¹⁴⁻¹⁸

Results of genome-wide association studies (GWAS) identified BCL11A as associated with higher levels of HbF in adults.^{19,2} Orkin and colleagues demonstrated BCL11A as a significant repressor of γ-globin expression, and multiple lines of evidence have validated the therapeutic potential of BCL11A as a molecular target.^{21,22} BCL11A is a major component regulating the physiologic globin fetal to adult switch. Clinical trials aimed at downregulating BCL11A utilize different genetic approaches such as zinc finger nucleases²³ and CRISPR-Cas9 editing show promise in elevating HbF and resolving disease complications.^{16,24,25} The inventors have utilized a unique approach employing a microRNA (miRNA)-adapted short hairpin RNA that targets BCL11A (shmiR BCL11A)²⁶⁻²⁹ to effectively and selectively reduce expression of BCL11A in erythroid cells. Studies that include gene addition with lentivirus vectors which overexpress globin in β-thalassemia patients with severe genotypes and in SCD patients, show that the effectiveness of these vectors expressing a single curative gene require several integrations per genome to produce levels of the transgene that are high enough to correct the patients' phenotype.^{29,30} Multiple integrations of vectors in the genome may increase risk of insertional mutagenesis. Thus, achieving the high-efficiency gene transfer and robust γ-globin production required to correct the SCD phenotype and β-thal remains a challenge.^{31,32} Vectors that are more effective at lower vector copy number (VCN), to achieve therapeutic levels is an important goal of current research efforts.

The zinc-finger protein 410 (ZNF410) is a novel HbF repressor that was identified via transcription factor (TF) CRISPR screening. ZNF410 does not directly bind to the genes that encode γ-globin, rather it binds and increases the transcription of the chromodomain Helicase DNA Binding Protein 4 (CHD4) gene, which encodes the nucleosome remodeling and deacetylase (NuRD) complex ATP-dependent nucleosome remodeler. NuRD itself is required for HbF repression and is in part independent of BCL11A.^{33,34} The inventors hypothesized that simultaneous reduction of both ZNF410 and BCL11A would have additive effects on fetal hemoglobin induction. The inventors generated a novel shmiR targeting ZNF410 and subsequently combined a BCL11A shmiR and a ZNF410 shmiR into the same erythroid-specific lentivirus vector to express these two shmiRs simultaneously. The combined double shmiR vector increases further the induction of HbF in erythrocytes compared to the BCL11A single shmiR vector currently in clinical trials. It was demonstrated that the double shmiR lentivirus vector is more effective in inhibiting sickling and restoring globin chain balance in erythroid cells derived from hematopoietic stem and progenitor cells (HSPCs) from SCD and β-thalassemia patients in vitro. The double shmiR lentivirus vector also more effectively attenuates the hematologic phenotypes of SCD in a murine model in vivo.

### Results

Development of Lentivirus Vectors Expressing a ZNF410 shmiR.

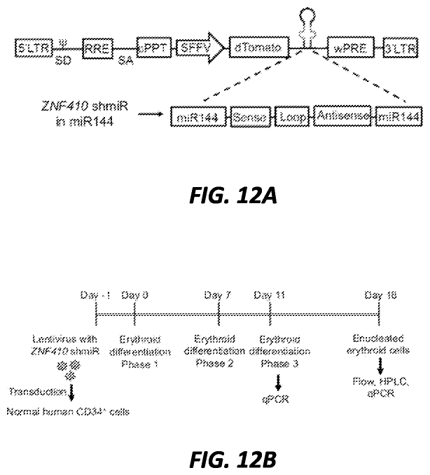

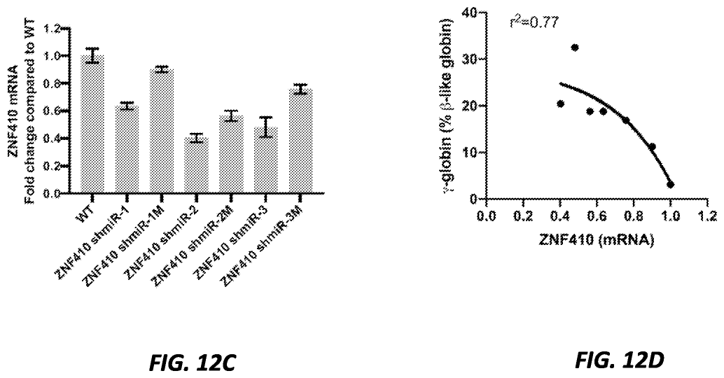

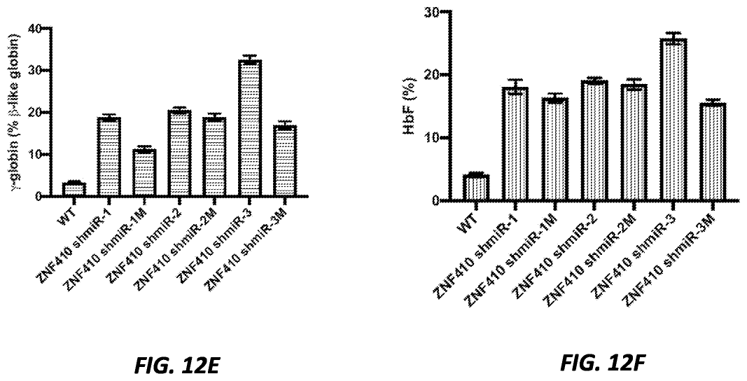

The inventors designed three shRNAs targeting the ZNF410 mRNA into pol II-driven microRNA 144-adapted shRNAs (shmiRs) expressed in lentivirus vectors from a strong SFFV ubiquitous promoter (FIG. 12A). These three shmiRs were further modified according to Guda et al.²⁷ by deleting the first four bases from the guide sequence and the addition of GCGC to the 3′ end (shmiRs modified, define M as modified). These vectors were labelled as ZNF410-shmiR-1, 1M, 2, 2M, 3, 3M. To investigate the effect of these ZNF410 shmiR vector candidates on HbF induction, CD34+ hematopoietic stem and progenitor cells (HSPCs) from healthy donors were infected with the ZNF410 shmiR lentivirus vectors and transduced cells were subjected to in vitro erythroid differentiation (FIG. 12B). Sorted, gene-modified cells demonstrated a ˜50% reduction in ZNF410 mRNA expression (FIG. 12C). There was a correlation between reduction of ZNF410 expression and induction of γ-globin (FIG. 12D). Transduction with ZNF410 shmiR-3 yielded the highest levels of γ-globin and HbF induction in erythroid progeny (FIGS. 12E, 12F). These data demonstrate effective knockdown of ZNF410 expression in erythroid cells using ZNF410 shmiR lentivirus vectors. While all ZNF410 shmiRs increased γ-globin and HbF expression in CD34-derived erythroid cells, ZNF410 shmiR-3 was utilized in subsequent development of a double shmiR vector.

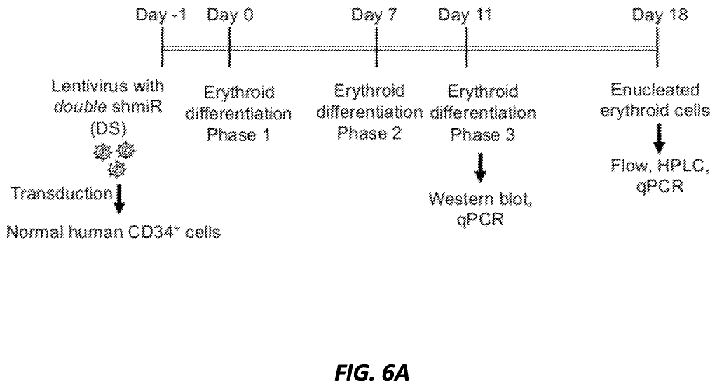

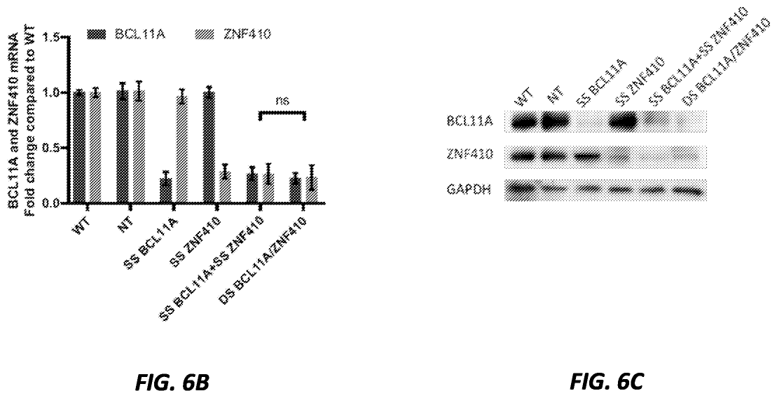

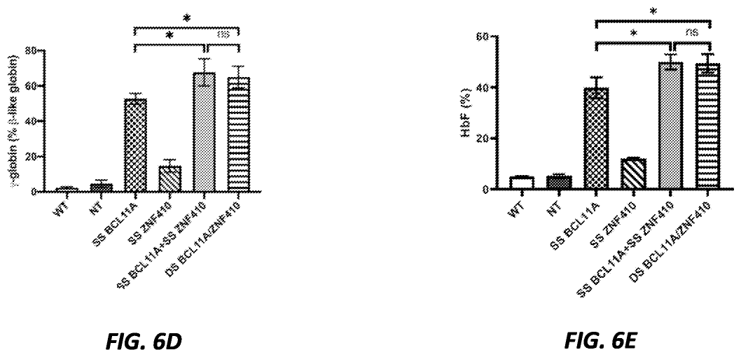

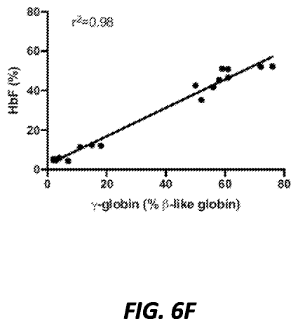

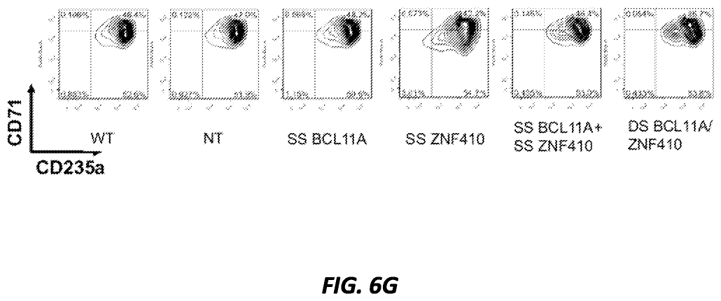

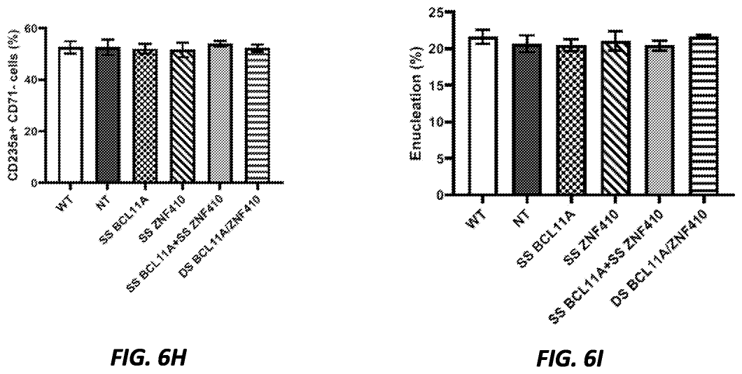

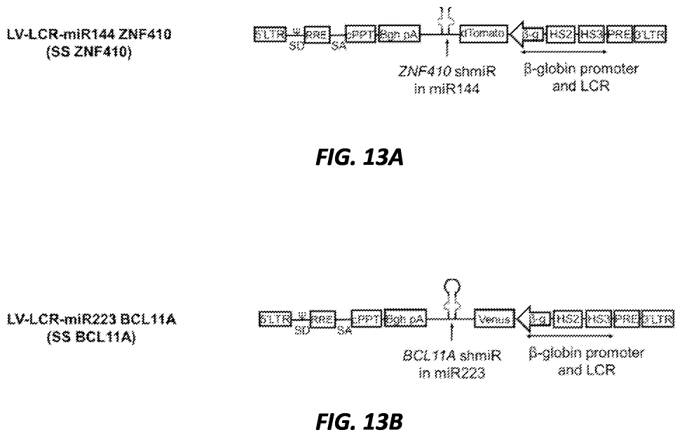

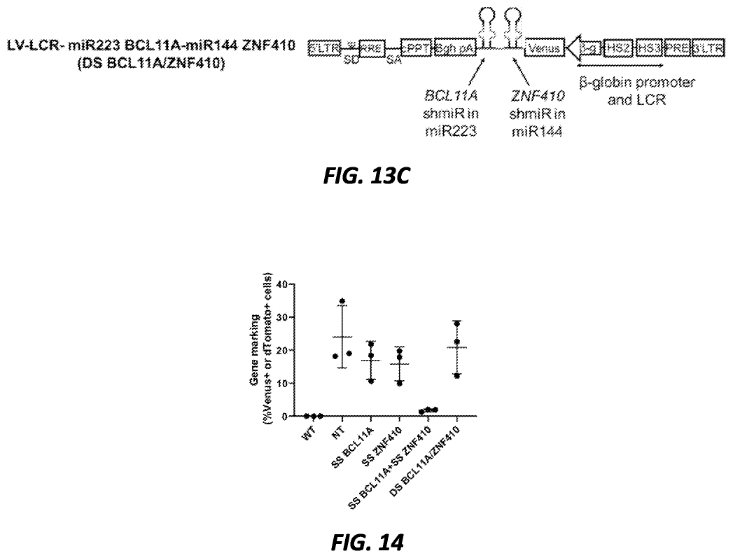

A double shmiR vector targeting both ZNF410 and BCL11A efficiently knocks down expression of both genes. The inventors hypothesized that simultaneous knockdown of BCL11A and ZNF410 using lentivirus vectors could further enhance HbF induction in erythroid cells. To test this hypothesis, CD34+ cells were first transduced concurrently with individual vectors targeting both genes. To generate double transduced cells, the inventors utilized the lentivirus vector containing ZNF410 shmiR with a Tomato fluorescence reporter (LV-LCR-miR144 ZNF410, subsequently termed SS ZNF410, FIG. 13A) and a lentivirus vector containing BCL11A shmiR with a Venus fluorescence reporter (LV-LCR-miR223 BCL11A, termed SS BCL11A, FIG. 13B). CD34+ cells from three different healthy donors were transduced simultaneously with both vectors and the transduced cells were kept in culture for 18 days under conditions supporting erythroid differentiation (FIG. 6A). Approximately 1.7% Venus and Tomato double positive gene marked cells were obtained. As seen in FIGS. 6B and 6C, cells transduced with both vectors (designated SS BCL11A+SS ZNF 410 in figures) demonstrated decreases in both BCL11A and ZNF410 mRNA and an incremental increase in γ-globin and HbF expression (FIG. 6D, 6E), confirming an additive effect on HbF induction by simultaneously targeting both BCL11A and ZNF410.

Next, to improve efficiency of transduction of target cells, a lentivirus vector incorporating both the BCL11A shmiR and ZNF410 shmiR cassettes, and the Venus fluorescent reporter under transcriptional control of the minimal β-globin proximal promoter linked to hypersensitive sites 2 and 3 (HS2 and HS3) of the β-globin locus control region (LCR), named LV-LCR-miR223 BCL11A-miR144 ZNF410 (DS BCL11A/ZNF410) (FIG. 13C) was constructed. To characterize the performance of DS BCL11A/ZNF410, the inventors transduced mobilized human peripheral blood (mPB) CD34+ cells from three different healthy donors with the double shmiR vector (designated DS BCL11A/ZNF410 in the figures). The inventors observed 24%, 16.9%, 15.8%, and 20.9% gene-marked cells after transduction with non-targeting (NT), SS BCL11A, SS ZNF410, and DS BCL11A/ZNF410 vectors, respectively (FIG. 14), representing a 10-fold enhanced transduction efficiency of DS BCL11A/ZNF410 vector over simultaneous transduction with two vectors. Transduced cells were sorted for analysis of BCL11A and ZNF410 mRNA and protein expression in erythroid cells derived from transduced CD34+ HSPCs. The use of a double shmiR led to an equivalent and simultaneous reduction of BCL11A mRNA by 80% and of ZNF410 mRNA by 70% (FIG. 6B). Western blot confirmed equivalent reduction in BCL11A and ZNF410 protein (FIG. 6C) as a result of transduction with the double shmiR vector compared to single shmiR vectors targeting each gene individually.

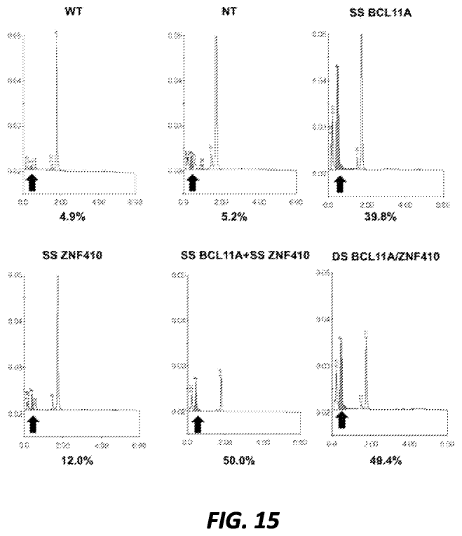

DS BCL11A/ZNF410 effectively induces HbF expression in human erythroid cells in vitro. Consistent with a reduction in mRNA and protein of BCL11A and ZNF410, erythroid cells derived from CD34+ cells transduced with the double shmiR induced significantly more γ-globin and HbF than the induction with knockdown of either gene alone (p<0.05 DS vs SS) and a similar level compared with cells derived after simultaneous transduction with both SS BCL11A shmiR plus SS ZNF410 shmiR vectors (p=ns) (FIGS. 6D, 6E, 15). Specifically, DS BCL11A/ZNF410 showed 49.4% HbF expression compared to 39.8% HbF induction after transduction with the SS BCL11A vector (p<0.05 SS vs DS). γ-globin mRNA and HbF levels were highly correlated in differentiated cells (FIG. 6F), allowing for the extrapolation of HbF based on γ-globin RT-qPCR in this experimental setting. During erythroid maturation cultures, shmiR transduced HSPCs showed normal terminal erythroid maturation based on immunophenotype and enucleation frequency (FIGS. 6G, 6H, 6I). Together, these results indicate that DS BCL11A/ZNF410 can efficiently and simultaneously knockdown BCL11A and ZNF410 and further enhance HbF induction without adverse effects on erythroid differentiation and enucleation.

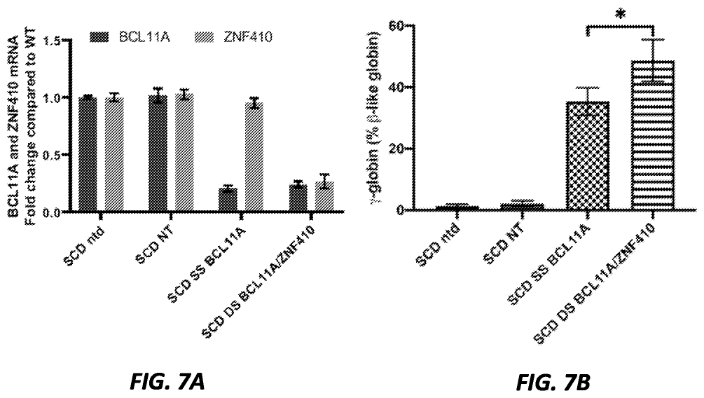

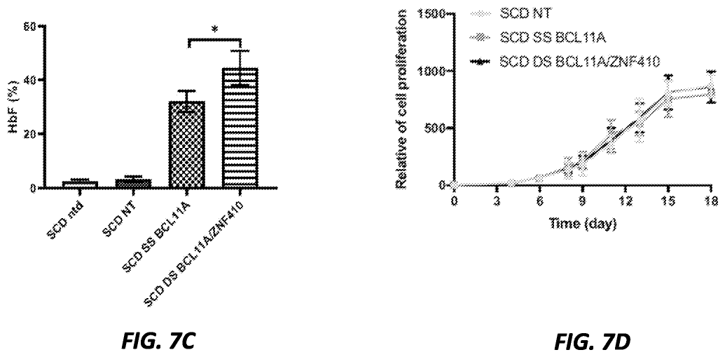

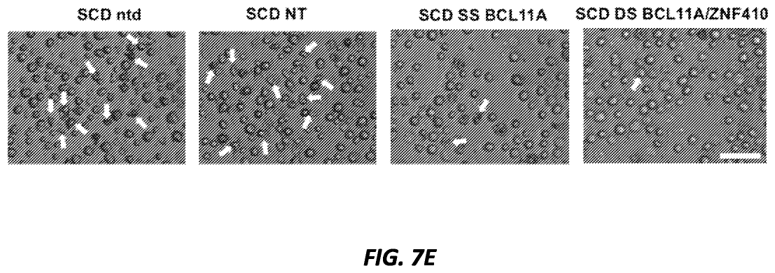

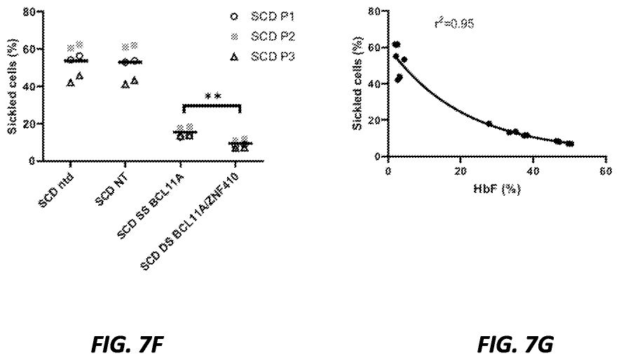

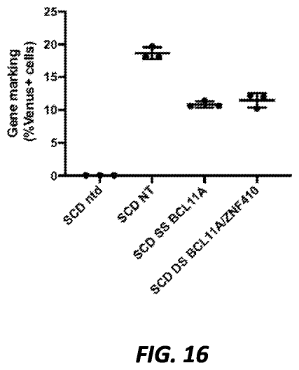

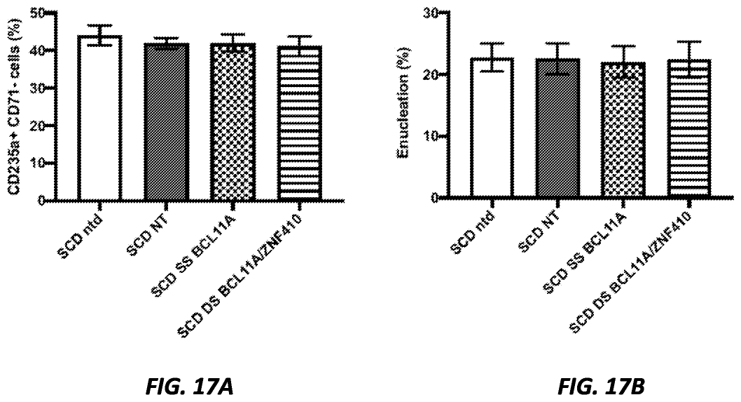

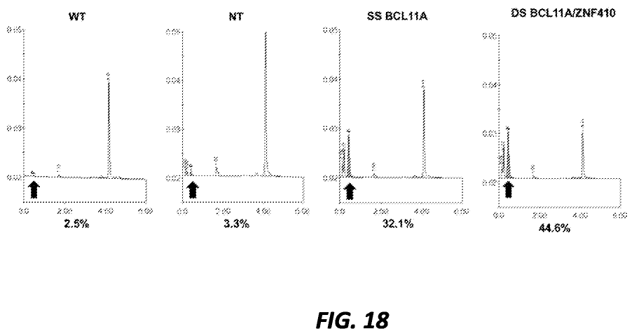

DS BCL11A/ZNF410 more effectively modify patient cells and disease cellular phenotypes than SS BCL11A shmiR vector. The inventors next evaluated the potential therapeutic impact of higher HbF induction by DS BCL11A/ZNF410 after transduction of primary HSPCs from patients with sickle cell disease (SCD) and β-thalassemia. Peripheral blood CD34+ cells from three different SCD donors were utilized for transduction with SS BCL11A and DS BCL11A/ZNF410 vectors. NT or untransduced cells served as controls. The inventors observed 10.5% and 11.5% gene marked cells after transduction with SS BCL11A and DS BCL11A/ZNF410, respectively (FIG. 16). Sorted gene-marked cells were differentiated into erythrocytes in vitro for further analysis. As demonstrated previously in normal cells, transduction with the double shmiR vector led to simultaneous knockdown of BCL11A and ZNF410 mRNA (FIG. 7A). As seen in FIG. 7A, in these cells, knockdown of BCL11A was equally effective with the double shmiR vector as with the single shmiR targeting BCL11A. Simultaneous knockdown of BCL11A and ZNF410 had no effect on erythroid cell differentiation and enucleation (FIGS. 17A-17B). After transduction and erythroid cell differentiation, the mean γ-globin induction was 48.7% and 35.3% for DS BCL11A/ZNF410 and SS BCL11A, respectively (FIG. 17B). Similarly, HbF induction was higher after transduction with the double shmiR vector. Compared to NT transduced erythroid cells which showed 3.3% HbF, DS BCL11A/ZNF410 and SS BCL11A demonstrated 44.6% and 32.1% HbF, respectively (p<0.05 DS vs SS) (FIGS. 7C, 18). And the cell proliferation result indicates that BCL11A and ZNF410 knockdown have no effect on survival (FIG. 7D). After sodium metabisulfite (MBS) treatment, the inventors observed significantly fewer sickled cells in erythrocytes derived from cells transduced with the DS BCL11A/ZNF410 vector compared with SS BCL11A, 8.0 vs 14.9% (p<0.01) (FIGS. 7E, 7F). As expected, untransduced or NT transduced erythroid cells showed robust sickling of 49.5% and 50.4%, respectively. There was a strong inverse correlation (r²=0.95) between HbF level and the percentage of sickled erythroid cells, again validating the anti-sickling effect of high levels of HbF (FIG. 7G).

Taken together, these data indicate that simultaneous knockdown of BCL11A and ZNF410 in SCD cells is feasible, further enhances HbF induction compared to knockdown of either gene alone and that the increment in HbF induction is physiologically relevant in preventing the cellular phenotype of erythrocyte sickling.

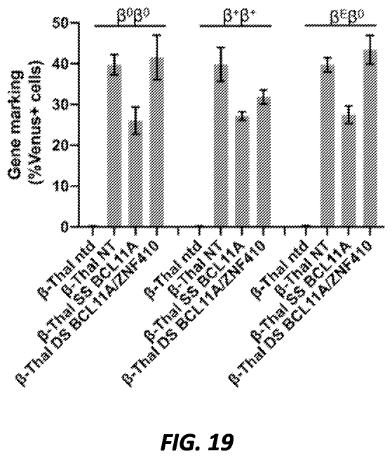

To further evaluate the potential translational value of simultaneous knockdown of BCL11A and ZNF410 using the double shmiR vector, the inventors studied cells from β-thalassemia patients. The inventors transduced peripheral blood CD34+ HSPCs from three different patients with β⁰β⁰, β⁺β⁺, β^{E}β⁰ thalassemia genotypes. Transduction efficiencies ranged from 25% to 40% with SS BCL11A and DS BCL11A/ZNF410 vectors (FIG. 19). Sorted gene-marked cells were analyzed after erythroid differentiation.

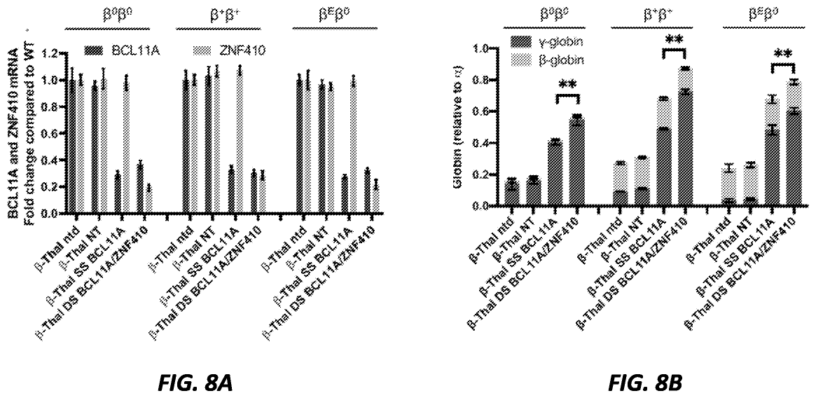

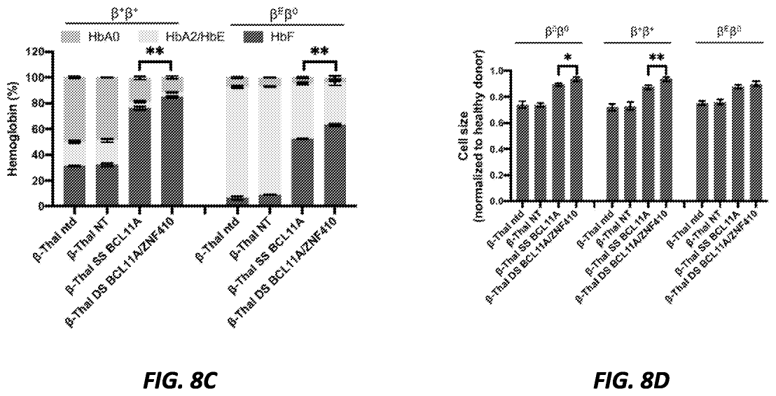

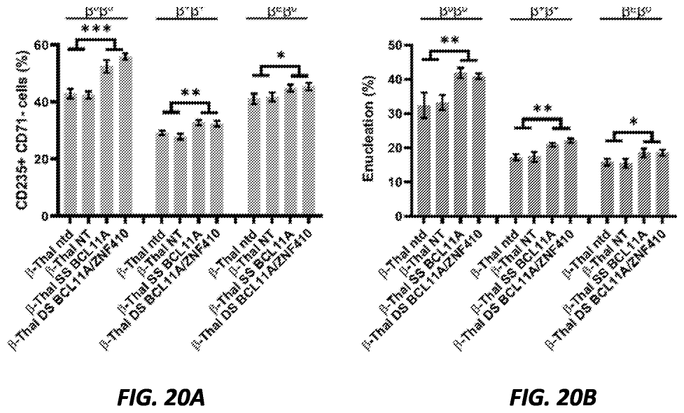

Transduction with DS BCL11A/ZNF410 led to simultaneous reduction of BCL11A and ZNF410 mRNA expression in cells derived from each donor, while transduction with SS BCL11A led to knockdown of BCL11A mRNA expression alone (FIG. 8A). In each donor, DS BCL11A/ZNF410 transduced erythroid cells showed significantly higher γ-globin and HbF induction compared to SS BCL11A cells (p<0.01) (FIGS. 8B, 8C). It was hypothesized that therapeutically relevant amelioration of globin chain imbalance, the pathophysiologic underpinning of β-thalassemia, would result in improvement of terminal erythroid maturation and reduced microcytosis. After shmiR vector transduction, a higher frequency of erythroid cell differentiation and enucleation was observed with both SS BCL11A and DS BCL11A/ZNF410 vector-transduced β-thalassemia erythroid cells than control, NT transduced cells (p<0.01) (FIGS. 20A-20B). Reduced microcytosis of enucleated β-thalassemia erythroid cells was also observed. Cell size of NT transduced group was 74% that of healthy donors, while SS BCL11A and DS BCL11A/ZNF410 were significantly larger at 88% and 91% of healthy donor, respectively, (FIG. 8D), consistent with improved erythropoiesis. Together, these data indicate that DS BCL11A/ZNF410 vector enhances HbF induction in 3-thalassemia erythrocytes and more effectively modifies some cellular β-thalassemia phenotypes.

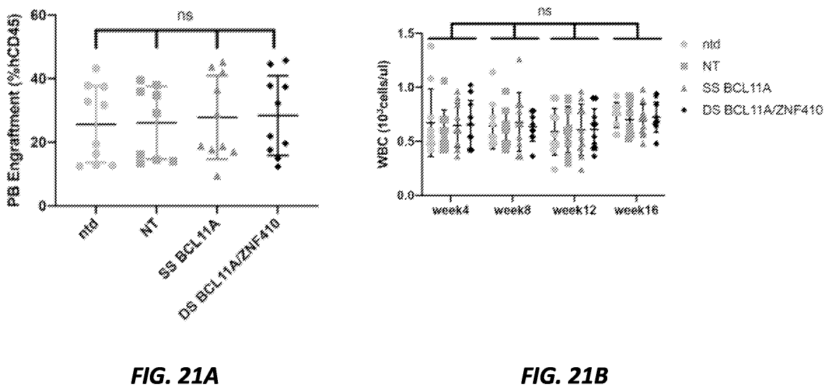

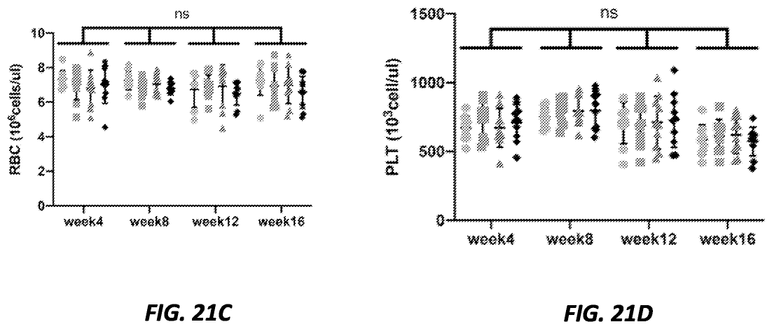

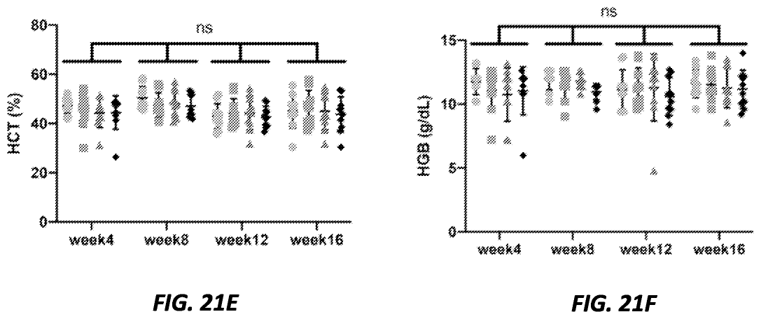

DS BCL11A/ZNF410 gene-modified cells engraft immunodeficient mice and lead to higher levels of γ-globin induction than the SS BCL11A shmiR. It was next determined if erythroid cells derived from engrafted human transduced CD34+ cells showed target gene knockdown and HbF induction. Healthy donor CD34+ cells transduced with SS BCL11A or DS BCL11A/ZNF410 were transplanted into NBSGW immunodeficient mice. NT transduced cells and untransduced CD34+ cells served as controls. Mice were bled at weeks 4, 8, 12, and 16 to analyze the engraftment of human cells. Engraftment was calculated as percentage of human CD45+ cells in the total human and murine CD45+ cell populations. The engraftment was ˜₃₀% and similar among shmiR transduced and untransduced groups (FIG. 21A). No significant differences were found between treatment groups in peripheral blood white blood cell (WBC), and platelet (PLT) counts, red blood cell (RBC) or hemoglobin (HGB) and hematocrit (HCT) (FIGS. S21B-21F) indicating that the shmiR vectors have no significant effect on hematopoietic function of transplant recipient.

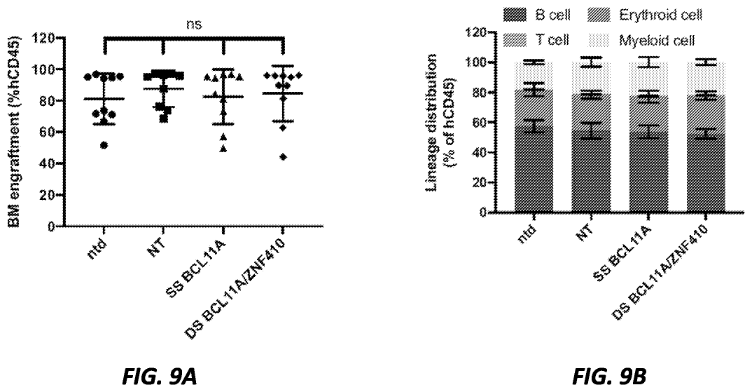

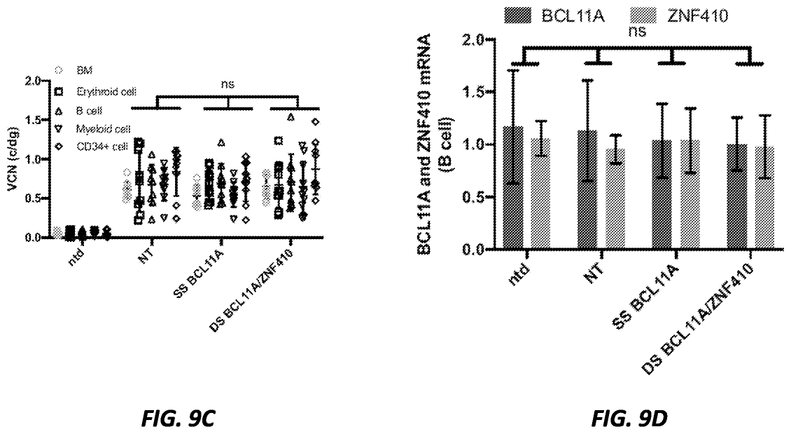

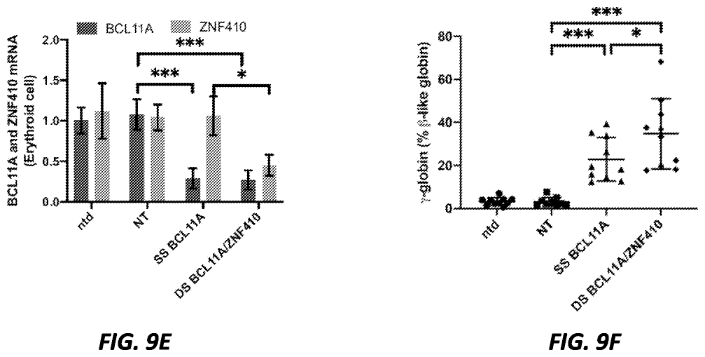

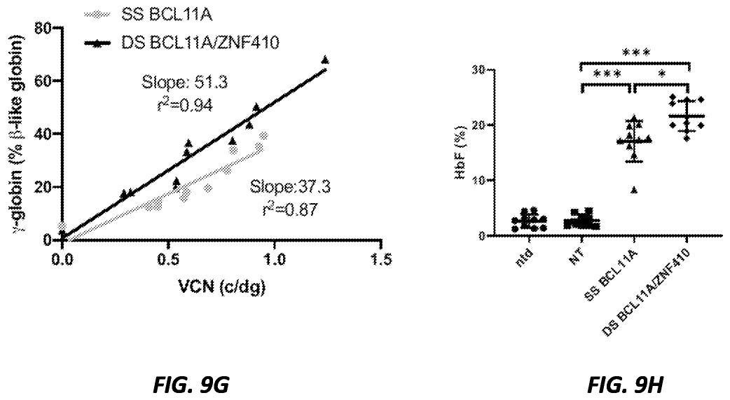

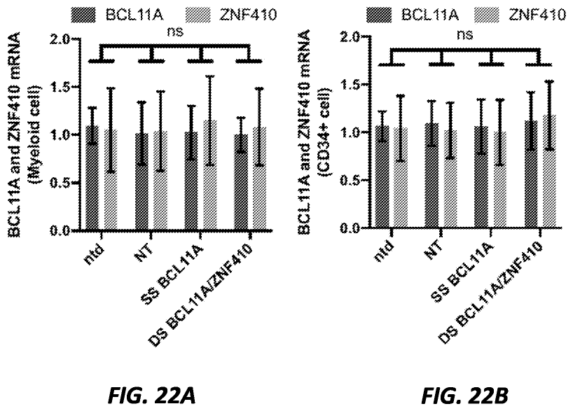

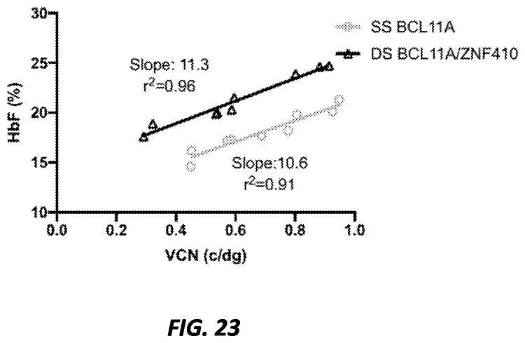

At 16 weeks post engraftment BM cells were collected and analyzed. The engraftment of untransduced and shmiR transduced CD34+ HSPCs was 80% and similar between all groups (FIG. 9A). Lineage distribution of erythroid cell, B cells, T cells and myeloid cells within the bone marrow was also similar (FIG. 9B). Vector copy number (VCN) in total BM and lineage-purified cells was similar between each experimental group (FIG. 9C). In the engrafted BM cells, there was no reduction in BCL11A or ZNF410 mRNA expression levels in B cells (FIG. 9D), myeloid cells (FIG. 22A) or CD34+ cells (FIG. 22B), confirming the lack of knockdown in non-targeted lineages. Both BCL11A and ZNF410 mRNA expression was reduced in erythroid cells in mice engrafted with DS BCL11A/ZNF410 transduced CD34+ cells, while only BCL11A was reduced in mice engrafted with SS BCL11A (FIG. 9E), demonstrating the erythroid specificity of these vectors. Consistent with the knockdown at the mRNA level, in human erythroid cells derived from the bone marrow, the inventors observed robust induction of γ-globin, increasing from 3.5 to 22.8% with SS BCL11A and a significantly higher induction to 33.3% with DS BCL11A/ZNF410 (p<0.05) (FIG. 9F). The inventors normalized γ-globin expression to erythroid cell VCN for each shmiR vector. As shown in FIG. 9G, DS BCL11A/ZNF410 led to a higher γ-globin expression than SS BCL11A shmiR when normalized to VCN, as shown by the increased slope of the fitted line. HbF induction in erythroid cells differentiated in vitro from BM hCD34+ cells were also analyzed by HPLC (FIG. 9H). Transduction with the DS BCL11A/ZNF410 vector increased HbF to a significantly higher level than SS BCL11A (HbF=21.6% vs 17.1%, p<0.05). Normalized to VCN in erythroid cells, HbF expression, showed trend towards higher HbF expression in DS BCL11A/ZNF410 transduced cells compared with BCL11A shmiR at same VCN, as shown by the increased slope of the fitted line, but this difference failed to reach significance (FIG. 23).

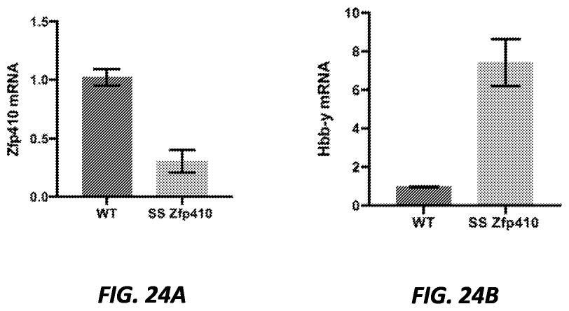

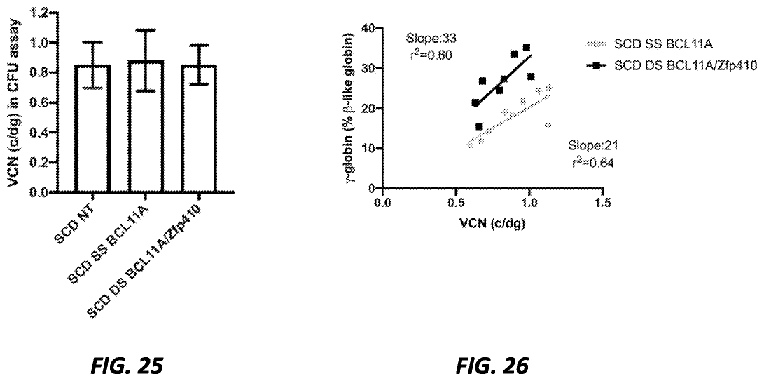

Genetic modification of Berkeley SCD HSCs with double shmiR vector leads to improvement of disease-associated hematological parameters. It was next determined if the double shmiR vector could ameliorate characteristic SCD disease parameters in a transplantation model utilizing Berkeley SCD (BERK-SCD) mouse HSCs. The inventors designed a DS BCL11A/Zfp410 for double knockdown in murine BM cells, replacing the ZNF410 shmiR with a Zfp410 shmiR targeting the mouse sequence in DS BCL11A/ZNF410, and Zfp410 shmiR successfully knockdown Zfp410 and induce Hbb-γ mRNA expression in MEL cells (FIG. 24). Lineage-negative CD45.1+BM cells from BERK-SCD mice were transduced with DS BCL11A/Zfp410, SS BCL11A vector, NT vector or left untransduced (as an unmanipulated disease control) and resulting transduced cells were transplanted into lethally irradiated CD45.2+BL/6 recipient animals. An additional control group received untransduced cells isolated from WT BoyJ animals in order to quantitate maximal correction of phenotype. In vitro VCN was determined by colony forming unit (CFU) assay on progenitor-derived colonies from the cell product 14 days after transduction (FIG. 25).

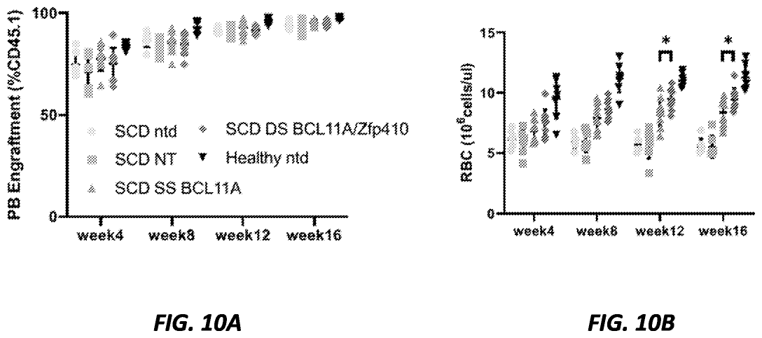

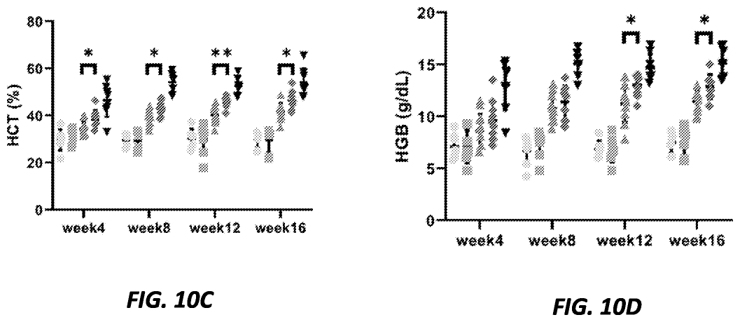

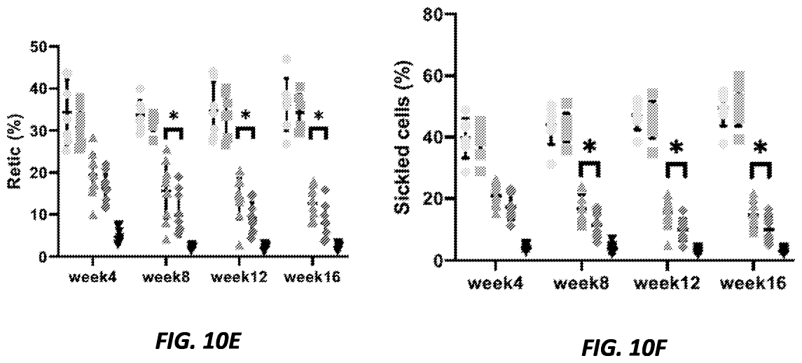

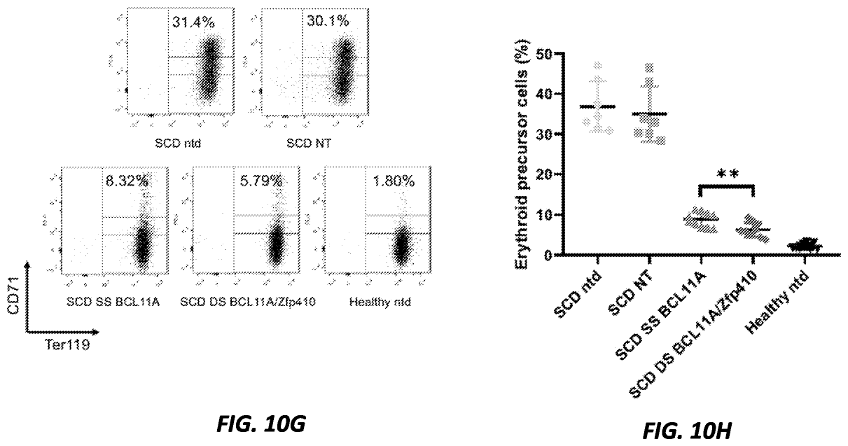

Engraftment as determined by flow cytometric enumeration of CD45.1+ donor cells and RBC counts, HGB/HCT, concentrations were measured on PB samples acquired 4-, 8-, 12-, and 16-weeks post-transplantation. PB engraftment was greater than 95% at week 16 (FIG. 10A) and similar in all recipient groups receiving BERK-SCD HSCs, indicating that transduction did not alter the fitness of transplanted cells. To analyze SCD phenotype, RBC, HGB/HCT, reticulocyte level and the percentages of sickled erythrocytes were measured. Engraftment of untransduced BERK-SCD HSCs was associated with significantly lower RBC, HGB and HCT levels, increased reticulocytes and sickled cells, indicative of severe hemolytic anemia, supporting the validity of the experimental model.

Mice transplanted with BERK-SCD HSCs transduced with the NT vector showed similar results compared with the untransduced group, while SS BCL11A and DS BCL11A/Zfp410 transduced groups showed significant improvements in all blood parameters. The inventors observed that the animals engrafted with cells transduced with the DS BCL11A/Zfp410 vector demonstrated significant improvement in hematologic parameters compared with animals engrafted with cells transduced with the SS BCL11A shmiR vector. After engraftment with DS BCL11A/Zfp410 transduced cells, the RBC count at week 16 was 9.4×10⁶ cell/ul compared with 8.4×10⁶ cell/ul for SS BCL11A (FIG. 10B); HCT percentage was 45% in DS BCL11A/Zfp410 compared with 42% in SS BCL11A transduced group (p<0.05) (FIG. 10C), HGB expression level was 12.8 g/dL of DS BCL11A/Zfp410 compared with 11.4 g/dL of SS BCL11A (p<0.05) (FIG. 10D). Reticulocyte counts (FIG. 10E) were decreased from 35% in the NT group to 8% in the DS BCL11A/Zfp410 transduced group which was significantly lower than the SS BCL11A transduced group at 12% (p<0.05), indicative of reduced erythropoietic stress because of reduced hemolysis and improved RBC survival.

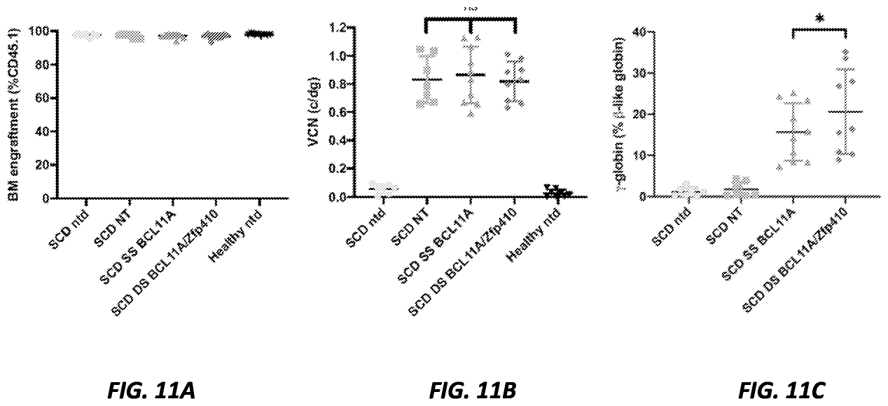

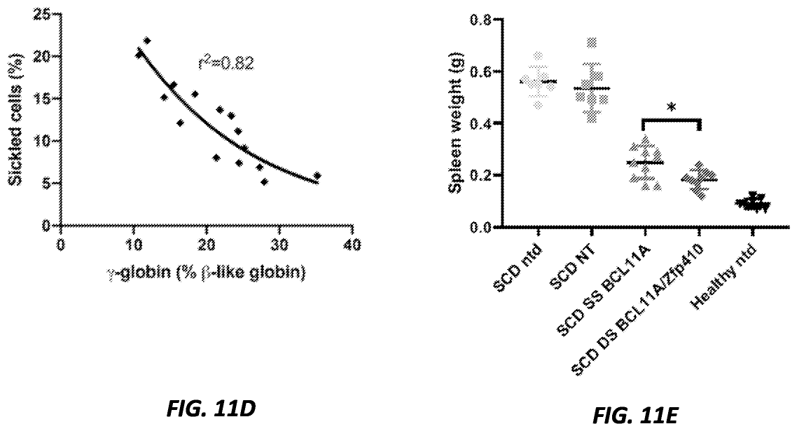

The number of irreversibly sickled RBCs after sodium metabisulfite treatment of collected PB was next tested. PB from animals transplanted with the DS BCL11A/Zfp410 vector transduced cells demonstrated significantly fewer sickled cells compared with SS BCL11A group. PB taken from mice transplanted with untransduced or NT shmiR transduced HSPCs at week 16 displayed 49.5% and 50.4% irreversibly sickled RBC, respectively while mice transplanted with SS BCL11A or DS BCL11A/Zfp410 transduced HSPCs displayed a decrease in percentage of sickled cells in PB after treatment with MBS with 14.8%, and 10.0%, respectively (p<0.05) (FIG. 10F). Consistent with decreasing rates of hemolysis and lower reticulocyte and sickled cells counts, the inventors also observed a striking reduction in circulating erythrocyte precursors between treatment groups, with a significant difference between SS BCL11A and DS BCL11A/Zfp410 groups. The frequency of CD71+Ter119+erythroid precursors in the peripheral blood at week 16 (FIGS. 10G, 10H), was reduced from 35% (untransduced and NT groups) to 8.8% in the SS BCL11A group, and 6.2% in DS BCL11A/Zfp410 group. These data show that transplantation of DS BCL11A/Zfp410 transduced SCD HSPCs leads to durable production of RBCs that are resistant to sickling both in vivo and in vitro. After sacrifice, additional studies were performed on spleen and BM. BM engraftment was >97% (FIG. 11A) and similar in all recipient groups receiving BERK-SCD HSCs. The average BM VCN was similar among all experimental arms (FIG. 11B) and matched the average VCN of in vitro cultured cells before transplantation. To determine whether shmiR vectors retained the ability to achieve therapeutic levels of expression in BM cells, the inventors measured γ-globin by RT-qPCR. The DS BCL11A/Zfp410 treated group showed significantly higher γ-globin expression than SS BCL11A treated group (p<0.05). Average levels of γ-globin were 1.7%, 18.5%, and 25.4% for NT group, SS BCL11A, and DS BCL11A/Zfp410, respectively (FIG. 11C). To compare the difference in expression per vector genome between SS BCL11A and DS BCL11A/Zfp410, the inventors normalized γ-globin expression to BM VCN for different shmiR vectors. As seen in FIG. 26, erythrocytes derived from DSBCL11A/Zfp410 transduced cells demonstrated a trend towards higher normalized γ-globin expression compared with SS BCL11 A as shown by the increased slope of the fitted line, but this difference failed to reach significance. A strong negative correlation was observed between the reduction of sickled RBCs in PB after MBS treatment and induction of γ-globin in BM erythroid cells (FIG. 11D).

The mitigation of the sickling phenotype was also associated with reduced spleen size, with the DS BCL11A/Zfp410 treated group showing significantly less spleen mass than SS BCL11A treated group (p<0.05). The average spleen mass of SS BCL11A vector and DS BCL11A/Zfp410 vector group decreased to 0.25±0.06 g and 0.18±0.04 g, respectively compared to 0.53±0.06 g in the NT group, while mice that received healthy cells showed spleen weights of 0.09±0.02 g (FIG. 11E). Thus, the persistence of DS BCL11/Zfp410 transduced cells leads to a more robust rescue of all SCD cellular phenotypes examined.

The downregulation of BCL11A in erythroid cells leads to sustained reactivation of γ-globin, the production of HbF resulting in reduced polymerization of sickle-containing hemoglobin, and significant mitigation of the hematologic effects of SCD in a murine model.^{21,22} The inventors previously reported a lentiviral vector that mediates knockdown of BCL11A via a short hairpin RNA embedded in a microRNA (shmiR) expressed selectively in erythroid cells.^{26,27} Subsequent clinical studies have used this vector design to validate BCL11A as a therapeutic target in sickle cell disease in humans.^{15,28} Additional clinical studies underway³⁵⁻³⁷ add back copies of a mutated form of adult β-globin which is resistant to sickling. However, to be maximally effective, these gene transfer strategies require the sustained engraftment of a large number of transduced HSCs¹⁰ to effect polyclonal reconstitution. The approach of adding back additional globin gene sequences also appears to require a higher number of vector insertions/cell to effect maximal transgene expression. High vector copy numbers (VCN) may increase genotoxic risks, an outcome of particular relevance in SCD patients who may have an increased risk of developing hematological malignancies compared to the general population.³⁸ While promising approaches aimed at improving LV-derived gene expression have been investigated,³⁹ reaching therapeutic gene expression with a low VCN is still challenging.

Induction of HbF has been a long-term goal for the treatment of β-hemoglobinopathies; its expression can be effective in reducing many of the most serious complications of the disease phenotype. Targeting BCL11A to reverse the physiological fetal to adult globin switch to increase HbF and concurrently reduce HbS has clear advantages. HbS content and the percentage of HbF are the two main modulators of clinical severity. HbF has potent anti-sickling characteristics and a level of HbF of approximately one-third of the total cellular content of hemoglobin would likely prevent HbS polymerization, while the concurrent reduction in intracellular HbS further attenuates the tendency for polymer formation.⁴⁰⁻⁴⁴ Induction of high levels of HbF distributed broadly in red blood cells is the most promising approach to the pharmacologic treatment of sickle cell anemia because it targets the proximal pathophysiologic trigger of disease.⁴⁵ In previously published work,^{26,28} the use of a lentivirus shmiR vector targeting BCL11A leads to a significant induction of HbF at VCN <1, a clear advantage of this approach. However, due to the complex biology of gamma globin repression,⁴⁶ maximal induction of HbF may not be obtained in all cells using this approach.

Vinjamur et al. previously have identified ZNF410 as a BCL11 A-independent HbF repressor that specifically activates the expression of CHD4.^{33,34} In this study, the inventors developed erythroid-specific double shmiR-expressing vectors that simultaneously knockdown two genes, BCL11A and ZNF410, and consistently enhance HbF induction an additional ˜10% compared to knockdown of BCL11A alone. One advantage of this approach is the efficiency of transduction of rare and difficult to transduce hematopoietic stem cells compared to simultaneous transduction with two different vectors. Thus as expected, the inventors show in xenograft experiments utilizing human CD34+ cells the transduction efficiency of DS BCL11A/ZNF410 on CD34+ and progeny B cells, myeloid and erythroid cells was similar to that of SS BCL11A. In addition, a positive correlation was observed between HbF induction and VCNs in erythroid cells of DS BCL11A/ZNF410-transplanted animals and the ratios of HbF induction per VCN were higher in DS BCL11A/ZNF410 transplanted mice compared to SS BCL11A transplanted animals.

The enhanced induction of HbF expression per red cell may thus lead to a more effective induction of total HbF while maintaining the safety of lower VCN/cell in humans as demonstrated here in the SCD mouse model and in the xenograft models using human cells. The vector reported here uniquely combines two shmiR in the same vector to induce HbF. HbF expression can be induced by knockdown of not only BCL11A and ZNF410 but also other regulatory genes, such as LRF⁴⁷ and POGZ,⁴⁸ so additional combinatorial approaches are possible. This strategy could also be combined with other strategies to achieve maximal mitigation of sickle hemoglobin polymerization. For instance, Uchida et al. ³² combined BCL11A knockdown and thEpoR coexpression for improved HSC-targeted gene therapy for hemoglobin disorders in humans. The co-expression of a BCL11 A shmiR and a therapeutic β-globin gene in one vector to enhance functional hemoglobin production has recently been described.²⁹ In addition, recent genome editing technologies allow for development of other HbF induction methods, such as targeting the erythroid-specific BCL11A enhancer,⁴⁹ a BCL11A binding site in γ-globin promoters similar to the Greek variant of HPFH,⁵⁰ and inducing a large deletion in the δ-globin gene mimicking Sicilian HPFH.⁵¹ Ramadier et al. developed therapeutic approaches combining LV-based gene addition and CRISPR-Cas9 strategies aimed to increase the incorporation of an anti-sickling globin (AS3) and induce the expression of HbF.⁵²

Multiple of these strategies appear effective but which is safest or most effective in humans is still unknown. However, the DS shmiR vector approach demonstrated here offers several potential advantages. First, lentiviral shmiR for HbF induction leaves HBBS alleles intact, which largely avoids DSBs generated by nucleases such as Cas9 that lead to uncontrolled mixtures of indels at the target site as well as the potential for large deletions, translocations, chromosomal loss, chromothripsis, and activation of the p53 DNA damage response.⁵³⁻⁵⁵ Second, since the DS BCL11A/ZNF410 uses a physiologic switch, this approach concurrently and proportionately reduces the concentration of sickle hemoglobin in RBCs, the primary determinant of pathogenic hemoglobin polymerization, more effectively than targeting BCL11A alone. Although other approaches including a single BCL11A shmiR vector with anti-sickling HBB transgene vector can decrease the fraction of βS in erythroid progeny by 30-70%,^{26,56} the inventors achieved even greater βS percentage reduction in erythroid populations by DS BCL11A/ZNF410 shmiR without changing red blood cell differentiation kinetics. Double shmiR-transduced patient derived CD34+ cells thus provide a promising basis for autologous treatment for SCD and β-thalassemia. This strategy can also be combined with other induction strategies to achieve sustained HbF induction or express an anti-sickling HBB transgene.

In summary, the inventors developed erythroid-specific lentiviral vectors encoding a double shmiR targeting two repressors of gamma globin allowing for an enhanced HbF induction at a similar VCN compared to targeting only BCL11A. The double shmiR vector can effectively knockdown target genes in cells at the same time with high transduction efficiency, which can be used as a model to target multiple gene products simultaneously and efficiently. The inventors report the functional characterization of a novel and efficient LV expressing double shmiR in clinically relevant cells from SCD and β-thalassemia patients as part of a program of work aimed at clinical translation of an effective LV-based gene therapy approach for these diseases. Finally, multi-shmiR LVs can have a wider range of potential applications in this field since multiple shmiRs could be exploited as an approach in other diseases with complex pathophysiology affecting multiple pathways.

### Methods

Construction of shmiR constructs The generation of mir223 BCL11A vectors has been previously described.²⁷ For mir144 ZNF410 vectors, to introduce desired sequence substitution, the inventors used sets of reverse-oriented primers with the replaced sequence to PCR amplify the mir223 BCL11A plasmid backbone by Q5® Site-Directed Mutagenesis Kit (New England Biolabs, Ipswich, MA) and then phosphorylated and ligated the resultant linearized plasmids. LV-LCR-mir223 BCL11A-mir144 ZNF410 vector was created by using NEBuilder HiFi DNA Assembly kit (New England Biolabs, Ipswich, MA) to fuse together PCR amplified mir144 ZNF410 shmiR fragment to mir223 BCL11A linearized plasmids that digested with Mull (New England Biolabs, Ipswich, MA). mi223 NT vectors with non-target shmiR cassette were used as controls.

Virus production and titration Lentiviral vector supernatants were produced by adding 10 μg of lentiviral vector, 5 μg of gag-pol, 2.5 μg of rev and 2.5 μg of VSVG packaging plasmids into HEK 293T cells grown in 10-cm plates. Plasmids were mixed with 1 ml DMEM (Cytiva, Marlborough, MA) and 60 μl of 1 mg/ml linear PEI (Polysciences, Warrington, PA), incubated for 15-20 minutes at room temperature, and added to the culture dish. The medium was changed 14 hours later and virus supernatants were collected 48 and 72 hours post transfection, filtered through a 0.45 μm membrane (Corning, New York, NY), and then concentrated by ultracentrifugation at 23,000 rpm for 2 hours in a Beckmann XL-90 centrifuge with SW-28 swinging buckets.

Infectious titers were determined on mouse erythroid leukemia (MEL) cells by applying serial dilutions of vector supernatant followed by erythroid differentiation for 4 days in RPMI (Cytiva, Marlborough, MA) supplemented with 1.25% DMSO (Sigma-Aldrich, St Louis, MO), and 5% fetal calf serum (Summerlin Scientific Hampton, NH).

Cell culture 293T and MEL cells were maintained in DMEM (Cytiva, Marlborough, MA) or RPMI medium (Cytiva, Marlborough, MA) supplemented with 10% fetal calf serum (Summerlin Scientific Hampton, NH), 1% penicillin-streptomycin (Thermo Fisher, Waltham, MA), respectively.

Transduction of human CD34+ cells Human CD34+ HSPCs from mobilized peripheral blood of anonymized healthy donors were obtained from Fred Hutchinson Cancer Research Center, Seattle, Washington. Sickle cell disease patient and β-thalassemia patient CD34+ HSPCs were isolated from unmobilized peripheral blood following Boston Children's Hospital institutional review board (IRB) approval and informed patient consent. The CD34+ HSPCs were enriched using the Miltenyi CD34 Microbead kit (Miltenyi Biotec, Auburn, CA). CD34+ cells were prestimulated for 44-48 hours at 1×10⁶ cells/mL in Stem Cell Growth Media (CellGenix, Portsmouth, NH) supplemented with Stem Cell Factor (SCF), FMS-like tyrosine kinase 3 ligand (FLT3L) and thrombopoietin (TPO) all from Peprotech (Rocky Hill, NJ). Cells were then enumerated and transduced with the virus at an MOI as indicated for 24 h before downstream processing.

In vitro erythroid differentiation of CD34+ cells The erythroid differentiation protocol the inventors used is based on a three-phase protocol adapted from Giarratana et al.⁵⁷ The cells were cultured in erythroid differentiation medium (EDM) consisting of Iscove modified Dulbecco's medium (Cellgro, Manassas, VA) supplemented with 1% 1-glutamine (Thermo Fisher, Waltham, MA), and 1% penicillin/streptomycin (Thermo Fisher, Waltham, MA), 330 μg/ml holo-human transferrin (Sigma-Aldrich, St Louis, MO), 10 μg/ml recombinant human insulin (Sigma-Aldrich, St Louis, MO), 2 IU/ml heparin (Sigma-Aldrich, St Louis, MO), and 5% human solvent detergent pooled plasma AB (Rhode Island Blood Center, Providence, RI), 3 IU/ml erythropoietin (Amgen, Thousand Oaks, CA). During the first phase of expansion (days 0 to 7), CD34+ cells were cultured in EDM in the presence of 10⁻⁶ mol/l hydrocortisone (Sigma-Aldrich, St Louis, MO), 100 ng/ml stem cell factor (SCF) (Peprotech, Rocky Hill, NJ), 5 ng/ml IL-3 (R&D Systems, Minneapolis, MN), as EDM-1. In the second phase (days 7 to 11), the cells were resuspended in EDM supplemented with SCF, as EDM-2. For the third phase (days 11 to 18), the cells were cultured in EDM without additional supplements, as EDM-3.

Western blot analysis On day 11 of erythroid differentiation, differentiated CD34+ cells were lysed in lysis buffer (RIPA) with phosphatase inhibitors (Santa Cruz Biotechnology, Dallas, TX). Total protein extracts were suspended in 2× Laemmli (Bio-Rad, Hercules, CA) sample buffer, boiled, and loaded onto a 10% SDS-polyacrylamide gel, then transferred to a polyvinylidene fluoride membrane (Millipore, Billerica, MA). To block nonspecific binding sites, the membranes were treated for 1 h with TBST (mixture of Tris-buffered saline and Tween-20) containing 5% milk. Membranes were next incubated overnight at 4° C. with mouse anti-BCL11A antibody (Abcam, Cambridge, MA) or rabbit Anti-ZNF410 Polyclonal Antibody (Proteintech, Rosemont, Il). After washing, the membranes were incubated for 1 h with HRP-linked anti-rabbit or anti-mouse IgG secondary antibody (Cell Signaling, Danvers, MA). The expression of GAPDH in the cells was also measured as a control.

RNA extraction and qRT-PCR Total RNA was extracted using an RNeasy micro kit (QIAGEN, Venlo, Netherlands). Reverse transcription of mRNA employed the SuperScript First-Strand Synthesis System for RT-PCR (Invitrogen, Carlsbad, CA, USA) with oligo(dT) primers. qRT-PCR was performed using the SYBR Green PCR master mix (Applied Biosystems, Foster City, CA, USA) as a detection system.

VCN Assay Genomic DNA was extracted using the QIAGEN DNeasy protocol (QIAGEN, Hilden, Germany). VCN was assessed by RT-qPCR, performed with the use of TaqMan Fast Advanced Master Mix (Applied Biosystems, Foster City, CA). VCN was calculated by using primers and probes HIV-1 PSI (forward 5′-CAGGACTCGGCTTGCTGAAG-3′ (SEQ ID NO: 29), reverse 5′-TCCCCCGCTTAATACTGACG-3′ (SEQ ID NO: 30), probe FAM-5′-CGCACGGCAAGAGGCGAGG-3′ (SEQ ID NO: 31)) as a target and the human glycosyltransferase Like Domain Containing 1 gene (GTDC1) as an internal reference standard (forward 5′-GAAGTTCAGGTTAATTAGCTGCTG-3′ (SEQ ID NO: 32), reverse 5′-T GGCACCTTAACATTTGGTTCTG-3′ (SEQ ID NO: 33), probe VIC-5′-ACGAACTTCTTGGAGTTGTTTGCT-3′ (SEQ ID NO: 34)). Standard curves were obtained by serial dilutions of a plasmid containing one copy of PSI and GTDC1 sequences. The number of PSI and GTDC1 copies in test samples was extrapolated from the standard curves.

Hemoglobin Analysis by HPLC After 18 day of differentiation, 1 million erythroid cells were lysed by using Hemolysate reagent (Helena Laboratories, Beaumont, TX), then incubated on ice for 15-20 min and vortex every 5 min. Hemolysates were prepared by centrifugation at 15,000 rpm for 5 mins and analyzed with use of the D-10 Hemoglobin Analyzer (Bio-Rad), in order to identify the hemoglobin variants HbF and HbA and determine their levels.

In vitro sickling assay At the completion of erythroid differentiation, enucleated RBCs were sorted, with use of Hoechst 33342 (5 mg/mL; Invitrogen, Carlsbad, CA), and subjected to in vitro sickling assay. Sickling was induced by adding 500 μl of freshly prepared 2% sodium metabisulfite (Sigma-Aldrich, St Louis, MO) solution prepared in PBS into enucleated cells resuspended with 500 μl of EDM-3 in a 24-well plate, followed by incubation at 37° C. for 30 min. Live cell images were acquired using a Nikon Eclipse Ti inverted microscope. More than 500 cells with irregular structure, protruding spikes, or sickle shape were counted as sickling cells.

Flow cytometry for enucleation and cell size analysis For the enucleation analysis, cells were stained with 2 μg/ml of the cell-permeable DNA dye Hoechst 33342 (Invitrogen, Carlsbad, CA) for 20 minutes at 37° C. The Hoechst 33342-negative cells were further gated for cell size analysis with Forward Scatter A parameter. The median value of forward scatter intensity normalized by data from healthy donors collected at the same time was used to characterize the cell size.

Human CD34+ HSPC transplant and flow cytometry analysis All animal experiments were approved by the Boston Children's Hospital Institutional Animal Care and Use Committee. NOD.Cg-KitW-41J Tyr+ Prkdcscid Il2rgtm1Wjl (NBSGW) mice were obtained from Jackson Laboratory (Stock 026622).

Non-irradiated NBSGW female mice (4-6 weeks of age) were infused by retro-orbital injection with 1×10⁶ virus transduced CD34+ HSPCs (resuspended in 150 μl DPBS) derived from healthy donors. Peripheral blood samples were collected at week 4, 8, 12 and 16 to measure engraftment by flow cytometry (hCD45/mCD45) and determine RBC indices. At week 16, mice were euthanized, and BM was isolated for human xenograft analysis. A portion of the BM cells was performed to erythroid differentiation in vitro. For flow cytometric analyses of BM, the following antibodies were used: hCD45, mCD45 and fixable viability dye eFluor 780 are from Thermo Fisher (Waltham, MA), hCD235a, hCD33, hCD19, hCD34 and hCD3 are from BioLegend (San Diego, CA).

In vivo experiment in SCD mouse model Lineage-negative mouse bone marrow cells were isolated by flushing femurs, tibias, and iliac crests of 6- to 8-week-old CD45.1 BoyJ (B6.SJL-Ptprca Pepcb/BoyJ) or CD45.1 Berkeley SCD mice (BEKER-SCD, JAX stock #003342) followed by lineage depletion using the Mouse Lineage Cell Depletion Kit (Miltenyi Biotec). Lin− cells were pre-stimulated at 1×10⁶ cells/mL in Stem Cell Growth Media (CellGenix) supplemented with mSCF (100 ng/mL), hTPO (100 ng/mL), mIL-3 (20 ng/mL), and hFlt3-L (100 ng/mL), all from Peprotech (Rocky Hill, NJ). Following a 24-hour prestimulation, cells were transduced at a density of 1×10⁶ cells/ml at an MOI of 40, and transduced cells were transplanted by retro-orbital injection into lethally irradiated (7+4 Gy, split dose) CD45.2 recipients 3 days after isolation. A portion of the transduced cells was used to seed a methylcellulose based CFU assay to determine the VCN in the cell products. Peripheral blood samples were collected at weeks 4, 8, 12 and 16 to measure engraftment by flow cytometry (CD45.2/CD45.1), determine RBC indices and quantitate sickled cells. At week 16, mice were euthanized, and BM cells were used to measure engraftment by flow cytometry (CD45.2/CD45.1), VCN, and mRNA expression. For flow cytometric analyses of BM the following antibodies were used: CD45.1, CD45.2, CD11b and CD3 are from BioLegend (San Diego, CA), B220, CD71, and Ter119 are from BD Pharmingen (Woburn, MA), and fixable viability dye eFluor 780 is from Thermo Fisher (Waltham, MA).

Statistical analysis All data are reported as mean±SD unless otherwise stated. Statistical analyses were performed using GraphPad Prism version 8.0 (GraphPad Software, San Diego, California, USA). The statistical significance between two averages was established using unpaired t test. When the statistical significance between three or more averages were evaluated, a one-way ANOVA was applied followed by multiple paired comparisons for normally distributed data (Tukey test). All statistical tests were two-tailed, statistical significance differences are indicated with asterisks: *p<0.05, **p<0.01, ***p<0.005 and N.S. denotes p>0.05.

### REFERENCES

1. Cavazzana, M., Antoniani, C., and Miccio, A. (2017). Gene Therapy for beta-Hemoglobinopathies. Mol. Ther. 25, 1142-1154.
2. Ghiaccio, V., Chappell, M., Rivella, S., and Breda, L. (2019). Gene Therapy for Beta-Hemoglobinopathies: Milestones, New Therapies and Challenges. Mol. Diagn. Ther. 23, 173-186.
3. Al-Saif, A. M. (2019). Gene therapy of hematological disorders: current challenges. Gene Ther. 26, 296-307.
4. Demirci, S., Uchida, N., and Tisdale, J. F. (2018). Gene therapy for sickle cell disease: An update. Cytotherapy 20, 899-910.
5. Hoban, M. D., Orkin, S. H., and Bauer, D. E. (2016). Genetic treatment of a molecular disorder: gene therapy approaches to sickle cell disease. Blood 127, 839-848.
6. Aygun, B., and Odame, I. (2012). A global perspective on sickle cell disease. Pediatr. Blood Cancer 59, 386-390.
7. Asadov, C., Alimirzoeva, Z., Mammadova, T., Aliyeva, G., Gafarova, S., and Mammadov, J. (2018). beta-Thalassemia intermedia: a comprehensive overview and novel approaches. Int. J. Hematol. 108, 5-21.
8. Taher, A. T., Musallam, K. M., and Cappellini, M. D. (2021). beta-Thalassemias. N. Engl. J. Med. 384, 727-743.
9. Thompson, A. A., Walters, M. C., Kwiatkowski, J., Rasko, J. E. J., Ribeil, J. A., Hongeng, S., Magrin, E., Schiller, G. J., Payen, E., Semeraro, M., et al. (2018). Gene Therapy in Patients with Transfusion-Dependent beta-Thalassemia. N. Engl. J. Med. 378, 1479-1493.
10. Magrin, E., Miccio, A., and Cavazzana, M. (2019). Lentiviral and genome-editing strategies for the treatment of beta-hemoglobinopathies. Blood 134, 1203-1213.
11. Charache, S., Terrin, M. L., Moore, R. D., Dover, G. J., Barton, F. B., Eckert, S. V., McMahon, R. P., and Bonds, D. R. (1995). Effect of hydroxyurea on the frequency of painful crises in sickle cell anemia. Investigators of the Multicenter Study of Hydroxyurea in Sickle Cell Anemia. N. Engl. J. Med. 332, 1317-1322.
12. Rodgers, G. P., Dover, G. J., Noguchi, C. T., Schechter, A. N., and Nienhuis, A. W. (1990). Hematologic responses of patients with sickle cell disease to treatment with hydroxyurea. N. Engl. J. Med. 322, 1037-1045.
13. Wong, T. E., Brandow, A. M., Lim, W., and Lottenberg, R. (2014). Update on the use of hydroxyurea therapy in sickle cell disease. Blood 124, 3850-3857.
14. Castro, O., Brambilla, D. J., Thorington, B., Reindorf, C. A., Scott, R. B., Gillette, P., Vera, J. C., and Levy, P. S. (1994). The acute chest syndrome in sickle cell disease: incidence and risk factors. The Cooperative Study of Sickle Cell Disease. Blood 84, 643-649.
15. Esrick, E. B., Lehmann, L. E., Biffi, A., Achebe, M., Brendel, C., Ciuculescu, M. F., Daley, H., MacKinnon, B., Morris, E., Federico, A., et al. (2021). Post-Transcriptional Genetic Silencing of BCL11A to Treat Sickle Cell Disease. N. Engl. J. Med. 384, 205-215.
16. Frangoul, H., Altshuler, D., Cappellini, M. D., Chen, Y. S., Domm, J., Eustace, B. K., Foell, J., de la Fuente, J., Grupp, S., Handgretinger, R., et al. (2021). CRISPR-Cas9 Gene Editing for Sickle Cell Disease and beta-Thalassemia. N. Engl. J. Med. 384, 252-260.
17. Platt, O. S., Brambilla, D. J., Rosse, W. F., Milner, P. F., Castro, O., Steinberg, M. H., and Klug, P. P. (1994). Mortality in sickle cell disease. Life expectancy and risk factors for early death. N. Engl. J. Med. 330, 1639-1644.
18. Platt, O. S., Thorington, B. D., Brambilla, D. J., Milner, P. F., Rosse, W. F., Vichinsky, E., and Kinney, T. R. (1991). Pain in sickle cell disease. Rates and risk factors. N. Engl. J. Med. 325, 11-16.
19. Menzel, S., Garner, C., Gut, I., Matsuda, F., Yamaguchi, M., Heath, S., Foglio, M., Zelenika, D., Boland, A., Rooks, H., et al. (2007). A QTL influencing F cell production maps to a gene encoding a zinc-finger protein on chromosome 2p15. Nat. Genet. 39, 1197-1199.
20. Uda, M., Galanello, R., Sanna, S., Lettre, G., Sankaran, V. G., Chen, W., Usala, G., Busonero, F., Maschio, A., Albai, G., et al. (2008). Genome-wide association study shows BCL11A associated with persistent fetal hemoglobin and amelioration of the phenotype of beta-thalassemia. Proc. Natl. Acad. Sci. USA 105, 1620-1625.
21. Sankaran, V. G., Menne, T. F., Xu, J., Akie, T. E., Lettre, G., Van Handel, B., Mikkola, H. K., Hirschhorn, J. N., Cantor, A. B., and Orkin, S. H. (2008). Human fetal hemoglobin expression is regulated by the developmental stage-specific repressor BCL11A. Science 322, 1839-1842.
22. Xu, J., Peng, C., Sankaran, V. G., Shao, Z., Esrick, E. B., Chong, B. G., Ippolito, G. C., Fujiwara, Y., Ebert, B. L., Tucker, P. W., et al. (2011). Correction of sickle cell disease in adult mice by interference with fetal hemoglobin silencing. Science 334, 993-996.
23. Smith, A. R., Schiller, G. J., Vercellotti, G. M., Kwiatkowski, J. L., Krishnamurti, L., Esrick, E. B., Williams, D. A., Miller, W. P., Woolfson, A., and Walters, M. C. (2019). Preliminary Results of a Phase 1/2 Clinical Study of Zinc Finger Nuclease-Mediated Editing of BCL11A in Autologous Hematopoietic Stem Cells for Transfusion-Dependent Beta Thalassemia. Blood 134, 3544-3544.
24. Demirci, S., Zeng, J., Wu, Y., Uchida, N., Shen, A. H., Pellin, D., Gamer, J., Yapundich, M., Drysdale, C., Bonanno, J., et al. (2020). BCL11A enhancer-edited hematopoietic stem cells persist in rhesus monkeys without toxicity. J. Clin. Invest. 130, 6677-6687.
25. Salinas Cisneros, G., and Thein, S. L. (2020). Recent Advances in the Treatment of Sickle Cell Disease. Front. Physiol. 11, 435.
26. Brendel, C., Guda, S., Renella, R., Bauer, D. E., Canver, M. C., Kim, Y. J., Heeney, M. M., Klatt, D., Fogel, J., Milsom, M. D., et al. (2016). Lineage-specific BCL11A knockdown circumvents toxicities and reverses sickle phenotype. J. Clin. Invest. 126, 3868-3878.
27. Guda, S., Brendel, C., Renella, R., Du, P., Bauer, D. E., Canver, M. C., Grenier, J. K., Grimson, A. W., Kamran, S. C., Thornton, J., et al. (2015). miRNA-embedded shRNAs for Lineage-specific BCL11 A Knockdown and Hemoglobin F Induction. Mol. Ther. 23, 1465-1474.
28. Brendel, C., Negre, O., Rothe, M., Guda, S., Parsons, G., Harris, C., McGuinness, M., Abriss, D., Tsytsykova, A., Klatt, D., et al. (2020). Preclinical Evaluation of a Novel Lentiviral Vector Driving Lineage-Specific BCL11A Knockdown for Sickle Cell Gene Therapy. Mol. Ther. Methods Clin. Dev. 17, 589-600.
29. Pires Lourenco, S., Jarocha, D., Ghiaccio, V., Guerra, A., Abdulmalik, O., La, P., Zezulin, A., Smith-Whitley, K., Kwiatkowski, J. L., Guzikowski, V., et al. (2021). Inclusion of a shRNA targeting BCL11A into a beta-globin expressing vector allows concurrent synthesis of curative adult and fetal hemoglobin. Haematologica.
30. Weber, L., Poletti, V., Magrin, E., Antoniani, C., Martin, S., Bayard, C., Sadek, H., Felix, T., Meneghini, V., Antoniou, M. N., et al. (2018). An Optimized Lentiviral Vector Efficiently Corrects the Human Sickle Cell Disease Phenotype. Mol. Ther. Methods Clin. Dev. 10, 268-280.
31. Fitzhugh, C. D., Cordes, S., Taylor, T., Coles, W., Roskom, K., Link, M., Hsieh, M. M., and Tisdale, J. F. (2017). At least 20% donor myeloid chimerism is necessary to reverse the sickle phenotype after allogeneic HSCT. Blood 130, 1946-1948.
32. Uchida, N., Ferrara, F., Drysdale, C. M., Yapundich, M., Gamer, J., Nassehi, T., DiNicola, J., Shibata, Y., Wielgosz, M., Kim, Y. S., et al. (2021). Sustained fetal hemoglobin induction in vivo is achieved by BCL11A interference and coexpressed truncated erythropoietin receptor. Sci. Transl. Med. 13.
33. Vinjamur, D. S., Yao, Q., Cole, M. A., McGuckin, C., Ren, C., Zeng, J., Hossain, M., Luk, K., Wolfe, S. A., Pinello, L., et al. (2021). ZNF410 represses fetal globin by singular control of CHD4. Nat. Genet. 53, 719-728.
34. Lan, X., Ren, R., Feng, R., Ly, L. C., Lan, Y., Zhang, Z., Aboreden, N., Qin, K., Horton, J. R., Grevet, J. D., et al. (2021). ZNF410 Uniquely Activates the NuRD Component CHD4 to Silence Fetal Hemoglobin Expression. Mol. Cell 81, 239-254 e238.
35. Kanter, J., Walters, M. C., Hsieh, M. M., Krishnamurti, L., Kwiatkowski, J., Kamble, R. T., von Kalle, C., Kuypers, F. A., Cavazzana, M., Leboulch, P., et al. (2016). Interim Results from a Phase 1/2 Clinical Study of Lentiglobin Gene Therapy for Severe Sickle Cell Disease. Blood 128, 1176-1176.
36. Poletti, V., Urbinati, F., Charrier, S., Corre, G., Hollis, R. P., Campo Fernandez, B., Martin, S., Rothe, M., Schambach, A., Kohn, D. B., et al. (2018). Pre-clinical Development of a Lentiviral Vector Expressing the Anti-sickling betaAS3 Globin for Gene Therapy for Sickle Cell Disease. Mol. Ther. Methods Clin. Dev. 11, 167-179.
37. Urbinati, F., Wherley, J., Geiger, S., Fernandez, B. C., Kaufman, M. L., Cooper, A., Romero, Z., Marchioni, F., Reeves, L., Read, E., et al. (2017). Preclinical studies for a phase 1 clinical trial of autologous hematopoietic stem cell gene therapy for sickle cell disease. Cytotherapy 19, 1096-1112.
38. Brunson, A., Keegan, T. H. M., Bang, H., Mahajan, A., Paulukonis, S., and Wun, T. (2017). Increased risk of leukemia among sickle cell disease patients in California. Blood 130, 1597-1599.
39. Morgan, R. A., Unti, M. J., Aleshe, B., Brown, D., Osborne, K. S., Koziol, C., Ayoub, P. G., Smith, O. B., O'Brien, R., Tam, C., et al. (2020). Improved Titer and Gene Transfer by Lentiviral Vectors Using Novel, Small beta-Globin Locus Control Region Elements. Mol. Ther. 28, 328-340.
40. Maier-Redelsperger, M., Noguchi, C. T., de Montalembert, M., Rodgers, G. P., Schechter, A. N., Gourbil, A., Blanchard, D., Jais, J. P., Ducrocq, R., and Peltier, J. Y. (1994). Variation in fetal hemoglobin parameters and predicted hemoglobin S polymerization in sickle cell children in the first two years of life: Parisian Prospective Study on Sickle Cell Disease. Blood 84, 3182-3188.
41. Poillon, W. N., Kim, B. C., Rodgers, G. P., Noguchi, C. T., and Schechter, A. N. (1993). Sparing effect of hemoglobin F and hemoglobin A2 on the polymerization of hemoglobin S at physiologic ligand saturations. Proc. Natl. Acad. Sci. USA 90, 5039-5043.
42. Brittenham, G. M., Schechter, A. N., and Noguchi, C. T. (1985). Hemoglobin S polymerization: primary determinant of the hemolytic and clinical severity of the sickling syndromes. Blood 65, 183-189.
43. Hebert N, E. E., Armant M, Brendel C, Ciuculescu M F, Audureau E, Higgins J M, Williams D A, Bartolucci, P (2021). Effects of BCL11A Shmir-Induced Post-Transcriptional Silencing on Distributions of HbF in Single-RBCs and Reticulocytes. Blood (ASH Annual Meeting Abstracts).
44. De Souza D C, E. E., Hebert N, Di Caprio G, Ciuculescu M F, Morris E, Armant M, Gonçalves B P, Schonbrun E, Brendel C, Wood D K, Bartolucci P, Williams D A, and Higgins J M, (2021). Effects of BCL11A shmiR-Induced Post-Transcriptional Silencing on Hemoglobin Polymer Inhibition in Single Red Blood Cells at Physiologic Oxygen Tension. Blood (ASH Annual Meeting Abstracts).
45. Steinberg, M. H., Chui, D. H., Dover, G. J., Sebastiani, P., and Alsultan, A. (2014). Fetal hemoglobin in sickle cell anemia: a glass half full? Blood 123, 481-485.
46. Khandros, E., Huang, P., Peslak, S. A., Sharma, M., Abdulmalik, O., Giardine, B. M., Zhang, Z., Keller, C. A., Hardison, R. C., and Blobel, G. A. (2020). Understanding heterogeneity of fetal hemoglobin induction through comparative analysis of F and A erythroblasts. Blood 135, 1957-1968.
47. Masuda, T., Wang, X., Maeda, M., Canver, M. C., Sher, F., Funnell, A. P., Fisher, C., Suciu, M., Martyn, G. E., Norton, L. J., et al. (2016). Transcription factors LRF and BCL11A independently repress expression of fetal hemoglobin. Science 351, 285-289.
48. Gudmundsdottir, B., Gudmundsson, K. O., Klarmann, K. D., Singh, S. K., Sun, L., Singh, S., Du, Y., Coppola, V., Stockwin, L., Nguyen, N., et al. (2018). POGZ Is Required for Silencing Mouse Embryonic beta-like Hemoglobin and Human Fetal Hemoglobin Expression. Cell Rep. 23, 3236-3248.
49. Bauer, D. E., Kamran, S. C., Lessard, S., Xu, J., Fujiwara, Y., Lin, C., Shao, Z., Canver, M. C., Smith, E. C., Pinello, L., et al. (2013). An erythroid enhancer of BCL11A subjectto genetic variation determines fetal hemoglobin level. Science 342, 253-257.
50. Metais, J. Y., Doerfler, P. A., Mayuranathan, T., Bauer, D. E., Fowler, S. C., Hsieh, M. M., Katta, V., Keriwala, S., Lazzarotto, C. R., Luk, K., et al. (2019). Genome editing of HBG1 and HBG2 to induce fetal hemoglobin. Blood Adv. 3, 3379-3392.
51. Ye, L., Wang, J., Tan, Y., Beyer, A. I., Xie, F., Muench, M. O., and Kan, Y. W. (2016). Genome editing using CRISPR-Cas9 to create the HPFH genotype in HSPCs: An approach for treating sickle cell disease and beta-thalassemia. Proc. Natl. Acad. Sci. USA 113, 10661-10665.
52. Ramadier, S., Chalumeau, A., Felix, T., Othman, N., Aknoun, S., Casini, A., Maule, G., Masson, C., De Cian, A., Frati, G., et al. (2021). Combination of lentiviral and genome editing technologies for the treatment of sickle cell disease. Mol. Ther. 10.1016/j.ymthe.2021.08.019.
53. Zuccaro, M. V., Xu, J., Mitchell, C., Marin, D., Zimmerman, R., Rana, B., Weinstein, E., King, R. T., Palmerola, K. L., Smith, M. E., et al. (2020). Allele-Specific Chromosome Removal after Cas9 Cleavage in Human Embryos. Cell 183, 1650-1664 e1615.
54. Song, Y., Liu, Z., Zhang, Y., Chen, M., Sui, T., Lai, L., and Li, Z. (2020). Large-Fragment Deletions Induced by Cas9 Cleavage while Not in the BEs System. Mol Ther Nucleic Acids 21, 523-526.
55. Enache, O. M., Rendo, V., Abdusamad, M., Lam, D., Davison, D., Pal, S., Currimjee, N., Hess, J., Pantel, S., Nag, A., et al. (2020). Cas9 activates the p53 pathway and selects for p53-inactivating mutations. Nat. Genet. 52, 662-668.
56. Demirci, S., Gudmundsdottir, B., Li, Q., Haro-Mora, J. J., Nassehi, T., Drysdale, C., Yapundich, M., Gamer, J., Seifuddin, F., Tisdale, J. F., et al. (2020). betaT87Q-Globin Gene Therapy Reduces Sickle Hemoglobin Production, Allowing for Ex Vivo Anti-sickling Activity in Human Erythroid Cells. Mol. Ther. Methods Clin. Dev. 17, 912-921.
57. Giarratana, M. C., Rouard, H., Dumont, A., Kiger, L., Safeukui, I., Le Pennec, P. Y., Francois, S., Trugnan, G., Peyrard, T., Marie, T., et al. (2011). Proof of principle for transfusion of in vitro-generated red blood cells. Blood 118, 5071-5079.

### Example 2: Exemplary Double shmiR Lentiviral (LV) Sequences

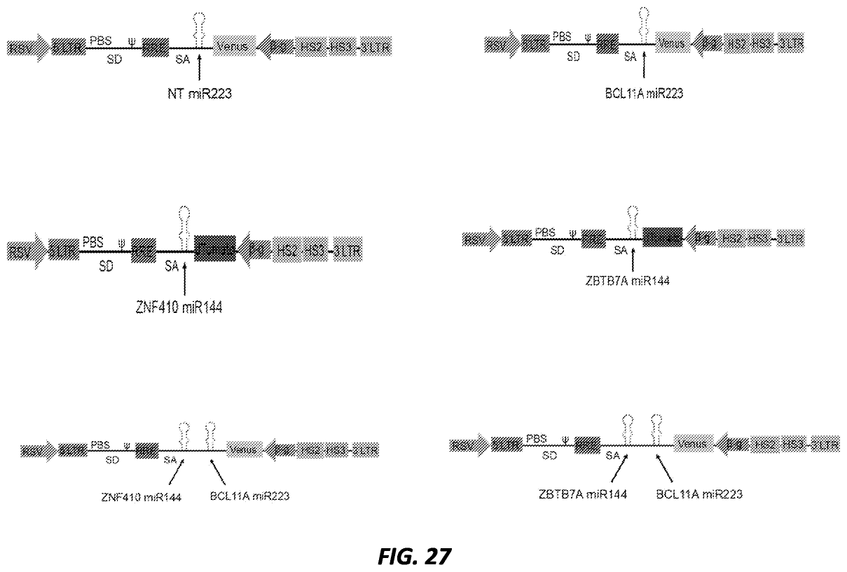

LV-LCR-NT
(SEQ ID NO: 23; see FIG. 27 for schematic)
TTAATGTAGTCTTATGCAATACTCTTGTAGTCTTGCAACATGGTAACGATGAGTTAGCAACA
TGCCTTACAAGGAGAGAAAAAGCACCGTGCATGCCGATTGGTGGAAGTAAGGTGGTACGATC
GTGCCTTATTAGGAAGGCAACAGACGGGTCTGACATGGATTGGACGAACCACTGAATTGCCG
CATTGCAGAGATATTGTATTTAAGTGCCTAGCTCGATACATAAACGGGTCTCTCTGGTTAGA
CCAGATCTGAGCCTGGGAGCTCTCTGGCTAACTAGGGAACCCACTGCTTAAGCCTCAATAAA
GCTTGCCTTGAGTGCTTCAAGTAGTGTGTGCCCGTCTGTTGTGTGACTCTGGTAACTAGAGA
TCCCTCAGACCCTTTTAGTCAGTGTGGAAAATCTCTAGCAGTGGCGCCCGAACAGGGACTTG
AAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGA
AGGAGAGAGATGGGTGCGAGAGCGTCAGTATTAAGCGGGGGAGAATTAGATCGCGATGGGAA
AAAATTCGGTTAAGGCCAGGGGGAAAGAAAAAATATAAATTAAAACATATAGTATGGGCAAG
CAGGGAGCTAGAACGATTCGCAGTTAATCCTGGCCTGTTAGAAACATCAGAAGGCTGTAGAC
AAATACTGGGACAGCTACAACCATCCCTTCAGACAGGATCAGAAGAACTTAGATCATTATAT
AATACAGTAGCAACCCTCTATTGTGTGCATCAAAGGATAGAGATAAAAGACACCAAGGAAGC
TTTAGACAAGATAGAGGAAGAGCAAAACAAAAGTAAGACCACCGCACAGCAAGCGGCCGCTG
ATCTTCAGACCTGGAGGAGGAGATATGAGGGACAATTGGAGAAGTGAATTATATAAATATAA
AGTAGTAAAAATTGAACCATTAGGAGTAGCACCCACCAAGGCAAAGAGAAGAGTGGTGCAGA
GAGAAAAAAGAGCAGTGGGAATAGGAGCTTTGTTCCTTGGGTTCTTGGGAGCAGCAGGAAGC
ACTATGGGCGCAGCGTCAATGACGCTGACGGTACAGGCCAGACAATTATTGTCTGGTATAGT
GCAGCAGCAGAACAATTTGCTGAGGGCTATTGAGGCGCAACAGCATCTGTTGCAACTCACAG
TCTGGGGCATCAAGCAGCTCCAGGCAAGAATCCTGGCTGTGGAAAGATACCTAAAGGATCAA
CAGCTCCTGGGGATTTGGGGTTGCTCTGGAAAACTCATTTGCACCACTGCTGTGCCTTGGAA
TGCTAGTTGGAGTAATAAATCTCTGGAACAGATTTGGAATCACACGACCTGGATGGAGTGGG
ACAGAGAAATTAACAATTACACAAGCTTAATACACTCCTTAATTGAAGAATCGCAAAACCAG
CAAGAAAAGAATGAACAAGAATTATTGGAATTAGATAAATGGGCAAGTTTGTGGAATTGGTT
TAACATAACAAATTGGCTGTGGTATATAAAATTATTCATAATGATAGTAGGAGGCTTGGTAG
GTTTAAGAATAGTTTTTGCTGTACTTTCTATAGTGAATAGAGTTAGGCAGGGATATTCACCA
TTATCGTTTCAGACCCACCTCCCAACCCCGAGGGGACCCGACAGGCCCGAAGGAATAGAAGA
AGAAGGTGGAGAGAGAGACAGAGACAGATCCATTCGATTAGTGAACGGATCTCGACGGTATC
GGTTAACTTTTAAAAGAAAAGGGGGGATTGGGGGGTACAGTGCAGGGGAAAGAATAGTAGAC
ATAATAGCAACAGACATACAAACTAAAGAATTACAAAAACAAATTACAAAATTCAAAATTTT
ATCGATAAGCTTGATGGCCGCGCTTAATGCGCCGCTACAGGGCGCGTGGGGATACCCCCTAG
AGCCCCAGCTGGTTCTTTCCGCCTCAGAAGCCATAGAGCCCACCGCATCCCCAGCATGCCTG
CTATTGTCTTCCCAATCCTCCCCCTTGCTGTCCTGCCCCACCCCACCCCCCAGAATAGAATG
ACACCTACTCAGACAATGCGATGCAATTTCCTCATTTTATTAGGAAAGGACAGTGGGAGTGG
CACCTTCCAGGGTCAAGGAAGGCACGGGGGAGGGGCAAACAACAGATGGCTGGCAGTCCATT
CGTCATATCCCATCTGCCCTGGCCTAGAGCTGGTAAGCATGTGCCGCACTCAACAAGATGAA
GAGCACCAACTCTACCACATGGAGTTGGTGCTCTTCATCTTGTTGAGCGTGGCACTGCAGGA
GGCCAGGCCAAGAGCTTCTGTGGGGAAGTGAGATCCCCCGGGGGAATTCGATATCAAGCTTA
TCGATACCGTCGACCTCGAGGGGGGGCCGCTTACTTGTACAGCTCGTCCATGCCGAGAGTGA
TCCCGGCGGCGGTCACGAACTCCAGCAGGACCATGTGATCGCGCTTCTCGTTGGGGTCTTTG
CTCAGGGCGGACTGGTAGCTCAGGTAGTGGTTGTCGGGCAGCAGCACGGGGCCGTCGCCGAT
GGGGGTGTTCTGCTGGTAGTGGTCGGCGAGCTGCACGCCGCCGTCCTCGATGTTGTGGCGGA
TCTTGAAGTTGGCCTTGATGCCGTTCTTCTGCTTGTCGGCGGTGATATAGACGTTGTGGCTG
TTGTAGTTGTACTCCAGCTTGTGCCCCAGGATGTTGCCGTCCTCCTTGAAGTCGATGCCCTT
CAGCTCGATGCGGTTCACCAGGGTGTCGCCCTCGAACTTCACCTCGGCGCGGGTCTTGTAGT
TGCCGTCGTCCTTGAAGAAGATGGTGCGCTCCTGGACGTAGCCTTCGGGCATGGCGGACTTG
AAGAAGTCGTGCTGCTTCATGTGGTCGGGGTAGCGGGCGAAGCACTGCAGGCCGTAGCCCAG
GGTGGTCACGAGGGTGGGCCAGGGCACGGGCAGCTTGCCGGTGGTGCAGATCAGCTTCAGGG
TCAGCTTGCCGTAGGTGGCATCGCCCTCGCCCTCGCCGGACACGCTGAACTTGTGGCCGTTT
ACGTCGCCGTCCAGCTCGACCAGGATGGGCACCACCCCGGTGAACAGCTCCTCGCCCTTGCT
CACCATGCCATCCACGTGATCGATGCTAGCTATGGTGTCTGTTTGAGGTTGCTAGTGAACAC
AGTTGTGTCAGAAGCAAATGTAAGCAATAGATGGCTCTGCCCTGACTTTTATGCCCAGCCCT
GGCTCCTGCCCTCCCTGCTCCTGGGAGTAGATTGGCCAACCCTAGGGTGTGGCTCCACAGGG
TGAGGTCTAAGTGATGACAGCCGTACCTGTCCTTGGCTCTTCTGGCACTGGCTTAGGAGTTG
GACTTCAAACCCTCAGCCCTCCCTCTAAGATATATCTCTTGGCCCCATACCATCAGTACAAA
TTGCTACTAAAAACATCCTCCTTTGCAAGTGTATTTACACGGTATCGATAAGCTTGAATTCC
TGCAGCCCCCTTTTGCCACCTAGCTGTCCAGGGGTGCCTTAAAATGGCAAACAAGGTTTGTT
TTCTTTTCCTGTTTTCATGCCTTCCTCTTCCATATCCTTGTTTCATATTAATACATGTGTAT
AGATCCTAAAAATCTATACACATGTATTAATAAAGCCTGATTCTGCCGCTTCTAGGTATAGA
GGCCACCTGCAAGATAAATATTTGATTCACAATAACTAATCATTCTATGGCAATTGATAACA
ACAAATATATATATATATATATATACGTATATGTGTATATATATATATATATATTCAGGAAA
TAATATATTCTAGAATATGTCACATTCTGTCTCAGGCATCCATTTTCTTTATGATGCCGTTT
GAGGTGGAGTTTTAGTCAGGTGGTCAGCTTCTCCTTTTTTTTGCCATCTGCCCTGTAAGCAT
CCTGCTGGGGACCCAGATAGGAGTCATCACTCTAGGCTGAGAACATCTGGGCACACACCCTA
AGCCTCAGCATGACTCATCATGACTCAGCATTGCTGTGCTTGAGCCAGAAGGTTTGCTTAGA
AGGTTACACAGAACCAGAAGGCGGGGGTGGGGCACTGACCCCGACAGGGGCCTGGCCAGAAC
TGCTCATGCTTGGACTATGGGAGGTCACTAATGGAGACACACAGAAATGTAACAGGAACTAA
GGAAAAACTGAAGCTTATTTAATCAGAGATGAGGATGCTGGAAGGGATAGAGGGAGCTGAGC
TTGTAAAAAGTATAGTAATCATTCAGCAAATGGTTTTGAAGCACCTGCTGGATGCTAAACAC
TATTTTCAGTGCTTGAATCATAAATAAGAATAAAACATGTATCTTATTCCCCACAAGAGTCC
AAGTAAAAAATAACAGTTAATTATAATGTGCTCTGTCCCCCAGGCTGGAGTGCAGTGGCACG
ATCTCAGCTCACTGCAACCTCCGCCTCCCGGGGGGTTCAAGCAATTCTCCTGCCTCAGCCAC
CCTAATAGCTGGGATTACAGGTGCACACCACCATGCCAGGCTAATTTTTGTACTTTTTGTAG
AGGTTTTGTACTTTTTGTAGAGGCAGGGTATCACCATGTTGTCCAAGATGGTCTTGAACTCC
TGAGCTCCAAGCAGTCCACCCACCTCAGCCTCCCAAAGTGCTGGGATTACAGGTGTGAGACA
CCATGCCCAGATTTTCCATATTTAATAGAGGTATTTATGGGATGGGGGAAAAGAATGTTTCT
CTCACTGTGGATTATTTTAGAGAGTGGAGAATGGTCAAGATTTTTTTAAAAATTAAGAAAAC
ATAAGTTGGACCTTGAGAAATGAAAATTTATTTTTTTGTTGGAGGATACCCATTCTCTATCT
CCCATCAGGGCAAGCTGTAAGGAACTGGCTAAGACACAGTGAGACAGAGTGACTTAGTCTTA
GAGGCCCCACTGGTACGACGGTCACCAAGCTTTCATTAAAAAAAGTCTAACCAGCTGCATTC
GACTTTGACTGCAGCAGCTGGTTAGAAGGTTCTACTGGAGGAGGGTCCCAGCCCATTGCTAA
ATTAACATCAGGCTCTGAGACTGGCAGTATATCTCTAACAGTGGTTGATGCTATCTTCTGGA
ACTTGCCTGCTACATTGAGACCACTGACCCATACATAGGAAGCCCATAGCTCTGTCCTGAAC
TGTTAGGCCACTGGTCCAGAGAGTGTGCATCTCCTTTGATCCTCATAATAACCCTATGAGAT
AGACACAATTATTACTCTTACTTTATAGATGATGATCCTGAAAACATAGGAGTCAAGGCACT
TGCCCCTAGCTGGGGGTATAGGGGAGCAGTCCCATGTAGTAGTAGAATGAAAAATGCTGCTA
TGCTGTGCCTCCCCCACCTTTCCCATGTCTGCCCTCTACTCATGGTCTATCTCTCCTGGCTC
CTGGGAGTCATGGACTCCACCCAGCACCACCAACCTGACCTAACCACCTATCTGAGCCTGCC
AGCCTATAACCCATCTGGGCCCTGATAGCTGGTGGCCAGCCCTGACCCCACCCCACCCTCCC
TGGAACCTCTGATAGACACATCTGGCACACCAGCTCGCAAAGTCACCGTGAGGGTCTTGTGT
TTGCTGAGTCAAAATTCCTTGAAATCCAAGTCCTTAGAGACTCCTGCTCCCAAATTTACAGT
CATAGACTTCTTCATGGCTGTCTCCTTTATCCACAGAATGATTCCTTTGCTTCATTGCCCCA
TCCATCTGATCCTCCTCATCAGTGCAGCACAGGGCCCATGAGCAGTAGCTGCAGAGTCTCAC
ATAGGTCTGGCACTGCCTCTGACATGTCCGACCTTAGGCAAATGCTTGACTCTTCTGAGCTC
AGTCTTGTCATGGCAAAATAAAGATAATAATAGTGTTTTTTTATGGAGTTAGCGTGAGGATG
GAAAACAATAGCAAAATTGATTAGACTATAAAAGGTCTCAACAAATAGTAGTAGATTTTATC
ATCCATTAATCCTTCCCTCTCCTCTCTTACTCATCCCATCACGTATGCCTCTTAATTTTCCC
TTACCTATAATAAGAGTTATTCCTCTTATTATATTCTTCTTATAGTGATTCTGGATATTAAA
GTGGGAATGAGGGGCAGGCCACTAACGAAGAAGATGTTTCTCAAAGAAGCGGGGGATCCACT
AGTTCTAGAGCGGCCGTACTCGAGGTTTAAACAGTACCTTTAAGACCAATGACTTACAAGGC
AGCTGTAGATCTTAGCCACTTTTTAAAAGAAAAGGGGGGACTGGAAGGGCTAATTCACTCCC
AACGAAGACAAGATCTGCTTTTTGCTTGTACTGGGTCTCTCTGGTTAGACCAGATCTGAGCC
TGGGAGCTCTCTGGCTAACTAGGGAACCCACTGCTTAAGCCTCAATAAAGCTTGCCTTGAGT
GCTTCAAGTAGTGTGTGCCCGTCTGTTGTGTGACTCTGGTAACTAGAGATCCCTCAGACCCT
TTTAGTCAGTGTGGAAAATCTCTAGCA
Underlined text: RSV
Double underlined text: 5′ LTR
Underlined, italicized text: RRE
Bold, underlined text: NT shmiR in mir223
Bold text: Venus
Italicized text: Beta-globin promoter and LCR
LV-LCR-BCL11A
(SEQ ID NO: 24; see FIG. 27 for schematic)
TTAATGTAGTCTTATGCAATACTCTTGTAGTCTTGCAACATGGTAACGATGAGTTAGCAACA
TGCCTTACAAGGAGAGAAAAAGCACCGTGCATGCCGATTGGTGGAAGTAAGGTGGTACGATC
GTGCCTTATTAGGAAGGCAACAGACGGGTCTGACATGGATTGGACGAACCACTGAATTGCCG
CATTGCAGAGATATTGTATTTAAGTGCCTAGCTCGATACATAAACGGGTCTCTCTGGTTAGA
CCAGATCTGAGCCTGGGAGCTCTCTGGCTAACTAGGGAACCCACTGCTTAAGCCTCAATAAA
GCTTGCCTTGAGTGCTTCAAGTAGTGTGTGCCCGTCTGTTGTGTGACTCTGGTAACTAGAGA
TCCCTCAGACCCTTTTAGTCAGTGTGGAAAATCTCTAGCAGTGGCGCCCGAACAGGGACTTG
AAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGA
AGGAGAGAGATGGGTGCGAGAGCGTCAGTATTAAGCGGGGGAGAATTAGATCGCGATGGGAA
AAAATTCGGTTAAGGCCAGGGGGAAAGAAAAAATATAAATTAAAACATATAGTATGGGCAAG
CAGGGAGCTAGAACGATTCGCAGTTAATCCTGGCCTGTTAGAAACATCAGAAGGCTGTAGAC
AAATACTGGGACAGCTACAACCATCCCTTCAGACAGGATCAGAAGAACTTAGATCATTATAT
AATACAGTAGCAACCCTCTATTGTGTGCATCAAAGGATAGAGATAAAAGACACCAAGGAAGC
TTTAGACAAGATAGAGGAAGAGCAAAACAAAAGTAAGACCACCGCACAGCAAGCGGCCGCTG
ATCTTCAGACCTGGAGGAGGAGATATGAGGGACAATTGGAGAAGTGAATTATATAAATATAA
AGTAGTAAAAATTGAACCATTAGGAGTAGCACCCACCAAGGCAAAGAGAAGAGTGGTGCAGA
GAGAAAAAAGAGCAGTGGGAATAGGAGCTTTGTTCCTTGGGTTCTTGGGAGCAGCAGGAAGC
ACTATGGGCGCAGCGTCAATGACGCTGACGGTACAGGCCAGACAATTATTGTCTGGTATAGT
GCAGCAGCAGAACAATTTGCTGAGGGCTATTGAGGCGCAACAGCATCTGTTGCAACTCACAG
TCTGGGGCATCAAGCAGCTCCAGGCAAGAATCCTGGCTGTGGAAAGATACCTAAAGGATCAA
CAGCTCCTGGGGATTTGGGGTTGCTCTGGAAAACTCATTTGCACCACTGCTGTGCCTTGGAA
TGCTAGTTGGAGTAATAAATCTCTGGAACAGATTTGGAATCACACGACCTGGATGGAGTGGG
ACAGAGAAATTAACAATTACACAAGCTTAATACACTCCTTAATTGAAGAATCGCAAAACCAG
CAAGAAAAGAATGAACAAGAATTATTGGAATTAGATAAATGGGCAAGTTTGTGGAATTGGTT
TAACATAACAAATTGGCTGTGGTATATAAAATTATTCATAATGATAGTAGGAGGCTTGGTAG
GTTTAAGAATAGTTTTTGCTGTACTTTCTATAGTGAATAGAGTTAGGCAGGGATATTCACCA
TTATCGTTTCAGACCCACCTCCCAACCCCGAGGGGACCCGACAGGCCCGAAGGAATAGAAGA
AGAAGGTGGAGAGAGAGACAGAGACAGATCCATTCGATTAGTGAACGGATCTCGACGGTATC
GGTTAACTTTTAAAAGAAAAGGGGGGATTGGGGGGTACAGTGCAGGGGAAAGAATAGTAGAC
ATAATAGCAACAGACATACAAACTAAAGAATTACAAAAACAAATTACAAAATTCAAAATTTT
ATCGATAAGCTTGATGGCCGCGCTTAATGCGCCGCTACAGGGCGCGTGGGGATACCCCCTAG
AGCCCCAGCTGGTTCTTTCCGCCTCAGAAGCCATAGAGCCCACCGCATCCCCAGCATGCCTG
CTATTGTCTTCCCAATCCTCCCCCTTGCTGTCCTGCCCCACCCCACCCCCCAGAATAGAATG
ACACCTACTCAGACAATGCGATGCAATTTCCTCATTTTATTAGGAAAGGACAGTGGGAGTGG
CACCTTCCAGGGTCAAGGAAGGCACGGGGGAGGGGCAAACAACAGATGGCTGGCAACTAGAA
GGCACAGTCGAGGCTGATCAGCGAGCTCTAGCATTTAGGTGACACTATAGAATAGGGCGCTC
TAGAACTAGTGGATCCCCCGGGGGAATTCGCCCTTACGCGTCGACGTCGGTGAGCATCCTTG
TATCCAGCTGGCAGTCCATTCGTCATATCCCATCTGCCCTGGCCTAGAGCTGGTAAGCATGT
GCCGCACTGCGCGATCGAGTGTTGAATAACTCTACCACATGGAGTTATTCAACACTCGATCG
CGCAGCGTGGCACTGCAGGAGGCCAGGCCAAGAGCTTCTGTGGGGAAGTGAGATCCCCCGGG
GGAATTCGATATCAAGCTTATCGATACCGTCGACCTCGAGGGGGGGCCGCTTACTTGTACAG
CTCGTCCATGCCGAGAGTGATCCCGGCGGCGGTCACGAACTCCAGCAGGACCATGTGATCGC
GCTTCTCGTTGGGGTCTTTGCTCAGGGCGGACTGGTAGCTCAGGTAGTGGTTGTCGGGCAGC
AGCACGGGGCCGTCGCCGATGGGGGTGTTCTGCTGGTAGTGGTCGGCGAGCTGCACGCCGCC
GTCCTCGATGTTGTGGCGGATCTTGAAGTTGGCCTTGATGCCGTTCTTCTGCTTGTCGGCGG
TGATATAGACGTTGTGGCTGTTGTAGTTGTACTCCAGCTTGTGCCCCAGGATGTTGCCGTCC
TCCTTGAAGTCGATGCCCTTCAGCTCGATGCGGTTCACCAGGGTGTCGCCCTCGAACTTCAC
CTCGGCGCGGGTCTTGTAGTTGCCGTCGTCCTTGAAGAAGATGGTGCGCTCCTGGACGTAGC
CTTCGGGCATGGCGGACTTGAAGAAGTCGTGCTGCTTCATGTGGTCGGGGTAGCGGGCGAAG
CACTGCAGGCCGTAGCCCAGGGTGGTCACGAGGGTGGGCCAGGGCACGGGCAGCTTGCCGGT
GGTGCAGATCAGCTTCAGGGTCAGCTTGCCGTAGGTGGCATCGCCCTCGCCCTCGCCGGACA
CGCTGAACTTGTGGCCGTTTACGTCGCCGTCCAGCTCGACCAGGATGGGCACCACCCCGGTG
AACAGCTCCTCGCCCTTGCTCACCATGCCATCCACGTGATCGATGCTAGCTATGGTGTCTGT
TTGAGGTTGCTAGTGAACACAGTTGTGTCAGAAGCAAATGTAAGCAATAGATGGCTCTGCCC
TGACTTTTATGCCCAGCCCTGGCTCCTGCCCTCCCTGCTCCTGGGAGTAGATTGGCCAACCC
TAGGGTGTGGCTCCACAGGGTGAGGTCTAAGTGATGACAGCCGTACCTGTCCTTGGCTCTTC
TGGCACTGGCTTAGGAGTTGGACTTCAAACCCTCAGCCCTCCCTCTAAGATATATCTCTTGG
CCCCATACCATCAGTACAAATTGCTACTAAAAACATCCTCCTTTGCAAGTGTATTTACACGG
TATCGATAAGCTTGAATTCCTGCAGCCCCCTTTTGCCACCTAGCTGTCCAGGGGTGCCTTAA
AATGGCAAACAAGGTTTGTTTTCTTTTCCTGTTTTCATGCCTTCCTCTTCCATATCCTTGTT
TCATATTAATACATGTGTATAGATCCTAAAAATCTATACACATGTATTAATAAAGCCTGATT
CTGCCGCTTCTAGGTATAGAGGCCACCTGCAAGATAAATATTTGATTCACAATAACTAATCA
TTCTATGGCAATTGATAACAACAAATATATATATATATATATATACGTATATGTGTATATAT
ATATATATATATTCAGGAAATAATATATTCTAGAATATGTCACATTCTGTCTCAGGCATCCA
TTTTCTTTATGATGCCGTTTGAGGTGGAGTTTTAGTCAGGTGGTCAGCTTCTCCTTTTTTTT
GCCATCTGCCCTGTAAGCATCCTGCTGGGGACCCAGATAGGAGTCATCACTCTAGGCTGAGA
ACATCTGGGCACACACCCTAAGCCTCAGCATGACTCATCATGACTCAGCATTGCTGTGCTTG
AGCCAGAAGGTTTGCTTAGAAGGTTACACAGAACCAGAAGGCGGGGGTGGGGCACTGACCCC
GACAGGGGCCTGGCCAGAACTGCTCATGCTTGGACTATGGGAGGTCACTAATGGAGACACAC
AGAAATGTAACAGGAACTAAGGAAAAACTGAAGCTTATTTAATCAGAGATGAGGATGCTGGA
AGGGATAGAGGGAGCTGAGCTTGTAAAAAGTATAGTAATCATTCAGCAAATGGTTTTGAAGC
ACCTGCTGGATGCTAAACACTATTTTCAGTGCTTGAATCATAAATAAGAATAAAACATGTAT
CTTATTCCCCACAAGAGTCCAAGTAAAAAATAACAGTTAATTATAATGTGCTCTGTCCCCCA
GGCTGGAGTGCAGTGGCACGATCTCAGCTCACTGCAACCTCCGCCTCCCGGGGGGTTCAAGC
AATTCTCCTGCCTCAGCCACCCTAATAGCTGGGATTACAGGTGCACACCACCATGCCAGGCT
AATTTTTGTACTTTTTGTAGAGGTTTTGTACTTTTTGTAGAGGCAGGGTATCACCATGTTGT
CCAAGATGGTCTTGAACTCCTGAGCTCCAAGCAGTCCACCCACCTCAGCCTCCCAAAGTGCT
GGGATTACAGGTGTGAGACACCATGCCCAGATTTTCCATATTTAATAGAGGTATTTATGGGA
TGGGGGAAAAGAATGTTTCTCTCACTGTGGATTATTTTAGAGAGTGGAGAATGGTCAAGATT
TTTTTAAAAATTAAGAAAACATAAGTTGGACCTTGAGAAATGAAAATTTATTTTTTTGTTGG
AGGATACCCATTCTCTATCTCCCATCAGGGCAAGCTGTAAGGAACTGGCTAAGACACAGTGA
GACAGAGTGACTTAGTCTTAGAGGCCCCACTGGTACGACGGTCACCAAGCTTTCATTAAAAA
AAGTCTAACCAGCTGCATTCGACTTTGACTGCAGCAGCTGGTTAGAAGGTTCTACTGGAGGA
GGGTCCCAGCCCATTGCTAAATTAACATCAGGCTCTGAGACTGGCAGTATATCTCTAACAGT
GGTTGATGCTATCTTCTGGAACTTGCCTGCTACATTGAGACCACTGACCCATACATAGGAAG
CCCATAGCTCTGTCCTGAACTGTTAGGCCACTGGTCCAGAGAGTGTGCATCTCCTTTGATCC
TCATAATAACCCTATGAGATAGACACAATTATTACTCTTACTTTATAGATGATGATCCTGAA
AACATAGGAGTCAAGGCACTTGCCCCTAGCTGGGGGTATAGGGGAGCAGTCCCATGTAGTAG
TAGAATGAAAAATGCTGCTATGCTGTGCCTCCCCCACCTTTCCCATGTCTGCCCTCTACTCA
TGGTCTATCTCTCCTGGCTCCTGGGAGTCATGGACTCCACCCAGCACCACCAACCTGACCTA
ACCACCTATCTGAGCCTGCCAGCCTATAACCCATCTGGGCCCTGATAGCTGGTGGCCAGCCC
TGACCCCACCCCACCCTCCCTGGAACCTCTGATAGACACATCTGGCACACCAGCTCGCAAAG
TCACCGTGAGGGTCTTGTGTTTGCTGAGTCAAAATTCCTTGAAATCCAAGTCCTTAGAGACT
CCTGCTCCCAAATTTACAGTCATAGACTTCTTCATGGCTGTCTCCTTTATCCACAGAATGAT
TCCTTTGCTTCATTGCCCCATCCATCTGATCCTCCTCATCAGTGCAGCACAGGGCCCATGAG
CAGTAGCTGCAGAGTCTCACATAGGTCTGGCACTGCCTCTGACATGTCCGACCTTAGGCAAA
TGCTTGACTCTTCTGAGCTCAGTCTTGTCATGGCAAAATAAAGATAATAATAGTGTTTTTTT
ATGGAGTTAGCGTGAGGATGGAAAACAATAGCAAAATTGATTAGACTATAAAAGGTCTCAAC
AAATAGTAGTAGATTTTATCATCCATTAATCCTTCCCTCTCCTCTCTTACTCATCCCATCAC
GTATGCCTCTTAATTTTCCCTTACCTATAATAAGAGTTATTCCTCTTATTATATTCTTCTTA
TAGTGATTCTGGATATTAAAGTGGGAATGAGGGGCAGGCCACTAACGAAGAAGATGTTTCTC
AAAGAAGCGGGGGATCCACTAGTTCTAGAGCGGCCGTACTCGAGGTTTAAACAGTACCTTTA
AGACCAATGACTTACAAGGCAGCTGTAGATCTTAGCCACTTTTTAAAAGAAAAGGGGGGACT
GGAAGGGCTAATTCACTCCCAACGAAGACAAGATCTGCTTTTTGCTTGTACTGGGTCTCTCT
GGTTAGACCAGATCTGAGCCTGGGAGCTCTCTGGCTAACTAGGGAACCCACTGCTTAAGCCT
CAATAAAGCTTGCCTTGAGTGCTTCAAGTAGTGTGTGCCCGTCTGTTGTGTGACTCTGGTAA
CTAGAGATCCCTCAGACCCTTTTAGTCAGTGTGGAAAATCTCTAGCAG
Underlined text: RSV
Double underlined text: 5' LTR
Underlined, italicized text: RRE
Bold, underlined text: BCL11A shmiR in mir223
Bold text: Venus
Italicized text: Beta-globin promoter and LCR
(SEQ ID NO: 25; see FIG. 27 for schematic)
TTAATGTAGTCTTATGCAATACTCTTGTAGTCTTGCAACATGGTAACGATGAGTTAGCAACA
TGCCTTACAAGGAGAGAAAAAGCACCGTGCATGCCGATTGGTGGAAGTAAGGTGGTACGATC
GTGCCTTATTAGGAAGGCAACAGACGGGTCTGACATGGATTGGACGAACCACTGAATTGCCG
CATTGCAGAGATATTGTATTTAAGTGCCTAGCTCGATACATAAACGGGTCTCTCTGGTTAGA
CCAGATCTGAGCCTGGGAGCTCTCTGGCTAACTAGGGAACCCACTGCTTAAGCCTCAATAAA
GCTTGCCTTGAGTGCTTCAAGTAGTGTGTGCCCGTCTGTTGTGTGACTCTGGTAACTAGAGA
TCCCTCAGACCCTTTTAGTCAGTGTGGAAAATCTCTAGCAGTGGCGCCCGAACAGGGACTTG
AAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGA
AGGAGAGAGATGGGTGCGAGAGCGTCAGTATTAAGCGGGGGAGAATTAGATCGCGATGGGAA
AAAATTCGGTTAAGGCCAGGGGGAAAGAAAAAATATAAATTAAAACATATAGTATGGGCAAG
CAGGGAGCTAGAACGATTCGCAGTTAATCCTGGCCTGTTAGAAACATCAGAAGGCTGTAGAC
AAATACTGGGACAGCTACAACCATCCCTTCAGACAGGATCAGAAGAACTTAGATCATTATAT
AATACAGTAGCAACCCTCTATTGTGTGCATCAAAGGATAGAGATAAAAGACACCAAGGAAGC
TTTAGACAAGATAGAGGAAGAGCAAAACAAAAGTAAGACCACCGCACAGCAAGCGGCCGCTG
ATCTTCAGACCTGGAGGAGGAGATATGAGGGACAATTGGAGAAGTGAATTATATAAATATAA
AGTAGTAAAAATTGAACCATTAGGAGTAGCACCCACCAAGGCAAAGAGAAGAGTGGTGCAGA
GAGAAAAAAGAGCAGTGGGAATAGGAGCTTTGTTCCTTGGGTTCTTGGGAGCAGCAGGAAGC
ACTATGGGCGCAGCGTCAATGACGCTGACGGTACAGGCCAGACAATTATTGTCTGGTATAGT
GCAGCAGCAGAACAATTTGCTGAGGGCTATTGAGGCGCAACAGCATCTGTTGCAACTCACAG
TCTGGGGCATCAAGCAGCTCCAGGCAAGAATCCTGGCTGTGGAAAGATACCTAAAGGATCAA
CAGCTCCTGGGGATTTGGGGTTGCTCTGGAAAACTCATTTGCACCACTGCTGTGCCTTGGAA
TGCTAGTTGGAGTAATAAATCTCTGGAACAGATTTGGAATCACACGACCTGGATGGAGTGGG
ACAGAGAAATTAACAATTACACAAGCTTAATACACTCCTTAATTGAAGAATCGCAAAACCAG
CAAGAAAAGAATGAACAAGAATTATTGGAATTAGATAAATGGGCAAGTTTGTGGAATTGGTT
TAACATAACAAATTGGCTGTGGTATATAAAATTATTCATAATGATAGTAGGAGGCTTGGTAG
GTTTAAGAATAGTTTTTGCTGTACTTTCTATAGTGAATAGAGTTAGGCAGGGATATTCACCA
TTATCGTTTCAGACCCACCTCCCAACCCCGAGGGGACCCGACAGGCCCGAAGGAATAGAAGA
AGAAGGTGGAGAGAGAGACAGAGACAGATCCATTCGATTAGTGAACGGATCTCGACGGTATC
GGTTAACTTTTAAAAGAAAAGGGGGGATTGGGGGGTACAGTGCAGGGGAAAGAATAGTAGAC
ATAATAGCAACAGACATACAAACTAAAGAATTACAAAAACAAATTACAAAATTCAAAATTTT
ATCGATAAGCTTGATGGCCGCGCTTAATGCGCCGCTACAGGGCGCGTGGGGATACCCCCTAG
AGCCCCAGCTGGTTCTTTCCGCCTCAGAAGCCATAGAGCCCACCGCATCCCCAGCATGCCTG
CTATTGTCTTCCCAATCCTCCCCCTTGCTGTCCTGCCCCACCCCACCCCCCAGAATAGAATG
ACACCTACTCAGACAATGCGATGCAATTTCCTCATTTTATTAGGAAAGGACAGTGGGAGTGG
CACCTTCCAGGGTCAAGGAAGGCACGGGGGAGGGGCAAACAACAGATGGCTGGCAACTAGAA
GGCACAGTCGAGGCTGATCAGCGAGCTCTAGCATTTAGGTGACACTATAGAATAGGGCGCTC
TAGAACTAGTGGATCCCCCGGGGGAATTCGCCCTTACGCGTATCTCTCCTGTCCTCCTTGTC
AGGCTCCAGAGCTGGGGGTGCCCGGACTGCTGAGCACTTAGTGTTTGTAGTGTCTCATCGCA
AACTTACAAACACTAAGTGCTCAGCCAGCCAGGGCCCCACTGGGGGCACAGGAAGCATGGCT
TGAAAAGCGACCTCGAGGGGGGGCCGCTTACTTGTACAGCTCGTCCATGCCGTACAGGAACA
GGTGGTGGCGGCCCTCGGAGCGCTCGTACTGTTCCACGATGGTGTAGTCCTCGTTGTGGGAG
GTGATGTCCAGCTTGGTGTCCACGTAGTAGTAGCCGGGCAGTTGCACGGGCTTCTTGGCCAT
GTAGATGGTCTTGAACTCCACCAGGTAGTGGCCGCCGTCCTTCAGCTTCAGGGCCTGGTGGA
TCTCGCCCTTCAGCACGCCGTCGCGGGGGTACAGGCGCTCGGTGGAGGCCTCCCAGCCCATG
GTCTTCTTCTGCATTACGGGGCCGTCGGGGGGGAAGTTGGTGCCGCGCATCTTCACCTTGTA
GATCAGCGTGCCGTCCTGCAGGGAGGAGTCCTGGGTCACGGTCACCAGACCGCCGTCCTCGA
AGTTCATCACGCGCTCCCACTTGAAGCCCTCGGGGAAGGACAGCTTCTTGTAATCGGGGATG
TCGGCGGGGTGCTTCACGTACGCCTTGGAGCCGTACATGAACTGGGGGGACAGGATGTCCCA
GGCGAAGGGCAGGGGGCCGCCCTTGGTCACCTTCAGCTTGGCGGTCTGGGTGCCCTCGTAGG
GGCGGCCCTCGCCCTCGCCCTCGATCTCGAACTCGTGGCCGTTCATGGAGCCCTCCATGCGC
ACCTTGAAGCGCATGAACTCTTTGATGACCTCCTCGCCCTTGCTCACCATGCCATCCACGTG
ATCGATGCTAGCTATGGTGTCTGTTTGAGGTTGCTAGTGAACACAGTTGTGTCAGAAGCAAA
TGTAAGCAATAGATGGCTCTGCCCTGACTTTTATGCCCAGCCCTGGCTCCTGCCCTCCCTGC
TCCTGGGAGTAGATTGGCCAACCCTAGGGTGTGGCTCCACAGGGTGAGGTCTAAGTGATGAC
AGCCGTACCTGTCCTTGGCTCTTCTGGCACTGGCTTAGGAGTTGGACTTCAAACCCTCAGCC
CTCCCTCTAAGATATATCTCTTGGCCCCATACCATCAGTACAAATTGCTACTAAAAACATCC
TCCTTTGCAAGTGTATTTACACGGTATCGATAAGCTTGAATTCCTGCAGCCCCCTTTTGCCA
CCTAGCTGTCCAGGGGTGCCTTAAAATGGCAAACAAGGTTTGTTTTCTTTTCCTGTTTTCAT
GCCTTCCTCTTCCATATCCTTGTTTCATATTAATACATGTGTATAGATCCTAAAAATCTATA
CACATGTATTAATAAAGCCTGATTCTGCCGCTTCTAGGTATAGAGGCCACCTGCAAGATAAA
TATTTGATTCACAATAACTAATCATTCTATGGCAATTGATAACAACAAATATATATATATAT
ATATATACGTATATGTGTATATATATATATATATATTCAGGAAATAATATATTCTAGAATAT
GTCACATTCTGTCTCAGGCATCCATTTTCTTTATGATGCCGTTTGAGGTGGAGTTTTAGTCA
GGTGGTCAGCTTCTCCTTTTTTTTGCCATCTGCCCTGTAAGCATCCTGCTGGGGACCCAGAT
AGGAGTCATCACTCTAGGCTGAGAACATCTGGGCACACACCCTAAGCCTCAGCATGACTCAT
CATGACTCAGCATTGCTGTGCTTGAGCCAGAAGGTTTGCTTAGAAGGTTACACAGAACCAGA
AGGCGGGGGTGGGGCACTGACCCCGACAGGGGCCTGGCCAGAACTGCTCATGCTTGGACTAT
GGGAGGTCACTAATGGAGACACACAGAAATGTAACAGGAACTAAGGAAAAACTGAAGCTTAT
TTAATCAGAGATGAGGATGCTGGAAGGGATAGAGGGAGCTGAGCTTGTAAAAAGTATAGTAA
TCATTCAGCAAATGGTTTTGAAGCACCTGCTGGATGCTAAACACTATTTTCAGTGCTTGAAT
CATAAATAAGAATAAAACATGTATCTTATTCCCCACAAGAGTCCAAGTAAAAAATAACAGTT
AATTATAATGTGCTCTGTCCCCCAGGCTGGAGTGCAGTGGCACGATCTCAGCTCACTGCAAC
CTCCGCCTCCCGGGGGGTTCAAGCAATTCTCCTGCCTCAGCCACCCTAATAGCTGGGATTAC
AGGTGCACACCACCATGCCAGGCTAATTTTTGTACTTTTTGTAGAGGTTTTGTACTTTTTGT
AGAGGCAGGGTATCACCATGTTGTCCAAGATGGTCTTGAACTCCTGAGCTCCAAGCAGTCCA
CCCACCTCAGCCTCCCAAAGTGCTGGGATTACAGGTGTGAGACACCATGCCCAGATTTTCCA
TATTTAATAGAGGTATTTATGGGATGGGGGAAAAGAATGTTTCTCTCACTGTGGATTATTTT
AGAGAGTGGAGAATGGTCAAGATTTTTTTAAAAATTAAGAAAACATAAGTTGGACCTTGAGA
AATGAAAATTTATTTTTTTGTTGGAGGATACCCATTCTCTATCTCCCATCAGGGCAAGCTGT
AAGGAACTGGCTAAGACACAGTGAGACAGAGTGACTTAGTCTTAGAGGCCCCACTGGTACGA
CGGTCACCAAGCTTTCATTAAAAAAAGTCTAACCAGCTGCATTCGACTTTGACTGCAGCAGC
TGGTTAGAAGGTTCTACTGGAGGAGGGTCCCAGCCCATTGCTAAATTAACATCAGGCTCTGA
GACTGGCAGTATATCTCTAACAGTGGTTGATGCTATCTTCTGGAACTTGCCTGCTACATTGA
GACCACTGACCCATACATAGGAAGCCCATAGCTCTGTCCTGAACTGTTAGGCCACTGGTCCA
GAGAGTGTGCATCTCCTTTGATCCTCATAATAACCCTATGAGATAGACACAATTATTACTCT
TACTTTATAGATGATGATCCTGAAAACATAGGAGTCAAGGCACTTGCCCCTAGCTGGGGGTA
TAGGGGAGCAGTCCCATGTAGTAGTAGAATGAAAAATGCTGCTATGCTGTGCCTCCCCCACC
TTTCCCATGTCTGCCCTCTACTCATGGTCTATCTCTCCTGGCTCCTGGGAGTCATGGACTCC
ACCCAGCACCACCAACCTGACCTAACCACCTATCTGAGCCTGCCAGCCTATAACCCATCTGG
GCCCTGATAGCTGGTGGCCAGCCCTGACCCCACCCCACCCTCCCTGGAACCTCTGATAGACA
CATCTGGCACACCAGCTCGCAAAGTCACCGTGAGGGTCTTGTGTTTGCTGAGTCAAAATTCC
TTGAAATCCAAGTCCTTAGAGACTCCTGCTCCCAAATTTACAGTCATAGACTTCTTCATGGC
TGTCTCCTTTATCCACAGAATGATTCCTTTGCTTCATTGCCCCATCCATCTGATCCTCCTCA
TCAGTGCAGCACAGGGCCCATGAGCAGTAGCTGCAGAGTCTCACATAGGTCTGGCACTGCCT
CTGACATGTCCGACCTTAGGCAAATGCTTGACTCTTCTGAGCTCAGTCTTGTCATGGCAAAA
TAAAGATAATAATAGTGTTTTTTTATGGAGTTAGCGTGAGGATGGAAAACAATAGCAAAATT
GATTAGACTATAAAAGGTCTCAACAAATAGTAGTAGATTTTATCATCCATTAATCCTTCCCT
CTCCTCTCTTACTCATCCCATCACGTATGCCTCTTAATTTTCCCTTACCTATAATAAGAGTT
ATTCCTCTTATTATATTCTTCTTATAGTGATTCTGGATATTAAAGTGGGAATGAGGGGCAGG
CCACTAACGAAGAAGATGTTTCTCAAAGAAGCGGGGGATCCACTAGTTCTAGAGCGGCCGTA
CTCGAGGTTTAAACAGTACCTTTAAGACCAATGACTTACAAGGCAGCTGTAGATCTTAGCCA
CTTTTTAAAAGAAAAGGGGGGACTGGAAGGGCTAATTCACTCCCAACGAAGACAAGATCTGC
TTTTTGCTTGTACTGGGTCTCTCTGGTTAGACCAGATCTGAGCCTGGGAGCTCTCTGGCTAA
CTAGGGAACCCACTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGTCAGTGTGGAAAA
TCTCTAGCAG
Underlined text: RSV
Double underlined text: 5' LTR
Underlined, italicized text: RRE
Bold, underlined text: ZNF410 shmiR in miR144
Bold text: Tomato
Italicized text: Beta-globin promoter and LCR
LV-LCR-ZBTB7A
(SEQ ID NO: 26; see FIG. 27 for schematic)
TTAATGTAGTCTTATGCAATACTCTTGTAGTCTTGCAACATGGTAACGATGAGTTAGCAACA
TGCCTTACAAGGAGAGAAAAAGCACCGTGCATGCCGATTGGTGGAAGTAAGGTGGTACGATC
GTGCCTTATTAGGAAGGCAACAGACGGGTCTGACATGGATTGGACGAACCACTGAATTGCCG
CATTGCAGAGATATTGTATTTAAGTGCCTAGCTCGATACATAAACGGGTCTCTCTGGTTAGA
CCAGATCTGAGCCTGGGAGCTCTCTGGCTAACTAGGGAACCCACTGCTTAAGCCTCAATAAA
GCTTGCCTTGAGTGCTTCAAGTAGTGTGTGCCCGTCTGTTGTGTGACTCTGGTAACTAGAGA
TCCCTCAGACCCTTTTAGTCAGTGTGGAAAATCTCTAGCAGTGGCGCCCGAACAGGGACTTG
AAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGA
AGGAGAGAGATGGGTGCGAGAGCGTCAGTATTAAGCGGGGGAGAATTAGATCGCGATGGGAA
AAAATTCGGTTAAGGCCAGGGGGAAAGAAAAAATATAAATTAAAACATATAGTATGGGCAAG
CAGGGAGCTAGAACGATTCGCAGTTAATCCTGGCCTGTTAGAAACATCAGAAGGCTGTAGAC
AAATACTGGGACAGCTACAACCATCCCTTCAGACAGGATCAGAAGAACTTAGATCATTATAT
AATACAGTAGCAACCCTCTATTGTGTGCATCAAAGGATAGAGATAAAAGACACCAAGGAAGC
TTTAGACAAGATAGAGGAAGAGCAAAACAAAAGTAAGACCACCGCACAGCAAGCGGCCGCTG
ATCTTCAGACCTGGAGGAGGAGATATGAGGGACAATTGGAGAAGTGAATTATATAAATATAA
AGTAGTAAAAATTGAACCATTAGGAGTAGCACCCACCAAGGCAAAGAGAAGAGTGGTGCAGA
GAGAAAAAAGAGCAGTGGGAATAGGAGCTTTGTTCCTTGGGTTCTTGGGAGCAGCAGGAAGC
ACTATGGGCGCAGCGTCAATGACGCTGACGGTACAGGCCAGACAATTATTGTCTGGTATAGT
GCAGCAGCAGAACAATTTGCTGAGGGCTATTGAGGCGCAACAGCATCTGTTGCAACTCACAG
TCTGGGGCATCAAGCAGCTCCAGGCAAGAATCCTGGCTGTGGAAAGATACCTAAAGGATCAA
CAGCTCCTGGGGATTTGGGGTTGCTCTGGAAAACTCATTTGCACCACTGCTGTGCCTTGGAA
TGCTAGTTGGAGTAATAAATCTCTGGAACAGATTTGGAATCACACGACCTGGATGGAGTGGG
ACAGAGAAATTAACAATTACACAAGCTTAATACACTCCTTAATTGAAGAATCGCAAAACCAG
CAAGAAAAGAATGAACAAGAATTATTGGAATTAGATAAATGGGCAAGTTTGTGGAATTGGTT
TAACATAACAAATTGGCTGTGGTATATAAAATTATTCATAATGATAGTAGGAGGCTTGGTAG
GTTTAAGAATAGTTTTTGCTGTACTTTCTATAGTGAATAGAGTTAGGCAGGGATATTCACCA
TTATCGTTTCAGACCCACCTCCCAACCCCGAGGGGACCCGACAGGCCCGAAGGAATAGAAGA
AGAAGGTGGAGAGAGAGACAGAGACAGATCCATTCGATTAGTGAACGGATCTCGACGGTATC
GGTTAACTTTTAAAAGAAAAGGGGGGATTGGGGGGTACAGTGCAGGGGAAAGAATAGTAGAC
ATAATAGCAACAGACATACAAACTAAAGAATTACAAAAACAAATTACAAAATTCAAAATTTT
ATCGATAAGCTTGATGGCCGCGCTTAATGCGCCGCTACAGGGCGCGTGGGGATACCCCCTAG
AGCCCCAGCTGGTTCTTTCCGCCTCAGAAGCCATAGAGCCCACCGCATCCCCAGCATGCCTG
CTATTGTCTTCCCAATCCTCCCCCTTGCTGTCCTGCCCCACCCCACCCCCCAGAATAGAATG
ACACCTACTCAGACAATGCGATGCAATTTCCTCATTTTATTAGGAAAGGACAGTGGGAGTGG
CACCTTCCAGGGTCAAGGAAGGCACGGGGGAGGGGCAAACAACAGATGGCTGGCAACTAGAA
GGCACAGTCGAGGCTGATCAGCGAGCTCTAGCATTTAGGTGACACTATAGAATAGGGCGCTC
TAGAACTAGTGGATCCCCCGGGGGAATTCGCCCTTACGCGTATCTCTCCTGTCCTCCTTGTC
AGGCTCCAGAGCTGGGGGTGCCCGGACTACGGGTACTTTTCATTCGCGCGTGTCTCATCGCA
AACTGCGCGAATGAAAAGTACCCGTCAGCCAGGGCCCCACTGGGGGCACAGGAAGCATGGCT
TGAAAAGCGACCTCGAGGGGGGGCCGCTTACTTGTACAGCTCGTCCATGCCGTACAGGAACA
GGTGGTGGCGGCCCTCGGAGCGCTCGTACTGTTCCACGATGGTGTAGTCCTCGTTGTGGGAG
GTGATGTCCAGCTTGGTGTCCACGTAGTAGTAGCCGGGCAGTTGCACGGGCTTCTTGGCCAT
GTAGATGGTCTTGAACTCCACCAGGTAGTGGCCGCCGTCCTTCAGCTTCAGGGCCTGGTGGA
TCTCGCCCTTCAGCACGCCGTCGCGGGGGTACAGGCGCTCGGTGGAGGCCTCCCAGCCCATG
GTCTTCTTCTGCATTACGGGGCCGTCGGGGGGGAAGTTGGTGCCGCGCATCTTCACCTTGTA
GATCAGCGTGCCGTCCTGCAGGGAGGAGTCCTGGGTCACGGTCACCAGACCGCCGTCCTCGA
AGTTCATCACGCGCTCCCACTTGAAGCCCTCGGGGAAGGACAGCTTCTTGTAATCGGGGATG
TCGGCGGGGTGCTTCACGTACGCCTTGGAGCCGTACATGAACTGGGGGGACAGGATGTCCCA
GGCGAAGGGCAGGGGGCCGCCCTTGGTCACCTTCAGCTTGGCGGTCTGGGTGCCCTCGTAGG
GGCGGCCCTCGCCCTCGCCCTCGATCTCGAACTCGTGGCCGTTCATGGAGCCCTCCATGCGC
ACCTTGAAGCGCATGAACTCTTTGATGACCTCCTCGCCCTTGCTCACCATGCCATCCACGTG
ATCGATGCTAGCTATGGTGTCTGTTTGAGGTTGCTAGTGAACACAGTTGTGTCAGAAGCAAA
TGTAAGCAATAGATGGCTCTGCCCTGACTTTTATGCCCAGCCCTGGCTCCTGCCCTCCCTGC
TCCTGGGAGTAGATTGGCCAACCCTAGGGTGTGGCTCCACAGGGTGAGGTCTAAGTGATGAC
AGCCGTACCTGTCCTTGGCTCTTCTGGCACTGGCTTAGGAGTTGGACTTCAAACCCTCAGCC
CTCCCTCTAAGATATATCTCTTGGCCCCATACCATCAGTACAAATTGCTACTAAAAACATCC
TCCTTTGCAAGTGTATTTACACGGTATCGATAAGCTTGAATTCCTGCAGCCCCCTTTTGCCA
CCTAGCTGTCCAGGGGTGCCTTAAAATGGCAAACAAGGTTTGTTTTCTTTTCCTGTTTTCAT
GCCTTCCTCTTCCATATCCTTGTTTCATATTAATACATGTGTATAGATCCTAAAAATCTATA
CACATGTATTAATAAAGCCTGATTCTGCCGCTTCTAGGTATAGAGGCCACCTGCAAGATAAA
TATTTGATTCACAATAACTAATCATTCTATGGCAATTGATAACAACAAATATATATATATAT
ATATATACGTATATGTGTATATATATATATATATATTCAGGAAATAATATATTCTAGAATAT
GTCACATTCTGTCTCAGGCATCCATTTTCTTTATGATGCCGTTTGAGGTGGAGTTTTAGTCA
GGTGGTCAGCTTCTCCTTTTTTTTGCCATCTGCCCTGTAAGCATCCTGCTGGGGACCCAGAT
AGGAGTCATCACTCTAGGCTGAGAACATCTGGGCACACACCCTAAGCCTCAGCATGACTCAT
CATGACTCAGCATTGCTGTGCTTGAGCCAGAAGGTTTGCTTAGAAGGTTACACAGAACCAGA
AGGCGGGGGTGGGGCACTGACCCCGACAGGGGCCTGGCCAGAACTGCTCATGCTTGGACTAT
GGGAGGTCACTAATGGAGACACACAGAAATGTAACAGGAACTAAGGAAAAACTGAAGCTTAT
TTAATCAGAGATGAGGATGCTGGAAGGGATAGAGGGAGCTGAGCTTGTAAAAAGTATAGTAA
TCATTCAGCAAATGGTTTTGAAGCACCTGCTGGATGCTAAACACTATTTTCAGTGCTTGAAT
CATAAATAAGAATAAAACATGTATCTTATTCCCCACAAGAGTCCAAGTAAAAAATAACAGTT
AATTATAATGTGCTCTGTCCCCCAGGCTGGAGTGCAGTGGCACGATCTCAGCTCACTGCAAC
CTCCGCCTCCCGGGGGGTTCAAGCAATTCTCCTGCCTCAGCCACCCTAATAGCTGGGATTAC
AGGTGCACACCACCATGCCAGGCTAATTTTTGTACTTTTTGTAGAGGTTTTGTACTTTTTGT
AGAGGCAGGGTATCACCATGTTGTCCAAGATGGTCTTGAACTCCTGAGCTCCAAGCAGTCCA
CCCACCTCAGCCTCCCAAAGTGCTGGGATTACAGGTGTGAGACACCATGCCCAGATTTTCCA
TATTTAATAGAGGTATTTATGGGATGGGGGAAAAGAATGTTTCTCTCACTGTGGATTATTTT
AGAGAGTGGAGAATGGTCAAGATTTTTTTAAAAATTAAGAAAACATAAGTTGGACCTTGAGA
AATGAAAATTTATTTTTTTGTTGGAGGATACCCATTCTCTATCTCCCATCAGGGCAAGCTGT
AAGGAACTGGCTAAGACACAGTGAGACAGAGTGACTTAGTCTTAGAGGCCCCACTGGTACGA
CGGTCACCAAGCTTTCATTAAAAAAAGTCTAACCAGCTGCATTCGACTTTGACTGCAGCAGC
TGGTTAGAAGGTTCTACTGGAGGAGGGTCCCAGCCCATTGCTAAATTAACATCAGGCTCTGA
GACTGGCAGTATATCTCTAACAGTGGTTGATGCTATCTTCTGGAACTTGCCTGCTACATTGA
GACCACTGACCCATACATAGGAAGCCCATAGCTCTGTCCTGAACTGTTAGGCCACTGGTCCA
GAGAGTGTGCATCTCCTTTGATCCTCATAATAACCCTATGAGATAGACACAATTATTACTCT
TACTTTATAGATGATGATCCTGAAAACATAGGAGTCAAGGCACTTGCCCCTAGCTGGGGGTA
TAGGGGAGCAGTCCCATGTAGTAGTAGAATGAAAAATGCTGCTATGCTGTGCCTCCCCCACC
TTTCCCATGTCTGCCCTCTACTCATGGTCTATCTCTCCTGGCTCCTGGGAGTCATGGACTCC
ACCCAGCACCACCAACCTGACCTAACCACCTATCTGAGCCTGCCAGCCTATAACCCATCTGG
GCCCTGATAGCTGGTGGCCAGCCCTGACCCCACCCCACCCTCCCTGGAACCTCTGATAGACA
CATCTGGCACACCAGCTCGCAAAGTCACCGTGAGGGTCTTGTGTTTGCTGAGTCAAAATTCC
TTGAAATCCAAGTCCTTAGAGACTCCTGCTCCCAAATTTACAGTCATAGACTTCTTCATGGC
TGTCTCCTTTATCCACAGAATGATTCCTTTGCTTCATTGCCCCATCCATCTGATCCTCCTCA
TCAGTGCAGCACAGGGCCCATGAGCAGTAGCTGCAGAGTCTCACATAGGTCTGGCACTGCCT
CTGACATGTCCGACCTTAGGCAAATGCTTGACTCTTCTGAGCTCAGTCTTGTCATGGCAAAA
TAAAGATAATAATAGTGTTTTTTTATGGAGTTAGCGTGAGGATGGAAAACAATAGCAAAATT
GATTAGACTATAAAAGGTCTCAACAAATAGTAGTAGATTTTATCATCCATTAATCCTTCCCT
CTCCTCTCTTACTCATCCCATCACGTATGCCTCTTAATTTTCCCTTACCTATAATAAGAGTT
ATTCCTCTTATTATATTCTTCTTATAGTGATTCTGGATATTAAAGTGGGAATGAGGGGCAGG
CCACTAACGAAGAAGATGTTTCTCAAAGAAGCGGGGGATCCACTAGTTCTAGAGCGGCCGTA
CTCGAGGTTTAAACAGTACCTTTAAGACCAATGACTTACAAGGCAGCTGTAGATCTTAGCCA
CTTTTTAAAAGAAAAGGGGGGACTGGAAGGGCTAATTCACTCCCAACGAAGACAAGATCTGC
TTTTTGCTTGTACTGGGTCTCTCTGGTTAGACCAGATCTGAGCCTGGGAGCTCTCTGGCTAA
CTAGGGAACCCACTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGTCAGTGTGGAAAA
TCTCTAGCAG
Underlined text: RSV
Double underlined text: 5' LTR
Underlined, italicized text: RRE
Bold, underlined text: ZBTB7A shmiR in miR144
Bold text: Tomato
Italicized text: Beta-globin promoter and LCR
LV-LCR-BCL11A-ZNF410
(SEQ ID NO: 27; see FIG. 27 for schematic)
TTAATGTAGTCTTATGCAATACTCTTGTAGTCTTGCAACATGGTAACGATGAGTTAGCAACA
TGCCTTACAAGGAGAGAAAAAGCACCGTGCATGCCGATTGGTGGAAGTAAGGTGGTACGATC
GTGCCTTATTAGGAAGGCAACAGACGGGTCTGACATGGATTGGACGAACCACTGAATTGCCG
CATTGCAGAGATATTGTATTTAAGTGCCTAGCTCGATACATAAACGGGTCTCTCTGGTTAGA
CCAGATCTGAGCCTGGGAGCTCTCTGGCTAACTAGGGAACCCACTGCTTAAGCCTCAATAAA
GCTTGCCTTGAGTGCTTCAAGTAGTGTGTGCCCGTCTGTTGTGTGACTCTGGTAACTAGAGA
TCCCTCAGACCCTTTTAGTCAGTGTGGAAAATCTCTAGCAGTGGCGCCCGAACAGGGACTTG
AAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGA
AGGAGAGAGATGGGTGCGAGAGCGTCAGTATTAAGCGGGGGAGAATTAGATCGCGATGGGAA
AAAATTCGGTTAAGGCCAGGGGGAAAGAAAAAATATAAATTAAAACATATAGTATGGGCAAG
CAGGGAGCTAGAACGATTCGCAGTTAATCCTGGCCTGTTAGAAACATCAGAAGGCTGTAGAC
AAATACTGGGACAGCTACAACCATCCCTTCAGACAGGATCAGAAGAACTTAGATCATTATAT
AATACAGTAGCAACCCTCTATTGTGTGCATCAAAGGATAGAGATAAAAGACACCAAGGAAGC
TTTAGACAAGATAGAGGAAGAGCAAAACAAAAGTAAGACCACCGCACAGCAAGCGGCCGCTG
ATCTTCAGACCTGGAGGAGGAGATATGAGGGACAATTGGAGAAGTGAATTATATAAATATAA
AGTAGTAAAAATTGAACCATTAGGAGTAGCACCCACCAAGGCAAAGAGAAGAGTGGTGCAGA
GAGAAAAAAGAGCAGTGGGAATAGGAGCTTTGTTCCTTGGGTTCTTGGGAGCAGCAGGAAGC
ACTATGGGCGCAGCGTCAATGACGCTGACGGTACAGGCCAGACAATTATTGTCTGGTATAGT
GCAGCAGCAGAACAATTTGCTGAGGGCTATTGAGGCGCAACAGCATCTGTTGCAACTCACAG
TCTGGGGCATCAAGCAGCTCCAGGCAAGAATCCTGGCTGTGGAAAGATACCTAAAGGATCAA
CAGCTCCTGGGGATTTGGGGTTGCTCTGGAAAACTCATTTGCACCACTGCTGTGCCTTGGAA
TGCTAGTTGGAGTAATAAATCTCTGGAACAGATTTGGAATCACACGACCTGGATGGAGTGGG
ACAGAGAAATTAACAATTACACAAGCTTAATACACTCCTTAATTGAAGAATCGCAAAACCAG
CAAGAAAAGAATGAACAAGAATTATTGGAATTAGATAAATGGGCAAGTTTGTGGAATTGGTT
TAACATAACAAATTGGCTGTGGTATATAAAATTATTCATAATGATAGTAGGAGGCTTGGTAG
GTTTAAGAATAGTTTTTGCTGTACTTTCTATAGTGAATAGAGTTAGGCAGGGATATTCACCA
TTATCGTTTCAGACCCACCTCCCAACCCCGAGGGGACCCGACAGGCCCGAAGGAATAGAAGA
AGAAGGTGGAGAGAGAGACAGAGACAGATCCATTCGATTAGTGAACGGATCTCGACGGTATC
GGTTAACTTTTAAAAGAAAAGGGGGGATTGGGGGGTACAGTGCAGGGGAAAGAATAGTAGAC
ATAATAGCAACAGACATACAAACTAAAGAATTACAAAAACAAATTACAAAATTCAAAATTTT
ATCGATAAGCTTGATGGCCGCGCTTAATGCGCCGCTACAGGGCGCGTGGGGATACCCCCTAG
AGCCCCAGCTGGTTCTTTCCGCCTCAGAAGCCATAGAGCCCACCGCATCCCCAGCATGCCTG
CTATTGTCTTCCCAATCCTCCCCCTTGCTGTCCTGCCCCACCCCACCCCCCAGAATAGAATG
ACACCTACTCAGACAATGCGATGCAATTTCCTCATTTTATTAGGAAAGGACAGTGGGAGTGG
CACCTTCCAGGGTCAAGGAAGGCACGGGGGAGGGGCAAACAACAGATGGCTGGCAACTAGAA
GGCACAGTCGAGGCTGATCAGCGAGCTCTAGCATTTAGGTGACACTATAGAATAGGGCGCTC
TAGAACTAGTGGATCCCCCGGGGGAATTCGCCCTTACGCGTATCTCTCCTGTCCTCCTTGTC
AGGCTCCAGAGCTGGGGGTGCCCGGACTGCTGAGCACTTAGTGTTTGTAGTGTCTCATCGCA
AACTTACAAACACTAAGTGCTCAGCCAGCCAGGGCCCCACTGGGGGCACAGGAAGCATGGCT
TGAAAAGCGACCTCGAGGACGCGTCGACGTCGGTGAGCATCCTTGTATCCAGCTGGCAGTCC
ATTCGTCATATCCCATCTGCCCTGGCCTAGAGCTGGTAAGCATGTGCCGCACTGCGCGATCG
AGTGTTGAATAACTCTACCACATGGAGTTATTCAACACTCGATCGCGCAGCGTGGCACTGCA
GGAGGCCAGGCCAAGAGCTTCTGTGGGGAAGTGAGATCCCCCGGGGGAATTCGATATCAAGC
TTATCGATACCGTCGACCTCGAGGGGGGGCCGCTTACTTGTACAGCTCGTCCATGCCGAGAG
TGATCCCGGCGGCGGTCACGAACTCCAGCAGGACCATGTGATCGCGCTTCTCGTTGGGGTCT
TTGCTCAGGGCGGACTGGTAGCTCAGGTAGTGGTTGTCGGGCAGCAGCACGGGGCCGTCGCC
GATGGGGGTGTTCTGCTGGTAGTGGTCGGCGAGCTGCACGCCGCCGTCCTCGATGTTGTGGC
GGATCTTGAAGTTGGCCTTGATGCCGTTCTTCTGCTTGTCGGCGGTGATATAGACGTTGTGG
CTGTTGTAGTTGTACTCCAGCTTGTGCCCCAGGATGTTGCCGTCCTCCTTGAAGTCGATGCC
CTTCAGCTCGATGCGGTTCACCAGGGTGTCGCCCTCGAACTTCACCTCGGCGCGGGTCTTGT
AGTTGCCGTCGTCCTTGAAGAAGATGGTGCGCTCCTGGACGTAGCCTTCGGGCATGGCGGAC
TTGAAGAAGTCGTGCTGCTTCATGTGGTCGGGGTAGCGGGCGAAGCACTGCAGGCCGTAGCC
CAGGGTGGTCACGAGGGTGGGCCAGGGCACGGGCAGCTTGCCGGTGGTGCAGATCAGCTTCA
GGGTCAGCTTGCCGTAGGTGGCATCGCCCTCGCCCTCGCCGGACACGCTGAACTTGTGGCCG
TTTACGTCGCCGTCCAGCTCGACCAGGATGGGCACCACCCCGGTGAACAGCTCCTCGCCCTT
GCTCACCATGCCATCCACGTGATCGATGCTAGCTATGGTGTCTGTTTGAGGTTGCTAGTGAA
CACAGTTGTGTCAGAAGCAAATGTAAGCAATAGATGGCTCTGCCCTGACTTTTATGCCCAGC
CCTGGCTCCTGCCCTCCCTGCTCCTGGGAGTAGATTGGCCAACCCTAGGGTGTGGCTCCACA
GGGTGAGGTCTAAGTGATGACAGCCGTACCTGTCCTTGGCTCTTCTGGCACTGGCTTAGGAG
TTGGACTTCAAACCCTCAGCCCTCCCTCTAAGATATATCTCTTGGCCCCATACCATCAGTAC
AAATTGCTACTAAAAACATCCTCCTTTGCAAGTGTATTTACACGGTATCGATAAGCTTGAAT
TCCTGCAGCCCCCTTTTGCCACCTAGCTGTCCAGGGGTGCCTTAAAATGGCAAACAAGGTTT
GTTTTCTTTTCCTGTTTTCATGCCTTCCTCTTCCATATCCTTGTTTCATATTAATACATGTG
TATAGATCCTAAAAATCTATACACATGTATTAATAAAGCCTGATTCTGCCGCTTCTAGGTAT
AGAGGCCACCTGCAAGATAAATATTTGATTCACAATAACTAATCATTCTATGGCAATTGATA
ACAACAAATATATATATATATATATATACGTATATGTGTATATATATATATATATATTCAGG
AAATAATATATTCTAGAATATGTCACATTCTGTCTCAGGCATCCATTTTCTTTATGATGCCG
TTTGAGGTGGAGTTTTAGTCAGGTGGTCAGCTTCTCCTTTTTTTTGCCATCTGCCCTGTAAG
CATCCTGCTGGGGACCCAGATAGGAGTCATCACTCTAGGCTGAGAACATCTGGGCACACACC
CTAAGCCTCAGCATGACTCATCATGACTCAGCATTGCTGTGCTTGAGCCAGAAGGTTTGCTT
AGAAGGTTACACAGAACCAGAAGGCGGGGGTGGGGCACTGACCCCGACAGGGGCCTGGCCAG
AACTGCTCATGCTTGGACTATGGGAGGTCACTAATGGAGACACACAGAAATGTAACAGGAAC
TAAGGAAAAACTGAAGCTTATTTAATCAGAGATGAGGATGCTGGAAGGGATAGAGGGAGCTG
AGCTTGTAAAAAGTATAGTAATCATTCAGCAAATGGTTTTGAAGCACCTGCTGGATGCTAAA
CACTATTTTCAGTGCTTGAATCATAAATAAGAATAAAACATGTATCTTATTCCCCACAAGAG
TCCAAGTAAAAAATAACAGTTAATTATAATGTGCTCTGTCCCCCAGGCTGGAGTGCAGTGGC
ACGATCTCAGCTCACTGCAACCTCCGCCTCCCGGGGGGTTCAAGCAATTCTCCTGCCTCAGC
CACCCTAATAGCTGGGATTACAGGTGCACACCACCATGCCAGGCTAATTTTTGTACTTTTTG
TAGAGGTTTTGTACTTTTTGTAGAGGCAGGGTATCACCATGTTGTCCAAGATGGTCTTGAAC
TCCTGAGCTCCAAGCAGTCCACCCACCTCAGCCTCCCAAAGTGCTGGGATTACAGGTGTGAG
ACACCATGCCCAGATTTTCCATATTTAATAGAGGTATTTATGGGATGGGGGAAAAGAATGTT
TCTCTCACTGTGGATTATTTTAGAGAGTGGAGAATGGTCAAGATTTTTTTAAAAATTAAGAA
AACATAAGTTGGACCTTGAGAAATGAAAATTTATTTTTTTGTTGGAGGATACCCATTCTCTA
TCTCCCATCAGGGCAAGCTGTAAGGAACTGGCTAAGACACAGTGAGACAGAGTGACTTAGTC
TTAGAGGCCCCACTGGTACGACGGTCACCAAGCTTTCATTAAAAAAAGTCTAACCAGCTGCA
TTCGACTTTGACTGCAGCAGCTGGTTAGAAGGTTCTACTGGAGGAGGGTCCCAGCCCATTGC
TAAATTAACATCAGGCTCTGAGACTGGCAGTATATCTCTAACAGTGGTTGATGCTATCTTCT
GGAACTTGCCTGCTACATTGAGACCACTGACCCATACATAGGAAGCCCATAGCTCTGTCCTG
AACTGTTAGGCCACTGGTCCAGAGAGTGTGCATCTCCTTTGATCCTCATAATAACCCTATGA
GATAGACACAATTATTACTCTTACTTTATAGATGATGATCCTGAAAACATAGGAGTCAAGGC
ACTTGCCCCTAGCTGGGGGTATAGGGGAGCAGTCCCATGTAGTAGTAGAATGAAAAATGCTG
CTATGCTGTGCCTCCCCCACCTTTCCCATGTCTGCCCTCTACTCATGGTCTATCTCTCCTGG
CTCCTGGGAGTCATGGACTCCACCCAGCACCACCAACCTGACCTAACCACCTATCTGAGCCT
GCCAGCCTATAACCCATCTGGGCCCTGATAGCTGGTGGCCAGCCCTGACCCCACCCCACCCT
CCCTGGAACCTCTGATAGACACATCTGGCACACCAGCTCGCAAAGTCACCGTGAGGGTCTTG
TGTTTGCTGAGTCAAAATTCCTTGAAATCCAAGTCCTTAGAGACTCCTGCTCCCAAATTTAC
AGTCATAGACTTCTTCATGGCTGTCTCCTTTATCCACAGAATGATTCCTTTGCTTCATTGCC
CCATCCATCTGATCCTCCTCATCAGTGCAGCACAGGGCCCATGAGCAGTAGCTGCAGAGTCT
CACATAGGTCTGGCACTGCCTCTGACATGTCCGACCTTAGGCAAATGCTTGACTCTTCTGAG
CTCAGTCTTGTCATGGCAAAATAAAGATAATAATAGTGTTTTTTTATGGAGTTAGCGTGAGG
ATGGAAAACAATAGCAAAATTGATTAGACTATAAAAGGTCTCAACAAATAGTAGTAGATTTT
ATCATCCATTAATCCTTCCCTCTCCTCTCTTACTCATCCCATCACGTATGCCTCTTAATTTT
CCCTTACCTATAATAAGAGTTATTCCTCTTATTATATTCTTCTTATAGTGATTCTGGATATT
AAAGTGGGAATGAGGGGCAGGCCACTAACGAAGAAGATGTTTCTCAAAGAAGCGGGGGATCC
ACTAGTTCTAGAGCGGCCGTACTCGAGGTTTAAACAGTACCTTTAAGACCAATGACTTACAA
GGCAGCTGTAGATCTTAGCCACTTTTTAAAAGAAAAGGGGGGACTGGAAGGGCTAATTCACT
CCCAACGAAGACAAGATCTGCTTTTTGCTTGTACTGGGTCTCTCTGGTTAGACCAGATCTGA
GCCTGGGAGCTCTCTGGCTAACTAGGGAACCCACTGCTTAAGCCTCAATAAAGCTTGCCTTG
AGTGCTTCAAGTAGTGTGTGCCCGTCTGTTGTGTGACTCTGGTAACTAGAGATCCCTCAGAC
CCTTTTAGTCAGTGTGGAAAATCTCTAGCAG
Underlined text: RSV
Double underlined text: 5' LTR
Underlined, italicized text: RRE
Bold, double underlined text: ZNF410 shmiR in miR 144
Bold, underlined text: BCL11A shmiR in miR223
Bold text: Venus
Italicized text: Beta-globin promoter and LCR
LV-LCR-BCL11A-ZBTB7A
(SEQ ID NO: 28; see FIG. 27 for schematic)
TTAATGTAGTCTTATGCAATACTCTTGTAGTCTTGCAACATGGTAACGATGAGTTAGCAACA
TGCCTTACAAGGAGAGAAAAAGCACCGTGCATGCCGATTGGTGGAAGTAAGGTGGTACGATC
GTGCCTTATTAGGAAGGCAACAGACGGGTCTGACATGGATTGGACGAACCACTGAATTGCCG
CATTGCAGAGATATTGTATTTAAGTGCCTAGCTCGATACATAAACGGGTCTCTCTGGTTAGA
CCAGATCTGAGCCTGGGAGCTCTCTGGCTAACTAGGGAACCCACTGCTTAAGCCTCAATAAA
GCTTGCCTTGAGTGCTTCAAGTAGTGTGTGCCCGTCTGTTGTGTGACTCTGGTAACTAGAGA
TCCCTCAGACCCTTTTAGTCAGTGTGGAAAATCTCTAGCAGTGGCGCCCGAACAGGGACTTG
AAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGA
AGGAGAGAGATGGGTGCGAGAGCGTCAGTATTAAGCGGGGGAGAATTAGATCGCGATGGGAA
AAAATTCGGTTAAGGCCAGGGGGAAAGAAAAAATATAAATTAAAACATATAGTATGGGCAAG
CAGGGAGCTAGAACGATTCGCAGTTAATCCTGGCCTGTTAGAAACATCAGAAGGCTGTAGAC
AAATACTGGGACAGCTACAACCATCCCTTCAGACAGGATCAGAAGAACTTAGATCATTATAT
AATACAGTAGCAACCCTCTATTGTGTGCATCAAAGGATAGAGATAAAAGACACCAAGGAAGC
TTTAGACAAGATAGAGGAAGAGCAAAACAAAAGTAAGACCACCGCACAGCAAGCGGCCGCTG
ATCTTCAGACCTGGAGGAGGAGATATGAGGGACAATTGGAGAAGTGAATTATATAAATATAA
AGTAGTAAAAATTGAACCATTAGGAGTAGCACCCACCAAGGCAAAGAGAAGAGTGGTGCAGA
GAGAAAAAAGAGCAGTGGGAATAGGAGCTTTGTTCCTTGGGTTCTTGGGAGCAGCAGGAAGC
ACTATGGGCGCAGCGTCAATGACGCTGACGGTACAGGCCAGACAATTATTGTCTGGTATAGT
GCAGCAGCAGAACAATTTGCTGAGGGCTATTGAGGCGCAACAGCATCTGTTGCAACTCACAG
TCTGGGGCATCAAGCAGCTCCAGGCAAGAATCCTGGCTGTGGAAAGATACCTAAAGGATCAA
CAGCTCCTGGGGATTTGGGGTTGCTCTGGAAAACTCATTTGCACCACTGCTGTGCCTTGGAA
TGCTAGTTGGAGTAATAAATCTCTGGAACAGATTTGGAATCACACGACCTGGATGGAGTGGG
ACAGAGAAATTAACAATTACACAAGCTTAATACACTCCTTAATTGAAGAATCGCAAAACCAG
CAAGAAAAGAATGAACAAGAATTATTGGAATTAGATAAATGGGCAAGTTTGTGGAATTGGTT
TAACATAACAAATTGGCTGTGGTATATAAAATTATTCATAATGATAGTAGGAGGCTTGGTAG
GTTTAAGAATAGTTTTTGCTGTACTTTCTATAGTGAATAGAGTTAGGCAGGGATATTCACCA
TTATCGTTTCAGACCCACCTCCCAACCCCGAGGGGACCCGACAGGCCCGAAGGAATAGAAGA
AGAAGGTGGAGAGAGAGACAGAGACAGATCCATTCGATTAGTGAACGGATCTCGACGGTATC
GGTTAACTTTTAAAAGAAAAGGGGGGATTGGGGGGTACAGTGCAGGGGAAAGAATAGTAGAC
ATAATAGCAACAGACATACAAACTAAAGAATTACAAAAACAAATTACAAAATTCAAAATTTT
ATCGATAAGCTTGATGGCCGCGCTTAATGCGCCGCTACAGGGCGCGTGGGGATACCCCCTAG
AGCCCCAGCTGGTTCTTTCCGCCTCAGAAGCCATAGAGCCCACCGCATCCCCAGCATGCCTG
CTATTGTCTTCCCAATCCTCCCCCTTGCTGTCCTGCCCCACCCCACCCCCCAGAATAGAATG
ACACCTACTCAGACAATGCGATGCAATTTCCTCATTTTATTAGGAAAGGACAGTGGGAGTGG
CACCTTCCAGGGTCAAGGAAGGCACGGGGGAGGGGCAAACAACAGATGGCTGGCAACTAGAA
GGCACAGTCGAGGCTGATCAGCGAGCTCTAGCATTTAGGTGACACTATAGAATAGGGCGCTC
TAGAACTAGTGGATCCCCCGGGGGAATTCGCCCTTACGCGTATCTCTCCTGTCCTCCTTGTC
AGGCTCCAGAGCTGGGGGTGCCCGGACTACGGGTACTTTTCATTCGCGCGTGTCTCATCGCA
AACTGCGCGAATGAAAAGTACCCGTCAGCCAGGGCCCCACTGGGGGCACAGGAAGCATGGCT
TGAAAAGCGACCTCGAGGACGCGTCGACGTCGGTGAGCATCCTTGTATCCAGCTGGCAGTCC
ATTCGTCATATCCCATCTGCCCTGGCCTAGAGCTGGTAAGCATGTGCCGCACTGCGCGATCG
AGTGTTGAATAACTCTACCACATGGAGTTATTCAACACTCGATCGCGCAGCGTGGCACTGCA
GGAGGCCAGGCCAAGAGCTTCTGTGGGGAAGTGAGATCCCCCGGGGGAATTCGATATCAAGC
TTATCGATACCGTCGACCTCGAGGGGGGGCCGCTTACTTGTACAGCTCGTCCATGCCGAGAG
TGATCCCGGCGGCGGTCACGAACTCCAGCAGGACCATGTGATCGCGCTTCTCGTTGGGGTCT
TTGCTCAGGGCGGACTGGTAGCTCAGGTAGTGGTTGTCGGGCAGCAGCACGGGGCCGTCGCC
GATGGGGGTGTTCTGCTGGTAGTGGTCGGCGAGCTGCACGCCGCCGTCCTCGATGTTGTGGC
GGATCTTGAAGTTGGCCTTGATGCCGTTCTTCTGCTTGTCGGCGGTGATATAGACGTTGTGG
CTGTTGTAGTTGTACTCCAGCTTGTGCCCCAGGATGTTGCCGTCCTCCTTGAAGTCGATGCC
CTTCAGCTCGATGCGGTTCACCAGGGTGTCGCCCTCGAACTTCACCTCGGCGCGGGTCTTGT
AGTTGCCGTCGTCCTTGAAGAAGATGGTGCGCTCCTGGACGTAGCCTTCGGGCATGGCGGAC
TTGAAGAAGTCGTGCTGCTTCATGTGGTCGGGGTAGCGGGCGAAGCACTGCAGGCCGTAGCC
CAGGGTGGTCACGAGGGTGGGCCAGGGCACGGGCAGCTTGCCGGTGGTGCAGATCAGCTTCA
GGGTCAGCTTGCCGTAGGTGGCATCGCCCTCGCCCTCGCCGGACACGCTGAACTTGTGGCCG
TTTACGTCGCCGTCCAGCTCGACCAGGATGGGCACCACCCCGGTGAACAGCTCCTCGCCCTT
GCTCACCATGCCATCCACGTGATCGATGCTAGCTATGGTGTCTGTTTGAGGTTGCTAGTGAA
CACAGTTGTGTCAGAAGCAAATGTAAGCAATAGATGGCTCTGCCCTGACTTTTATGCCCAGC
CCTGGCTCCTGCCCTCCCTGCTCCTGGGAGTAGATTGGCCAACCCTAGGGTGTGGCTCCACA
GGGTGAGGTCTAAGTGATGACAGCCGTACCTGTCCTTGGCTCTTCTGGCACTGGCTTAGGAG
TTGGACTTCAAACCCTCAGCCCTCCCTCTAAGATATATCTCTTGGCCCCATACCATCAGTAC
AAATTGCTACTAAAAACATCCTCCTTTGCAAGTGTATTTACACGGTATCGATAAGCTTGAAT
TCCTGCAGCCCCCTTTTGCCACCTAGCTGTCCAGGGGTGCCTTAAAATGGCAAACAAGGTTT
GTTTTCTTTTCCTGTTTTCATGCCTTCCTCTTCCATATCCTTGTTTCATATTAATACATGTG
TATAGATCCTAAAAATCTATACACATGTATTAATAAAGCCTGATTCTGCCGCTTCTAGGTAT
AGAGGCCACCTGCAAGATAAATATTTGATTCACAATAACTAATCATTCTATGGCAATTGATA
ACAACAAATATATATATATATATATATACGTATATGTGTATATATATATATATATATTCAGG
AAATAATATATTCTAGAATATGTCACATTCTGTCTCAGGCATCCATTTTCTTTATGATGCCG
TTTGAGGTGGAGTTTTAGTCAGGTGGTCAGCTTCTCCTTTTTTTTGCCATCTGCCCTGTAAG
CATCCTGCTGGGGACCCAGATAGGAGTCATCACTCTAGGCTGAGAACATCTGGGCACACACC
CTAAGCCTCAGCATGACTCATCATGACTCAGCATTGCTGTGCTTGAGCCAGAAGGTTTGCTT
AGAAGGTTACACAGAACCAGAAGGCGGGGGTGGGGCACTGACCCCGACAGGGGCCTGGCCAG
AACTGCTCATGCTTGGACTATGGGAGGTCACTAATGGAGACACACAGAAATGTAACAGGAAC
TAAGGAAAAACTGAAGCTTATTTAATCAGAGATGAGGATGCTGGAAGGGATAGAGGGAGCTG
AGCTTGTAAAAAGTATAGTAATCATTCAGCAAATGGTTTTGAAGCACCTGCTGGATGCTAAA
CACTATTTTCAGTGCTTGAATCATAAATAAGAATAAAACATGTATCTTATTCCCCACAAGAG
TCCAAGTAAAAAATAACAGTTAATTATAATGTGCTCTGTCCCCCAGGCTGGAGTGCAGTGGC
ACGATCTCAGCTCACTGCAACCTCCGCCTCCCGGGGGGTTCAAGCAATTCTCCTGCCTCAGC
CACCCTAATAGCTGGGATTACAGGTGCACACCACCATGCCAGGCTAATTTTTGTACTTTTTG
TAGAGGTTTTGTACTTTTTGTAGAGGCAGGGTATCACCATGTTGTCCAAGATGGTCTTGAAC
TCCTGAGCTCCAAGCAGTCCACCCACCTCAGCCTCCCAAAGTGCTGGGATTACAGGTGTGAG
ACACCATGCCCAGATTTTCCATATTTAATAGAGGTATTTATGGGATGGGGGAAAAGAATGTT
TCTCTCACTGTGGATTATTTTAGAGAGTGGAGAATGGTCAAGATTTTTTTAAAAATTAAGAA
AACATAAGTTGGACCTTGAGAAATGAAAATTTATTTTTTTGTTGGAGGATACCCATTCTCTA
TCTCCCATCAGGGCAAGCTGTAAGGAACTGGCTAAGACACAGTGAGACAGAGTGACTTAGTC
TTAGAGGCCCCACTGGTACGACGGTCACCAAGCTTTCATTAAAAAAAGTCTAACCAGCTGCA
TTCGACTTTGACTGCAGCAGCTGGTTAGAAGGTTCTACTGGAGGAGGGTCCCAGCCCATTGC
TAAATTAACATCAGGCTCTGAGACTGGCAGTATATCTCTAACAGTGGTTGATGCTATCTTCT
GGAACTTGCCTGCTACATTGAGACCACTGACCCATACATAGGAAGCCCATAGCTCTGTCCTG
AACTGTTAGGCCACTGGTCCAGAGAGTGTGCATCTCCTTTGATCCTCATAATAACCCTATGA
GATAGACACAATTATTACTCTTACTTTATAGATGATGATCCTGAAAACATAGGAGTCAAGGC
ACTTGCCCCTAGCTGGGGGTATAGGGGAGCAGTCCCATGTAGTAGTAGAATGAAAAATGCTG
CTATGCTGTGCCTCCCCCACCTTTCCCATGTCTGCCCTCTACTCATGGTCTATCTCTCCTGG
CTCCTGGGAGTCATGGACTCCACCCAGCACCACCAACCTGACCTAACCACCTATCTGAGCCT
GCCAGCCTATAACCCATCTGGGCCCTGATAGCTGGTGGCCAGCCCTGACCCCACCCCACCCT
CCCTGGAACCTCTGATAGACACATCTGGCACACCAGCTCGCAAAGTCACCGTGAGGGTCTTG
TGTTTGCTGAGTCAAAATTCCTTGAAATCCAAGTCCTTAGAGACTCCTGCTCCCAAATTTAC
AGTCATAGACTTCTTCATGGCTGTCTCCTTTATCCACAGAATGATTCCTTTGCTTCATTGCC
CCATCCATCTGATCCTCCTCATCAGTGCAGCACAGGGCCCATGAGCAGTAGCTGCAGAGTCT
CACATAGGTCTGGCACTGCCTCTGACATGTCCGACCTTAGGCAAATGCTTGACTCTTCTGAG
CTCAGTCTTGTCATGGCAAAATAAAGATAATAATAGTGTTTTTTTATGGAGTTAGCGTGAGG
ATGGAAAACAATAGCAAAATTGATTAGACTATAAAAGGTCTCAACAAATAGTAGTAGATTTT
ATCATCCATTAATCCTTCCCTCTCCTCTCTTACTCATCCCATCACGTATGCCTCTTAATTTT
CCCTTACCTATAATAAGAGTTATTCCTCTTATTATATTCTTCTTATAGTGATTCTGGATATT
AAAGTGGGAATGAGGGGCAGGCCACTAACGAAGAAGATGTTTCTCAAAGAAGCGGGGGATCC
ACTAGTTCTAGAGCGGCCGTACTCGAGGTTTAAACAGTACCTTTAAGACCAATGACTTACAA
GGCAGCTGTAGATCTTAGCCACTTTTTAAAAGAAAAGGGGGGACTGGAAGGGCTAATTCACT
CCCAACGAAGACAAGATCTGCTTTTTGCTTGTACTGGGTCTCTCTGGTTAGACCAGATCTGA
GCCTGGGAGCTCTCTGGCTAACTAGGGAACCCACTGCTTAAGCCTCAATAAAGCTTGCCTTG
AGTGCTTCAAGTAGTGTGTGCCCGTCTGTTGTGTGACTCTGGTAACTAGAGATCCCTCAGAC
CCTTTTAGTCAGTGTGGAAAATCTCTAGCAG
Underlined text: RSV
Double underlined text: 5' LTR
Underlined, italicized text: RRE
Bold, double underlined text: ZBTB7A shmiR in miR 144
Bold, underlined text: BCL11A shmiR in miR223
Bold text: Venus
Italicized text: Beta-globin promoter and LCR

1. A nucleic acid vector encoding a first and at least a second microRNA-adapted short hairpin RNA (shmIR).

2. The nucleic acid vector of claim 1, wherein the first shmIR comprises a first short hairpin mRNA (shRNA) embedded in a framework of a first miRNA sequence, and the at least second shmiR comprises a second shRNA embedded in a framework of a second miRNA sequence.

3. The nucleic acid vector of claim 2, wherein the first miRNA sequence and/or second miRNA comprises a sequence from miR223 or miR144.

4. The nucleic acid vector of claim 3, wherein the sequence from miR223 or miR144 comprises one or more of: a first segment, a loop segment and/or a second segment.

5. The nucleic acid vector of claim 4, wherein the first segment of the miR223 miRNA comprises the sequence of:
(SEQ ID NO: 1)
GATCTCACTTCCCCACAGAAGCTCTTGGCCTGGCCTCCTGCAGTGCCAC
GCT.

6. The nucleic acid vector of claim 4, wherein the miR223 loop segment comprises the sequence of: CTCCATGTGGTAGAG (SEQ ID NO: 2).

7. The nucleic acid vector of claim 4, wherein the second segment of the miR223 miRNA comprises the sequence of:
(SEQ ID NO: 3)
AGTGCGGCACATGCTTACCAGCTCTAGGCCAGGGCAGATGGGATATGAC
GAATGGACTGCCAGCTGGATACAAGGATGCTCACC.

8. The nucleic acid vector of claim 4, the sequence of miR223 comprises each of SEQ ID Nos: 1, 2, and 3.

9. The nucleic acid vector of claim 4, the first segment of the miR144 miRNA comprises the sequence of:
(SEQ ID NO: 7)
CGCTTTTCAAGCCATGCTTCCTGTGCCCCCAGTGGGGCCCTGGCTG.

10. The nucleic acid vector of claim 4, wherein the loop segment comprises a segment of miR144.

11. The nucleic acid vector of claim 4, wherein the miR144 loop segment comprises the sequence ofAGTTTGCGATGAGACAC (SEQ ID NO: 8).

12. The nucleic acid vector of claim 4, wherein the second segment of the miR144 miRNA comprises the sequence of:
(SEQ ID NO: 9)
AGTCCGGGCACCCCCAGCTCTGGAGCCTGACAAGGAGGACAGGAGAGAT.

13. The nucleic acid vector of any one of claims 1-12, wherein the first miRNA sequence comprises miR223 and the second miRNA sequence comprises miR144.

14. The nucleic acid vector of claim 2, wherein the first shmiR comprises a first segment from the first miRNA, a first BCL11a segment, a loop segment of the first miRNA sequence, a second BCL11a segment, and a second segment from the first miRNA arranged in tandem in a 5′ to 3′ direction,
wherein the loop segment is between and directly linked to the first and second BCL11a segments, and
wherein the sequence of the second BCL11A segment is complementary to the sequence of the first BCL11a segment, and
wherein the sequence of the first segment of the first miRNA is complementary to the sequence of the second segment of the first miRNA, and
wherein (i) the first and second segments of the first miRNA and (ii) the first and second BCL11a segments base pair to form a hairpin loop, with the loop segment of the first miRNA forming the loop portion of the hairpin loop thus formed.

15. The nucleic acid vector of claim 2, wherein the second shmiR comprises a first segment from the at least second miRNA, a first ZNF410 or ZBTB7A segment, a loop segment of the at least second miRNA, a second, complementary ZNF410 or ZBTB7A segment, and a second segment of the at least second miRNA arranged in tandem in a 5′ to 3′ direction,
wherein the loop segment is between and directly linked to the first and second ZNF410 or ZBTB7A segments, and
wherein the sequence of the first segment of the at least second miRNA is complementary to the sequence of the second segment of the at least second miRNA,
wherein the sequence of the first segment of ZNF410 or ZBTB7a is complementary to the second segment of ZNF410 or ZBTB7a and,
wherein (i) the first and third segments of the at least second miRNA and (ii) the first and second ZNF410 or ZBTB7 segments base pair to form a hairpin loop, with the loop segment of the at least second miRNA forming the loop portion of the hairpin loop thus formed.

16. The nucleic acid vector of any one of claims 1-15, wherein the first shRNA comprises a BCL11a targeting sequence.

17. The nucleic acid vector of any one of claims 1-16, wherein the second shRNA comprises a ZNF410 targeting sequence or a ZBTB7A targeting sequence.

18. The nucleic acid vector of claim 16 or 17, wherein the first and second BCL11a, ZNF410 or ZBTB7A segments are each 18-25 nucleotides long.

19. The nucleic acid vector of claim 16, wherein the first BCL11A sequence comprises the sequence of: GCGCGATCGAGTGTTGAATAA (SEQ ID NO: 4) and the second BCL11 A sequence comprises the sequence of: TTATTCAACACTCGATCGCGC (SEQ ID NO: 5), wherein the first and second BCL11A sequence are complementary.

20. The nucleic acid vector of claim 16, wherein the BCL11A sequence in an miR223 framework comprises the sequence of:
(SEQ ID NO: 6)
GATCTCACTTCCCCACAGAAGCTCTTGGCCTGGCCTCCTGCAGTGCCACGCTGCGCGATCGA 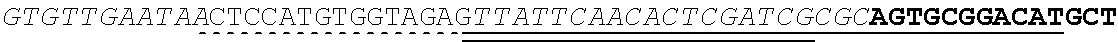
TACCAGCTCTAGGCCAGGGCAGATGGGATATGACGAATGGACTGCCAGCTGGATACAAGGAT
GCTCACC.
bold, underlined text: miR223 backbone
italicized text: BCL11A passenger strand sequence 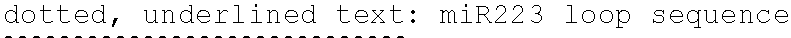
italics, double underlined text: BCL11A guide strand sequence

21. The nucleic acid vector of claim 17, wherein the first ZNF410 sequence comprises the sequence of: GCTGAGCACTTAGTGTTTGTA (SEQ ID NO: 10) and the second ZNF410 sequence comprises the sequence of: TACAAACACTAAGTGCTCAGC (SEQ ID NO: 11), wherein the first and second ZNF410 sequence are complementary.

22. The nucleic acid vector of claim 21, wherein the ZNF410 sequence in an miR144 framework comprises the sequence of:
(SEQ ID NO: 12)
CGCTTTTCAAGCCATGCTTCCTGTGCCCCCAGTGGGGCCCTGGCTGGCTGAGCACTTAGTGT 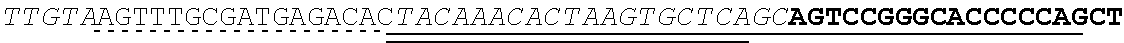
CTGGAGCCTGACAAGGAGGACAGGAGAGAT.
bold, underlined text: miR144 backbone
italicized text: ZNF410 passenger strand sequence 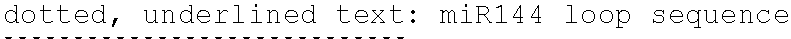
italics, double underlined text: ZNF410 guide strand sequence

23. The nucleic acid vector of claim 21, wherein the first ZBTB7A sequence comprises the sequence of: ACGGGTACTTTTCATTCGCGC (SEQ ID NO: 13) and the second ZBTB7A sequence comprises the sequence of: GCGCGAATGAAAAGTACCCGT (SEQ ID NO: 14), wherein the first and second ZBTB7A sequence are complementary.

24. The nucleic acid vector of claim 23, wherein the ZBTB7A sequence in an miR144 framework comprises the sequence of:
(SEQ ID NO: 15)
CGCTTTTCAAGCCATGCTTCCTGTGCCCCCAGTGGGGCCCTGGCTGACGGGTACTTTTCATT 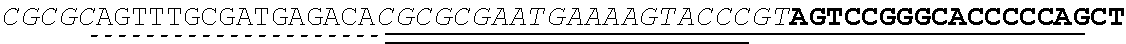
CTGGAGCCTGACAAGGAGGACAGGAGAGAT.
bold, underlined text: miR144 backbone
italicized text: ZBTB7A passenger strand sequence 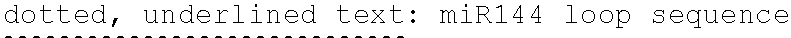
italics, double underlined text: ZBTB7A guide strand sequence

25. The nucleic acid vector of any one of claims 1-24, wherein the first shmiR and the second shmiR do not undergo homologous recombination when introduced into a cell.

26. An RNA transcript expressed from the nucleic acid vector of any one of claims 1-25.

27. An RNA transcript comprising a first and at least a second microRNA-adapted short hairpin RNA (shmiR).

28. The RNA transcript of claim 27, wherein the first shmiR comprises a first short hairpin mRNA (shRNA) embedded in a framework of a first miRNA sequence, and the at least second shmiR comprises a second shRNA embedded in a framework of a second miRNA sequence.

29. The RNA transcript of claim 27 or 28, wherein the first shmiR comprises a first segment from the first miRNA, a first BCL11a segment, a loop segment of the first miRNA sequence, a second BCL11a segment, and a second segment from the first miRNA arranged in tandem in a 5′ to 3′ direction,
wherein the loop segment is between and directly linked to the first and second BCL11a segments, and
wherein the sequence of the second BCL11A segment is complementary to the sequence of the first BCL11a segment, and
wherein the sequence of the first segment of the first miRNA is complementary to the sequence of the second segment of the first miRNA, and
wherein (i) the first and second segments of the first miRNA and (ii) the first and second BCL11a segments base pair to form a hairpin loop, with the loop segment of the first miRNA forming the loop portion of the hairpin loop thus formed.

30. The RNA transcript of any one of claims 27-29, wherein the second shmiR comprises a first segment from the at least second miRNA, a first ZNF410 or ZBTB7A segment, a loop segment of the at least second miRNA, a second, complementary ZNF410 or ZBTB7A segment, and a second segment of the at least second miRNA arranged in tandem in a 5′ to 3′ direction,
wherein the loop segment is between and directly linked to the first and second ZNF410 or ZBTB7A segments, and
wherein the sequence of the first segment of the at least second miRNA is complementary to the sequence of the second segment of the at least second miRNA,
wherein the sequence of the first segment of ZNF410 or ZBTB7a is complementary to the second segment of ZNF410 or ZBTB7a and,
wherein (i) the first and third segments of the at least second miRNA and (ii) the first and second ZNF410 or ZBTB7 segments base pair to form a hairpin loop, with the loop segment of the at least second miRNA forming the loop portion of the hairpin loop thus formed.

31. The RNA transcript of any one of claims 27-30, wherein the first miRNA sequence comprises an miR223 sequence.

32. The RNA transcript of claim 31, wherein the miR223 sequences comprises one or more of: SEQ ID NOs: 1, 2, and/or 3.

33. The RNA transcript of any one of claims 27-32, wherein the at least second miRNA sequence comprises an miR144 sequence.

34. The RNA transcript of claim 33, wherein the miR144 sequence comprises one or more of: SEQ ID Nos: 7, 8 and/or 9.

35. The RNA transcript of any one of claims 27-34, wherein the first miRNA sequence comprises miR223 and the second miRNA sequence comprises miR144.

36. The RNA transcript of any one of claims 27-35, wherein the first shRNA comprises a BCL11a targeting sequence.

37. The RNA transcript of any one of claims 27-36, wherein the second shRNA comprises a ZNF410 targeting sequence or a ZBTB7A targeting sequence.

38. The RNA transcript of claim 36 or 37, wherein the first and second BCL11a, ZNF410 or ZBTB7A segments are each 18-25 nucleotides long.

39. The RNA transcript of claim 38, wherein either the first or the second shmiR comprises a BCL11a sequence in an miR223 framework.

40. The RNA transcript of claim 38, wherein the BCL11a sequence in an miR223 framework comprises the sequence of: SEQ ID NO: 6.

41. The RNA transcript of any one of claims 27-40, wherein either the first or the second shmiR comprises a ZNF410 sequence in an miR144 framework.

42. The RNA transcript of claim 41, the ZNF410 sequence in an miR144 framework comprises the sequence of: SEQ ID NO: 12.

43. The RNA transcript of any one of claims 27-42, wherein either the first or the second shmiR comprises a ZBTB7A sequence in an miR144 framework.

44. The RNA transcript of claim 43, wherein the ZBTB7A sequence in an miR144 framework comprises the sequence of: SEQ ID NO:15.

45. The RNA transcript of any one of claims 36-44, wherein the first and second BCL11a, ZNF410 or ZBTB7A segments are complementary.

46. The RNA transcript of any one of claims 27-45, wherein the first shmiR and the second shmiR do not undergo homologous recombination when introduced into a cell.

47. A method for treating or reducing at least one symptom of a hemoglobinopathy, the method comprising: administering a vector of any one of claims 1-19 or an RNA transcript of any one of claims 20-37 to a subject in need thereof, thereby treating or reducing at least one symptom of the hemoglobinopathy.

48. The method of claim 38, wherein the hemoglobinopathy is sickle cell anemia, SCD or thalassemia.

49. The method of claim 38 or 39, wherein the at least one symptom of the hemoglobinopathy comprises the presence or number of sickle cells in a blood sample obtained from the subject.

50. The method of any one of claims 38-40, wherein the percentage of fetal hemoglobin in a blood sample obtained from the subject is increased following treatment of the subject with the vector.

51. A method for increasing the amount or percentage of fetal hemoglobin in a subject, the method comprising administering a vector of any one of claims 1-25 or an RNA transcript of any one of claims 26-41 to a cell, thereby increasing the amount or percentage of fetal hemoglobin in the cell.

52. The method of claim 42, wherein the amount or percentage of fetal hemoglobin in a subject is increased by at least 5%.

53. A cell comprising a nucleic acid vector of any one of claims 1-25.

54. A cell expressing or comprising an RNA transcript of any one of claims 26-41.


SEQUENCE LISTING (34 sequences)

SEQ ID NO: 1 - DNA, 52 bp, synthetic construct
  source 1..52: mol_type = other DNA
gatctcacttccccacagaagctcttggcctggcctcctgcagtgccacgct

SEQ ID NO: 2 - DNA, 15 bp, synthetic construct
  source 1..15: mol_type = other DNA
ctccatgtggtagag

SEQ ID NO: 3 - DNA, 84 bp, synthetic construct
  source 1..84: mol_type = other DNA
agtgcggcacatgcttaccagctctaggccagggcagatgggatatgacgaatggactgccagctggatacaaggatgctcacc

SEQ ID NO: 4 - DNA, 21 bp, synthetic construct
  source 1..21: mol_type = other DNA
gcgcgatcgagtgttgaataa

SEQ ID NO: 5 - DNA, 21 bp, synthetic construct
  source 1..21: mol_type = other DNA
ttattcaacactcgatcgcgc

SEQ ID NO: 6 - DNA, 193 bp, synthetic construct
  source 1..193: mol_type = other DNA
gatctcacttccccacagaagctcttggcctggcctcctgcagtgccacgctgcgcgatcgagtgttgaataactccatgtggtagagttattcaacactcgatcgcgcagtgcggcacatgcttaccagctctaggccagggcagatgggatatgacgaatggactgccagctggatacaaggatgctcacc

SEQ ID NO: 7 - DNA, 46 bp, synthetic construct
  source 1..46: mol_type = other DNA
cgcttttcaagccatgcttcctgtgcccccagtggggccctggctg

SEQ ID NO: 8 - DNA, 17 bp, synthetic construct
  source 1..17: mol_type = other DNA
agtttgcgatgagacac

SEQ ID NO: 9 - DNA, 49 bp, synthetic construct
  source 1..49: mol_type = other DNA
agtccgggcacccccagctctggagcctgacaaggaggacaggagagat

SEQ ID NO: 10 - DNA, 21 bp, synthetic construct
  source 1..21: mol_type = other DNA
gctgagcacttagtgtttgta

SEQ ID NO: 11 - DNA, 21 bp, synthetic construct
  source 1..21: mol_type = other DNA
tacaaacactaagtgctcagc

SEQ ID NO: 12 - DNA, 154 bp, synthetic construct
  source 1..154: mol_type = other DNA
cgcttttcaagccatgcttcctgtgcccccagtggggccctggctggctgagcacttagtgtttgtaagtttgcgatgagacactacaaacactaagtgctcagcagtccgggcacccccagctctggagcctgacaaggaggacaggagagat

SEQ ID NO: 13 - DNA, 21 bp, synthetic construct
  source 1..21: mol_type = other DNA
acgggtacttttcattcgcgc

SEQ ID NO: 14 - DNA, 21 bp, synthetic construct
  source 1..21: mol_type = other DNA
gcgcgaatgaaaagtacccgt

SEQ ID NO: 15 - DNA, 154 bp, synthetic construct
  source 1..154: mol_type = other DNA
cgcttttcaagccatgcttcctgtgcccccagtggggccctggctgacgggtacttttcattcgcgcagtttgcgatgagacacgcgcgaatgaaaagtacccgtagtccgggcacccccagctctggagcctgacaaggaggacaggagagat

SEQ ID NO: 16 - DNA, 154 bp, synthetic construct
  source 1..154: mol_type = other DNA
cgcttttcaagccatgcttcctgtgcccccagtggggccctggctggcgcgatcgagtgttgaataaagtttgcgatgagacacttattcaacactcgatcgcgcagtccgggcacccccagctctggagcctgacaaggaggacaggagagat

SEQ ID NO: 17 - DNA, 64 bp, synthetic construct
  source 1..64: mol_type = other DNA
acgctcgcacagaacactcatggattctccatgtggtagagaatccatgagtgttctgtgcgag

SEQ ID NO: 18 - DNA, 64 bp, synthetic construct
  source 1..64: mol_type = other DNA
cgcactcgcacagaacactcatggattctctaccacatggagaatccatgagtgttctgtgcga

SEQ ID NO: 19 - DNA, 64 bp, synthetic construct
  source 1..64: mol_type = other DNA
acgctccagaggatgacgattgtttactccatgtggtagagtaaacaatcgtcatcctctggag

SEQ ID NO: 20 - DNA, 64 bp, synthetic construct
  source 1..64: mol_type = other DNA
cgcactccagaggatgacgattgtttactctaccacatggagtaaacaatcgtcatcctctgga

SEQ ID NO: 21 - DNA, 64 bp, synthetic construct; identical to SEQ ID NO: 17
  source 1..64: mol_type = other DNA

SEQ ID NO: 22 - DNA, 64 bp, synthetic construct; identical to SEQ ID NO: 18
  source 1..64: mol_type = other DNA

SEQ ID NO: 23 - DNA, 6537 bp, synthetic construct
  source 1..6537: mol_type = other DNA
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatggccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcagtccattcgtcatatcccatctgccctggcctagagctggtaagcatgtgccgcactcaacaagatgaagagcaccaactctaccacatggagttggtgctcttcatcttgttgagcgtggcactgcaggaggccaggccaagagcttctgtggggaagtgagatcccccgggggaattcgatatcaagcttatcgataccgtcgacctcgagggggggccgcttacttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgccgccgtcctcgatgttgtggcggatcttgaagttggccttgatgccgttcttctgcttgtcggcggtgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagcccagggtggtcacgagggtgggccagggcacgggcagcttgccggtggtgcagatcagcttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgccatccacgtgatcgatgctagctatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccggggggttcaagcaattctcctgcctcagccaccctaatagctgggattacaggtgcacaccaccatgccaggctaatttttgtactttttgtagaggttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccgtactcgaggtttaaacagtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 24 - DNA, 6682 bp, synthetic construct
  source 1..6682: mol_type = other DNA
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatggccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggcgctctagaactagtggatcccccgggggaattcgcccttacgcgtcgacgtcggtgagcatccttgtatccagctggcagtccattcgtcatatcccatctgccctggcctagagctggtaagcatgtgccgcactgcgcgatcgagtgttgaataactctaccacatggagttattcaacactcgatcgcgcagcgtggcactgcaggaggccaggccaagagcttctgtggggaagtgagatcccccgggggaattcgatatcaagcttatcgataccgtcgacctcgagggggggccgcttacttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgccgccgtcctcgatgttgtggcggatcttgaagttggccttgatgccgttcttctgcttgtcggcggtgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagcccagggtggtcacgagggtgggccagggcacgggcagcttgccggtggtgcagatcagcttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgccatccacgtgatcgatgctagctatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccggggggttcaagcaattctcctgcctcagccaccctaatagctgggattacaggtgcacaccaccatgccaggctaatttttgtactttttgtagaggttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccgtactcgaggtttaaacagtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 25 - DNA, 6582 bp, synthetic construct
  source 1..6582: mol_type = other DNA
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatggccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggcgctctagaactagtggatcccccgggggaattcgcccttacgcgtatctctcctgtcctccttgtcaggctccagagctgggggtgcccggactgctgagcacttagtgtttgtagtgtctcatcgcaaacttacaaacactaagtgctcagccagccagggccccactgggggcacaggaagcatggcttgaaaagcgacctcgagggggggccgcttacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatgccatccacgtgatcgatgctagctatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccggggggttcaagcaattctcctgcctcagccaccctaatagctgggattacaggtgcacaccaccatgccaggctaatttttgtactttttgtagaggttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccgtactcgaggtttaaacagtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 26 - DNA, 6582 bp, synthetic construct
  source 1..6582: mol_type = other DNA
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatggccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggcgctctagaactagtggatcccccgggggaattcgcccttacgcgtatctctcctgtcctccttgtcaggctccagagctgggggtgcccggactacgggtacttttcattcgcgcgtgtctcatcgcaaactgcgcgaatgaaaagtacccgtcagccagggccccactgggggcacaggaagcatggcttgaaaagcgacctcgagggggggccgcttacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatgccatccacgtgatcgatgctagctatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccggggggttcaagcaattctcctgcctcagccaccctaatagctgggattacaggtgcacaccaccatgccaggctaatttttgtactttttgtagaggttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccgtactcgaggtttaaacagtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 27 - DNA, 6851 bp, synthetic construct
  source 1..6851: mol_type = other DNA
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatggccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggcgctctagaactagtggatcccccgggggaattcgcccttacgcgtatctctcctgtcctccttgtcaggctccagagctgggggtgcccggactgctgagcacttagtgtttgtagtgtctcatcgcaaacttacaaacactaagtgctcagccagccagggccccactgggggcacaggaagcatggcttgaaaagcgacctcgaggacgcgtcgacgtcggtgagcatccttgtatccagctggcagtccattcgtcatatcccatctgccctggcctagagctggtaagcatgtgccgcactgcgcgatcgagtgttgaataactctaccacatggagttattcaacactcgatcgcgcagcgtggcactgcaggaggccaggccaagagcttctgtggggaagtgagatcccccgggggaattcgatatcaagcttatcgataccgtcgacctcgagggggggccgcttacttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgccgccgtcctcgatgttgtggcggatcttgaagttggccttgatgccgttcttctgcttgtcggcggtgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagcccagggtggtcacgagggtgggccagggcacgggcagcttgccggtggtgcagatcagcttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgccatccacgtgatcgatgctagctatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccggggggttcaagcaattctcctgcctcagccaccctaatagctgggattacaggtgcacaccaccatgccaggctaatttttgtactttttgtagaggttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccgtactcgaggtttaaacagtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 28 - DNA, 6851 bp, synthetic construct
  source 1..6851: mol_type = other DNA
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatggccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggcgctctagaactagtggatcccccgggggaattcgcccttacgcgtatctctcctgtcctccttgtcaggctccagagctgggggtgcccggactacgggtacttttcattcgcgcgtgtctcatcgcaaactgcgcgaatgaaaagtacccgtcagccagggccccactgggggcacaggaagcatggcttgaaaagcgacctcgaggacgcgtcgacgtcggtgagcatccttgtatccagctggcagtccattcgtcatatcccatctgccctggcctagagctggtaagcatgtgccgcactgcgcgatcgagtgttgaataactctaccacatggagttattcaacactcgatcgcgcagcgtggcactgcaggaggccaggccaagagcttctgtggggaagtgagatcccccgggggaattcgatatcaagcttatcgataccgtcgacctcgagggggggccgcttacttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgccgccgtcctcgatgttgtggcggatcttgaagttggccttgatgccgttcttctgcttgtcggcggtgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagcccagggtggtcacgagggtgggccagggcacgggcagcttgccggtggtgcagatcagcttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgccatccacgtgatcgatgctagctatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccggggggttcaagcaattctcctgcctcagccaccctaatagctgggattacaggtgcacaccaccatgccaggctaatttttgtactttttgtagaggttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccgtactcgaggtttaaacagtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 29 - DNA, 20 bp, synthetic construct
  source 1..20: mol_type = other DNA
caggactcggcttgctgaag

SEQ ID NO: 30 - DNA, 20 bp, synthetic construct
  source 1..20: mol_type = other DNA
tcccccgcttaatactgacg

SEQ ID NO: 31 - DNA, 19 bp, synthetic construct
  source 1..19: mol_type = other DNA
  modified_base 1: mod_base = OTHER; note = fluorescein cytidine
cgcacggcaagaggcgagg

SEQ ID NO: 32 - DNA, 24 bp, synthetic construct
  source 1..24: mol_type = other DNA
gaagttcaggttaattagctgctg

SEQ ID NO: 33 - DNA, 23 bp, synthetic construct
  source 1..23: mol_type = other DNA
tggcaccttaacatttggttctg

SEQ ID NO: 34 - DNA, 24 bp, synthetic construct
  source 1..24: mol_type = other DNA
  modified_base 1: mod_base = OTHER; note = 2'-chloro-7'-phenyl-1,4-dichloro-6-carboxyfluoresce in adenosine
acgaacttcttggagttgtttgct